(19) United States
(12) Patent Application Publication
(10) Pub. No.: US 2025/0281421 A1
(43) Pub. Date: Sep. 11, 2025

(54) COMPOSITIONS FOR SUPPLEMENTING PRODUCTS WITH THERAPEUTIC AGENTS AND METHODS OF USE THEREOF

(71) Applicant: DISRUPTION LABS, INC., Austin, TX (US)

(72) Inventor: Adam GARLAND, Austin, TX (US)

(73) Assignee: DISRUPTION LABS, INC., Austin, TX (US)

(21) Appl. No.: 18/950,994

(22) Filed: Nov. 18, 2024

Related U.S. Application Data
(60) Provisional application No. 63/599,694, filed on Nov. 16, 2023.
(60) Provisional application No. 63/599,726, filed on Nov. 16, 2023.

(51) Int. Cl. A61K 9/51; A61K 9/1271; A61K 9/1277; A61K 31/01; A61K 31/05; A61K 31/12; A61K 31/202; A61K 31/593; A61K 36/185; C12C 5/02

(52) U.S. Cl. CPC A61K 9/5123; A61K 9/1271; A61K 9/1277; A61K 9/5115; A61K 9/5161; A61K 9/5176; A61K 31/01; A61K 31/05; A61K 31/12; A61K 31/202; A61K 31/593; A61K 36/3486; C12C 5/02

(57) ABSTRACT

Several embodiments pertain to nanoparticle-based compositions and their use in methods for the delivery of active agents to subjects and to products. In several embodiments, the compositions are stable for prolonged periods of time and provide enhanced bioavailability and/or dispersibility.

This application claims the benefit of priority from U.S. Provisional Application No. 63/599,694, filed on Nov. 16, 2023, and U.S. Provisional Application No. 63/599,726, filed on Nov. 16, 2023, the entire contents of which are hereby incorporated by reference.

### BACKGROUND

### I. Field of the Disclosure

This disclosure relates to the fields of nanoparticles, drug delivery, and medicine.

### II. Background

Currently, a number of hydrophobic compounds are used as pharmaceuticals, nutraceuticals, cosmetics, dietary supplements, food additives, fragrances, etc. Pure isolate forms of plant extracts may be highly hydrophobic and have different characteristics than their impure counterparts from a formulation and pharmacokinetic standpoint. Use of hydrophobic compounds to treat, prevent, or cure disease and conditions has drawbacks such as poorer bioavailability due to low solubility in water.

Attempts have been made to produce delivery systems for hydrophobic compounds. However, delivery systems for active ingredients can also suffer from a variety of problems. For example, current nanoparticle technology can often be too unstable for effective use. Current nanoparticle formulations may distribute unevenly through a solution or a liquid medium, which may cause uneven delivery of the nanoparticle. The uneven distribution may cause the nanoparticles to form a layer or precipitate out of a solution. Current nanoparticles may also separate over time. The distribution of nanoparticles and/or the active ingredients within current nanoparticle formulation may also suffer from instability. In addition, some nanoparticles rely on the use of synthetic compounds for stability, which can make the nanoparticles unsuitable or undesirable for topical and/or internal use on humans.

Several embodiments disclosed herein solve these or other problems by providing novel and innovative nanoparticle compositions and methods for generating such compositions.

### BRIEF SUMMARY

Applicant has found that compositions as described herein can provide stable nanoparticles capable of carrying a broad range of compounds of interest, such as active agents, hydrophobic compounds, hydrophilic compounds, plant extracts, etc. In some instances the nanoparticles are made of only natural materials (e.g., do not contain compounds that are not produced in nature). In some instances, the nanoparticles may stay stable over months of storage, may stay in suspension over months of storage, and/or may reduce degradation of the compounds of interest as compared to the compounds of interest not in a nanoparticle or in a different nanoparticle. In some instances, the nanoparticles may be in two or more different forms of nanoparticles (e.g., liposome, micelle, nanoemulsion, multi-lamellar, double liposome, solid lipid particles). In some instances, the nanoparticles are simpler to produce, such as requiring no or less microfluidization or high-pressure homogenization. In some instances, the concentration of the compound on interest is may be higher than that which is achievable with other nanoparticle formulations or in an aqueous solution without a nanoparticle.

Certain aspects of the disclosure comprise a nanoparticle composition, including water at about 40 wt. % to about 93.998 wt. % and at least one nanoparticle, the nanoparticle comprising: at least one active agent at 0.001 wt. % to 20 wt. %; at least one phospholipid at 1 wt. % to 25 wt. %; at least one oil other than the at least one phospholipid at 3 wt. % to 50 wt. %; and at least one saponin at 0.001 wt. % to 5 wt. %, wherein the weight percentages are based on the weight of the nanoparticle composition. In some aspects, the ratio of the phospholipid to the at least one oil other than the at least one phospholipid is 1:0.5 to 1:10.

In some instances, the active agent is a pharmaceutical, nutraceutical, cosmetic, pigment, flavoring, a hydrophobic active ingredient, a small molecule, an extract, and/or a biologic. In some instances, the nanoparticle composition does not comprise a starch, a self-emulsifying oil, a co-solvent, a polyethylene glycol, a polymer, glycerin, petroleum-based polymer, a synthetic surfactant, and/or a sterol other than those present in a plant extract, the source of the saponin, and/or from the source of the phospholipid. In some instances, the phospholipid is a lysophospholipid, a phosphatidylcholine, a phosphatidylserine, a phosphatidylinositol, a phosphatidylethanolamine, a phosphatidic acid, a hydrogenated and/or non-hydrogenated soybean phosphatidylcholine, a sunflower phosphatidylcholine, and/or is from a lecithin. In some instances, the oil other than the at least one phospholipid is comprised in, or comprises, a plant and/or vegetable oil, olive oil, sunflower oil, avocado oil, and/or Vitamin E.

In some instances, the saponin is comprised in a plant extract that comprises a water, alcohol, CO₂, or organic solvent extracted plant extract. In some instances, the saponin is comprised in a plant extract that is enriched in glycosides by the extraction. In some instances, the saponin is comprised in a plant extract that is enriched in terpenoid glycosides. In some instances, the saponin is comprised in a plant extract that is a de-oiled plant extract (e.g., an extract that has had oil at least partially removed, such as 50% of the oil removed, 60% removed, 70% removed, 80% removed, 90% removed, 95% removed, 98% removed, 99% removed, 100% removed, etc.). In some instances, the saponin is comprised in a plant extract that is derived from Tribulus terrestris, Yucca schidigera, Quillaja saponaria, Camillia sinesis, and/or Glycyrrhiza glabra.

In some instances, the nanoparticle composition includes a mixture of nanoparticles selected from at least two of a multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, micelles, and/or combinations thereof. In some instances, the nanoparticle composition is in a liquid formulation. In some instances, the nanoparticle composition also includes a co-emulsifier and/or a preservative. In some instances, the nanoparticle includes sodium benzoate, potassium sorbate, sodium ascorbate, an active agent, Vitamin E, olive oil, saponin, phosphatidylcholine, and/or water. In some instances, the active agent is a hydrophobic agent. In some instances, the saponin is comprised in a plant extract that is a de-oiled plant extract. In some instances, the saponin is comprised in a plant extract that is derived from Camellia sinesis and/or Tribulus terrestris. In some instances, the saponin is comprised in a plant extract that is a de-oiled extract of Camellia sinesis and/or Tribulus terrestris.

Certain aspects of the disclosure comprise a nanoparticle composition comprising water at about 40 wt. % to about wt. 93.998% and at least one nanoparticle, the nanoparticle comprising: at least one phospholipid at 1 wt. % to 25 wt. %; at least one oil other than the at least one phospholipid at 3 wt. % to 50 wt. %; and at least one saponin at 0.001 wt. % to 5 wt. %, wherein the weight percentages are based on the weight of the composition. In some aspects, the ratio of the phospholipid to the at least one oil other than the at least one phospholipid is 1:0.5 to 1:10.

Certain aspects of the disclosure comprise a nanoparticle composition comprising water at about 0 wt. % to about wt. 10% and at least one nanoparticle, the nanoparticle comprising: optionally at least one active agent at 0.0015 wt. % to 40 wt. %; at least one phospholipid at 1.5 wt. % to 30 wt. %; at least one oil other than the at least one phospholipid at 7.5 wt. % to 80 wt. %; and at least one saponin at 0.0015 wt. % to 10 wt. %. In some aspects, the ratio of the phospholipid to the at least one oil other than the at least one phospholipid is 1:0.5 to 1:10.

Certain aspects of the disclosure comprise a method of manufacturing a nanoparticle composition, the method comprising the steps of: (a) adding together one or more phospholipid, one or more oil other than the at least one phospholipid, one or more saponin, optionally one or more active agents, and optionally water; (b) mixing the ingredients of step (a) creating a mixture; (c) homogenizing the mixture creating a homogenized mixture; (d) optionally performing microfluidization on the homogenized mixture creating a microfluid; (e) optionally sonicating the microfluid creating a sonicated microfluid; (f) optionally stirring the sonicated microfluid creating a stirred microfluid; (g) optionally creating a coacervation from the stirred microfluid; and (h) optionally precipitating the coacervation.

In some instances, the mixing of step (b) comprises high sheer mixing. In some instances, the homogenizing of step (c) comprises high pressure homogenization. In some instances, the stirring of step (f) comprises mechanical stirring. In some instances, the precipitating of step (h) comprises solvent precipitation. In some instances, the method further comprises extruding the composition using hot melt extrusion. In some instances, the method further involves drying the nanoparticle composition. In some instances, drying comprises lyophilizing, spray drying, fluid bed drying, and/or desiccating the nanoparticle composition.

Certain aspects of the disclosure comprise a method of treating a disease, a disorder, and/or a symptom in an individual, the method comprising administering to the individual a therapeutically effective amount of the nanoparticle composition.

Certain aspects of the disclosure comprise a method of distributing an active agent in a solution, the method comprising contacting the solution with the nanoparticle composition comprising an active agent. Certain aspects of the disclosure comprise a method of adjusting a density of a nanoparticle composition, the method comprising adjusting the density of the nanoparticle composition by adjusting a ratio of at least two of a liposome, a micelle, a nanoemulsion, a multi-lamellar particle, a double liposome particle, and a solid lipid particle.

Applicant has found that compositions as described herein can provide stable nanoparticles capable of carrying a broad range of compounds of interest, such as active agents, hydrophobic compounds, hydrophilic compounds, plant extracts, etc. In some instances the nanoparticles are made of only natural materials (e.g., do not contain compounds that are not found in nature). In some instances, the nanoparticles may stay stable over months of storage, may stay in suspension over months of storage, and/or may reduce degradation of the compounds of interest as compared to the compounds of interest not in a nanoparticle or in a different nanoparticle. In some instances, the nanoparticles may be in two or more different forms of nanoparticles (e.g., liposome, micelle, nanoemulsion, multi-lamellar, double liposome, solid lipid particles). In some instances, the nanoparticles are simpler to produce, such as requiring no or less microfluidization or high-pressure homogenization. In some instances, the concentration of the compound of interest may be higher than that which is achievable with other nanoparticle formulations or in an aqueous solution without a nanoparticle.

Certain aspects of the disclosure comprise a composition comprising a plurality of lipid nanoparticles comprising: 5 to 60 wt. % of at least one phosphatidylcholine; 0 to 40 wt. % of a bulking agent; 0.01 to 20 wt. % of an active agent; and 0.1 to 20 wt. % of at least one saponin. In some instances, the composition comprises 5 to 30 wt. % of a phosphatidylcholine; 0 to 20 wt. % of a bulking agent; 0.1 to 10 wt. % of an active agent; 0.5 to 10 wt. % of at least one saponin; and 25.0 to 93.9 wt. % of water. In some instances, the composition does or does not include a bulking agent. In some instances, the composition comprises about 3.0 wt. % to about 9.0 wt. % of the bulking agent. In some instances, the bulking agent comprises a carbohydrate, polymer, maltodextrin, and/or mannitol. In some instances, the at least one saponin is comprised in a plant extract derived from Tribulus terrestris, Yucca schidigera, Quillaja saponaria, Camellia sinesis, and/or Glycyrrhiza glabra. In specific instances, the at least one saponin is comprised in a plant extract that is a de-oiled plant extract.

In some instances, the composition further comprises 1-Lysophosphatidylcholine (1-LPC), 2-Lysophosphatidylcholine (2-LPC), Phosphatidylethanolamine (PE), N-acylphosphatidylethanolamine (APE), Phosphatidylinositol (PI), and/or Phosphatidic acid (PA). In some instances, the concentration of PI and/or PE is significantly greater than the concentration of 1-LPC, 2-LPC, APE, and/or PA. In some instances, the concentration of 1-LPC, 2-LPC, PE, APE, PI, and/or PA are at or below 1/10^{th} the concentration of phosphatidylcholine. In some instances, the composition comprises about 0 wt. % to about 5.0 wt. % and/or about 0 wt. % to about 0.5 wt. % of an antioxidant. In some instances, greater than about 80% of the active agent is comprised in a lipid nanoparticle.

In some instances, the composition further comprises at least one or more buffers, one or more solvents, and/or one or more preservatives. In specific instances, the one or more buffers comprises sodium bicarbonate and/or sodium carbonate, the one or more solvents comprises ethanol, and the one or more preservatives comprises citric acid monohydrate, potassium sorbate, sodium benzoate, and/or a natural preservative. In some instances, the composition comprises sodium bicarbonate at about 0.0015 wt % to about 0.06 wt. % and/or sodium carbonate at about 0.0015 wt % to about 0.06 wt. %; and citric acid monohydrate at about 0.003 wt. % to about 0.4 wt. %, potassium sorbate at about 0.003 wt. % to about 0.4 wt. %, sodium benzoate at about 0.003 wt. to about 0.4 wt %, and/or natural preservative at about 0.003 wt. % to about 2.0 wt. %. In some instances, the natural preservative comprises a mushroom extract. In some instances, the mushroom extract is from a stem of a white button mushroom.

In some instances, the active agent comprises a hydrophilic active agent or a hydrophobic active agent. In some instances, the active agent has a greater wt. % solubility in either water or ethanol than in medium chain triglycerides at room temperature, neutral pH, and atmospheric pressure. In some instances, the active agent comprises a polyphenol and/or a flavonoid. In specific instances, the flavonoid is a prenylated flavonoid. In specific instances, the prenylated flavonoid is xanthohumol. In some instances, the active agent comprises xanthohumol and additional hop prenylated flavonoids. In some instances, at least about 90% of the xanthohumol does not isomerize to isoxanthohumol when stored at 25° C. with a relative humidity of about 60%, for at least 1 month. In some instances, at least about 90% of the xanthohumol does not isomerize to isoxanthohumol when stored at 25° C. with a relative humidity of 60%, for at least 4 months. In some instances, at least about 90% of the xanthohumol does not isomerize to isoxanthohumol when stored at 25° C. with a relative humidity of 60%, for at least 8 months.

In some instances, the weight ratio of the phosphatidylcholine to the active agent is about 12:1 to about 3:2, or about 11:1 to about 5:1. In some instances, the phosphatidylcholine is from a sunflower. In some instances, the phosphatidylcholine comprises a compound with a structure of:

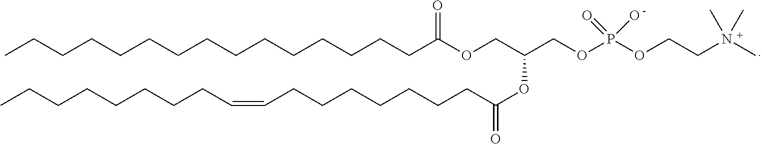

In some instances, the composition comprises: 5 to 25 wt. % of the phosphatidylcholine; 0.1 to 10 wt. % of the active agent; 0 to 7.5 wt. % of the bulking agent; 2.5 to wt. 10% of at least one saponin; and 25 to 89.9 wt. % water. In some instances, the composition comprises: 10 to 25 wt. % the phosphatidylcholine; 1 to about 2.5 wt. % of the active agent; 0 to 5 wt. % the bulking agent; 5 to wt. 10% of at least one saponin; and 50 to about 70 wt. % water.

In some instances, the composition consists essentially of the phosphatidylcholine, the active agent, the bulking agent, the at least one saponin, and optionally the water. In some instances, the composition consists of the phosphatidylcholine, the active agent, the bulking agent, the at least one saponin, and the water. In some instances, the composition further comprises about 0 wt. % to about 0.5 wt. % of an antioxidant. In some instances, the composition comprises 0.01 to 5.0 wt. % or 0.1 to 1.0 wt. % of ethanol. In some instances, the composition further comprises the bulking agent comprises a maltodextrin based bulking agent. In some instances, the composition further comprises the bulking agent comprises a mannitol based bulking agent. In some instances, the plurality of lipid nanoparticles has an average density of about 0.993 g/cm³ to about 1.02 g/cm³. In some instances, the lipid nanoparticles comprise liposomes and/or emulsion particles. In some instances, at least about 90% of the plurality of lipid nanoparticles are liposomes. In some instances, the plurality of lipid nanoparticles has an average size ranging from about 30 nanometers (nm) to about 200 nm, about 50 nm to about 150 nm, and/or about 100 nm to about 150 nm. In some instances, 25% to 100%, 50% to 100%, 75% to 100%, and/or 95% to 100% of the lipid nanoparticles are liposomes.

In some instances, the active agent is comprised within either an inner surface of the nanoparticle, within an outer surface of the nanoparticle, and/or within a lipid bilayer of the nanoparticle. In specific instances, the active agent is comprised within the lipid bilayer of the nanoparticle. In some instances, the majority of nanoparticles are unilamellar. In some instances, the composition comprising multilamellar nanoparticles. In some instances, less than about 20% or less than about 10% of the lipid nanoparticles are emulsion particles.

In some instances, the composition has a turbidity of about 0 to about 5000 Nephelometric Turbidity Units (NTU) at a temperature of about 4° C. and/or a turbidity of about 0 to about 5000 NTU at a temperature of about 40° C. at a relative humidity of about 75%. In some instances, the composition has a turbidity of about 0 to about 2000 NTU at a temperature of about 4° C. and/or a turbidity of about 0 to about 2000 NTU at a temperature of about 40° C. at a relative humidity of about 75%. In some instances, the composition has a turbidity of about 100 to about 1000 NTU at a temperature of about 4° C. and/or a turbidity of about 100 to about 1000 NTU at a temperature of about 40° C. at a relative humidity of about 75%. In some instances, the composition has a turbidity of about 100 to about 500 NTU at a temperature of about 4° C. and/or a turbidity of about 100 to about 800 NTU at a temperature of about 40° C. at a relative humidity of about 75%. In some instances, the composition has a turbidity of about 100 to about 500 NTU at a temperature of about 4° C. and/or a turbidity of about 100 to about 800 NTU at a temperature of about 40° C., when stored for about 1 month at a relative humidity of about 75%.

In some instances, the composition does not include a sterol and/or a triglyceride. In some instances, the composition is free of synthetic surfactants and is free of synthetic emulsifiers at concentrations greater than about 1/10^{th} that of phosphatidylcholine. In some instances, the composition comprises less than about 2.5 wt. % of emulsifiers other than phosphatidylcholine. In some instances, the composition is configured such that, upon storage for a period of one month at room temperature, the average size of the nanoparticles changes by less than about 20%. In some instances, the polydispersity index (PDI) of the plurality of nanoparticles in the composition is about 0.01 to about 0.8, about 0.05 to about 0.5, and/or about 0.1 to about 0.5.

Certain aspects of the disclosure comprise an aqueous formulation comprising the composition described herein. In some instances, the composition is comprised in a second composition, and does not settle or separate from the second composition when stored for at least one month at a temperature of about 4° C. to about 20° C. In some instances, the aqueous formulation comprises 50 wt. % to about 95.0 wt. %, 60.0 wt. % to about 80.0 wt. %, 8.0 wt. % to about 30.0 wt. %, and/or 17.0 wt. % to about 21.0 wt. % of the lipid nanoparticles.

In some instances, the aqueous formulation has a turbidity of about 0 to about 5000 NTU at a temperature of about 4° C. and/or a turbidity of about 0 to about 5000 NTU at a temperature of about 40° C. at a relative humidity of about 75%. In some instances, the aqueous formulation has a turbidity of about 0 to about 2000 NTU at a temperature of about 4° C. and/or a turbidity of about 0 to about 2000 NTU at a temperature of about 40° C. at a relative humidity of about 75%. In some instances, the aqueous formulation has a turbidity of about 100 to about 1000 NTU at a temperature of about 4° C. and/or a turbidity of about 100 to about 1000 NTU at a temperature of about 40° C. at a relative humidity of about 75%. In some instances, the aqueous formulation has a turbidity of about 100 to about 500 NTU at a temperature of about 4° C. and/or a turbidity of about 100 to about 800 NTU at a temperature of about 40° C. at a relative humidity of about 75%. In some instances, the aqueous formulation has a turbidity of about 100 to about 500 NTU at a temperature of about 4° C. and/or a turbidity of about 100 to about 800 NTU at a temperature of about 40° C., when stored for 1 month at a relative humidity of about 75%.

In some instances, the aqueous formulation does not settle or separate when stored for at least one month at a temperature of about 20° C. In some instances, the aqueous formulation is configured such that, upon storage for a period of one month at room temperature, the average size of the plurality of nanoparticles changes by less than about 20%. In some instances, polydispersity index (PDI) of the plurality of nanoparticles in the aqueous formulation is about 0.01 to about 0.8, about 0.05 to about 0.5, about 0.10 to about 0.5. In some instances, the aqueous formulation maintains a turbidity (EBC) of less than about 20 EBC, less than about 15 EBC, less than about 10 EBC, less than about 7 EBC, less than about 4 EBC, less than about 2.5 EBC, and/or 1 EBC more than a control second composition over 5 days at about 40° C. with about 75% relative humidity, where the control second composition is the same as the aqueous formulation except that the control second composition does not comprise the plurality of nanoparticles. In some instances, the aqueous formulation maintains a turbidity (EBC) of less than about 300%, less than about 250%, less than about 200%, less than about 150%, less than about 125%, less than about 115% and/or less than about 110% of the turbidity of a control second composition over 5 days at about 40° C. with about 75% relative humidity, where the control second composition is the same as the aqueous formulation except that the control second composition does not comprise the plurality of nanoparticles. In some instances, the aqueous formulation maintains a turbidity (EBC) that does not significantly differ from the turbidity (EBC) of a control second composition over 5 days at about 40° C. with about 75% relative humidity, where the control second composition is the same as the aqueous formulation except that the control second composition does not comprise the plurality of nanoparticles.

Certain aspects of the disclosure comprise an ingestible composition comprising the composition described herein. In some instances, the ingestible composition is a beverage, comprising at least about 50.0 wt. % water. In specific instances, the ingestible composition is a fermented beverage, a carbonated beverage and/or a beer. In some instances, the composition or aqueous composition reduces the formation of acetyl aldehyde or beta-damascenone in the beer. In some instances, the composition does or does not include alcohol. In some instances, the composition comprises about 0.01 wt. % to about 10.0 wt % alcohol, about 2.0 wt % to about 9.0 wt. % alcohol, and/or about 3.0 wt. % to about 8.0 wt. % alcohol.

In some instances, the composition comprises an aqueous formulation and the plurality of the nanoparticles. In some instances, the ingestible composition comprises about 10 to about 20 milligrams of the plurality of nanoparticles per about 1 gram of the ingestible composition. In some instances, the turbidity of the composition is not perceivable to the unaided eye. In some instances, the turbidity of the ingestible composition is within about 20% of the turbidity of a control ingestible composition that is the same as the ingestible composition except without the plurality of nanoparticles. In some instances, the turbidity of the composition does not increase by more than about 20%, when stored for 5 days at greater than 38° C., or the turbidity of the composition increases less than about 15%, when stored for at least 5 days at greater than about 38° C. In some instances, the pH of the composition is about 2.5 to 9.5, about 5.5 to about 7.5, about 6.0 to about 7.0, and/or about 6.3 to about 6.7.

Certain aspects of the disclosure comprise a method of making the ingestible composition described herein, the method comprising combining the composition described herein with an ingestible item. In some instances, the ingestible item is a beverage. In specific instances, the beverage is a beer. In some instances, the ingestible composition comprises about 10 to about 20 milligrams of the plurality of nanoparticles per about 1 gram of the ingestible item.

Certain aspects of the disclosure comprise a method of ingesting the ingestible composition described herein by a subject, the method comprising ingesting the ingestible composition. In some instances, ingesting the ingestible composition prevents or treats cancer in the subject. In some instances, ingesting the ingestible composition prevents or treats inflammation in the subject. In some instances, ingesting the ingestible composition reduces low-density lipoprotein levels and/or increases high-density lipoprotein levels in the subject. In some instances, ingesting the ingestible composition aids in recovery from a SARS-CoV-2 infection in the subject.

Certain aspects of the disclosure comprise a method of preserving a perishable composition, the method comprising combining the composition described herein to the perishable composition, wherein the composition preserves the perishable composition. In some instances, the perishable composition is an ingestible composition. In some instances, the perishable composition is a topical skin composition.

Certain aspects of the disclosure comprise a topical skin care composition. In some instances, the topical skin care composition is an emulsion. In specific instances, the emulsion is an oil-in-water emulsion. In some instances, the topical skin care composition comprises at least about 50.0 wt. % water. In some instances, the plurality of nanoparticles is comprised within an aqueous phase of the topical skin care composition.

Certain aspects of the disclosure comprise a method of applying the topical skin care composition to skin, the method comprising applying the topical skin care composition to the skin. In some instances, the method further comprises treating a skin condition. In some instances, the skin condition is a fine line, a wrinkle, uneven skin tone, hyperpigmented skin, or inflamed skin. In some instances, the topical skin care composition reduces skin inflammation.

Aspect 1 is directed to a nanoparticle composition comprising water at about 40 wt. % to about wt. 93.998 wt. % and at least one nanoparticle, the nanoparticle comprising:
at least one active agent at 0.001 wt. % to 20 wt. %;
at least one phospholipid at 1 wt. % to 25 wt. %;
at least one oil other than the at least one phospholipid at 3 wt. % to 50 wt. %; and
at least one saponin at 0.001 wt. % to 5 wt. %,
   wherein the weight percentages are based on the weight of the composition.

Aspect 2 is directed to the nanoparticle composition of Aspect 1, wherein the active agent comprises one or more pharmaceutical, nutraceutical, cosmetic, pigment, or flavoring.

Aspect 3 is directed to the nanoparticle composition of any one of Aspects 1 to 2, wherein the active agent comprises a hydrophobic active ingredient.

Aspect 4 is directed to the nanoparticle composition of any one of Aspects 1 to 3, wherein the active agent comprises coenzyme Q10, a vitamin E, a Vitamin A, a beta carotene, squalene, a vitamin K, a docosahexaenoic acid, a curcuminoid, a phytoceramide, vitamin D2, vitamin D3, and/or an ashwagandha extract.

Aspect 5 is directed to the nanoparticle composition of Aspect 4, wherein the vitamin K comprises a powdered vitamin K or a vitamin K oil.

Aspect 6 is directed to the nanoparticle composition of any one of Aspects 1 to 5, wherein the active agent comprises a small molecule and/or a biologic.

Aspect 7 is directed to the nanoparticle composition of any one of Aspects 1 to 6, wherein the nanoparticle composition does not comprise a starch, a self-emulsifying oil, a co-solvent, a polyethylene glycol, and/or a synthetic surfactant.

Aspect 8 is directed to the nanoparticle composition of any one of Aspects 1 to 7, wherein the phospholipid comprises a lysophospholipid, a phosphatidylcholine, a phosphatidylinositol, a phosphatidylethanolamine, and/or a phosphatidylserine.

Aspect 9 is directed to the nanoparticle composition of any one of Aspects 1 to 8, wherein a source of the phospholipid is a lecithin.

Aspect 10 is directed to the nanoparticle composition of any one of Aspects 1 to 9, wherein the phospholipid comprises hydrogenated and/or non-hydrogenated soybean phosphatidylcholine and/or sunflower phosphatidylcholine.

Aspect 11 is directed to the nanoparticle of Aspect 10, wherein less than 10% of the phosphatidylcholine is hydrogenated.

Aspect 12 is directed to the nanoparticle of any one of Aspects 1 to 11, wherein the oil other than the at least one phospholipid comprises a plant and/or vegetable oil.

Aspect 13 is directed to the nanoparticle of Aspect 12, wherein the plant and/or vegetable oil comprises olive oil, sunflower oil, avocado oil, and/or Vitamin E.

Aspect 14 is directed to the nanoparticle composition of any one of Aspects 1 to 13, wherein the at least one saponin is comprised in a plant extract that is derived from Tribulus terrestris, Yucca schidigera, Quillaja saponaria, Camillia sinesis, and/or Glycyrrhiza glabra.

Aspect 15 is directed to the nanoparticle composition of any one of Aspects 1 to 14, wherein the at least one saponin is comprised in a plant extract that is a de-oiled plant extract.

Aspect 16 is directed to the nanoparticle composition of any one of Aspects 1 to 15, wherein the at least one nanoparticle does not comprise a synthetic surfactant.

Aspect 17 is directed to the nanoparticle composition of any one of Aspects 1 to 16, wherein the composition comprises a mixture of nanoparticles selected from at least two of a multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, micelles, and/or combinations thereof.

Aspect 18 is directed to the nanoparticle composition of any one of Aspects 1 to 17, comprised in a liquid formulation.

Aspect 19 is directed to the nanoparticle composition of any one of Aspects 1 to 18, further comprising at least one co-emulsifier and/or at least one preservative.

Aspect 20 is directed to the nanoparticle composition of Aspect 19, wherein the co-emulsifier is a gum.

Aspect 21 is directed to the nanoparticle composition of Aspect 20, wherein the gum is a xanthan gum and/or a biosaccharide gum.

Aspect 22 is directed to the nanoparticle composition of any one of Aspects 20 to 21, wherein the composition comprises 0.01 wt. % to 5 wt. % of the gum, wherein the weight percentages are based on the weight of the composition.

Aspect 23 is directed to the nanoparticle composition of any one of Aspects 1 to 22, wherein the at least one nanoparticle comprises:
sodium benzoate;
potassium sorbate;
sodium ascorbate;
Vitamin D3;
Vitamin E;
olive oil;
an extract of Camellia sinesis and/or Tribulus terrestris that comprises a saponin;
phosphatidylcholine; and
water.

Aspect 24 is directed to a nanoparticle composition comprising water at about 40 wt. % to about 93.998 wt. % and at least one nanoparticle, the nanoparticle comprising:
at least one phospholipid at 1 wt. % to 25 wt. %;
at least one oil other than the at least one phospholipid at 3 wt. % to 45 wt. %; and
at least one saponin at 0.001 wt. % to 5 wt. %,
   wherein the weight percentages are based on the weight of the composition.

Aspect 25 is directed to the nanoparticle composition of Aspect 24, wherein the phospholipid comprises a lysophospholipid, a phosphatidylcholine, a phosphatidylinositol, a phosphatidylethanolamine, and/or a phosphatidylserine.

Aspect 26 is directed to the nanoparticle composition of any one of Aspects 24 to 25, wherein a source of the phospholipid is lecithin.

Aspect 27 is directed to the nanoparticle composition of any one of Aspects 24 to 26, wherein the phospholipid comprises hydrogenated and/or non-hydrogenated soybean phosphatidylcholine and/or sunflower phosphatidylcholine.

Aspect 28 is directed to the nanoparticle of Aspect 27, wherein less than 10% of the phosphatidylcholine is hydrogenated.

Aspect 29 is directed to the nanoparticle of any one of Aspects 24 to 28, wherein the oil other than the at least one phospholipid comprises a plant and/or vegetable oil.

Aspect 30 is directed to the nanoparticle of Aspect 29, wherein the plant and/or vegetable oil comprises olive oil sunflower oil, avocado oil, and/or Vitamin E.

Aspect 31 is directed to the nanoparticle composition of any one of Aspects 24 to 30, wherein the at least one saponin is comprised in a plant extract that is derived from Tribulus terrestris, Yucca schidigera, Quillaja saponaria, Camillia sinesis, and/or Glycyrrhiza glabra.

Aspect 32 is directed to the nanoparticle composition of any one of Aspects 24 to 31, wherein the at least one saponin is comprised in a plant extract that is a de-oiled plant extract.

Aspect 33 is directed to the nanoparticle composition of any one of Aspects 24 to 32, wherein the at least one nanoparticle does not comprise a synthetic surfactant.

Aspect 34 is directed to the nanoparticle composition of any one of Aspects 24 to 33, wherein the composition comprises a mixture of nanoparticles selected from at least two of a multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, micelles, and/or combinations thereof.

Aspect 35 is directed to the nanoparticle composition of any one of Aspects 24 to 34, comprised in a liquid formulation.

Aspect 36 is directed to the nanoparticle composition of any one of Aspects 24 to 35, further comprising at least one co-emulsifier and/or at least one preservative.

Aspect 37 is directed to the nanoparticle composition of Aspect 36, wherein the co-emulsifier is a gum.

Aspect 38 is directed to the nanoparticle composition of any one of Aspects 24 to 37, wherein the nanoparticle composition does not comprise a starch, a self-emulsifying oil, a co-solvent, a polyethylene glycol, and/or a synthetic surfactant.

Aspect 39 is directed to the nanoparticle composition of any one of Aspects 24 to 38, wherein the at least one nanoparticle comprises:
sodium benzoate;
potassium sorbate;
sodium ascorbate;
Vitamin D3;
Vitamin E;
olive oil;
an extract of Camellia sinesis and/or Tribulus terrestris that comprises a saponin;
phosphatidylcholine; and
water.

Aspect 40 is directed to a method of manufacturing a nanoparticle composition of any one of Aspects 1 to 39, the method comprising the steps of:
(a) adding together one or more phospholipid, one or more oil other than the at least one phospholipid, one or more saponin, optionally one or more active agents, and optionally water;
(b) mixing the ingredients of step (a) creating a mixture;
(c) homogenizing the mixture creating a homogenized mixture;
(d) optionally performing microfluidization on the homogenized mixture creating a microfluid;
(e) optionally sonicating the microfluid creating a sonicated microfluid;
(f) optionally stirring the sonicated microfluid creating a stirred microfluid;
(g) optionally creating a coacervation from the stirred microfluid; and
(h) optionally precipitating the coacervation.

Aspect 41 is directed to the method of Aspect 40, wherein the mixing of step (b) comprises high sheer mixing.

Aspect 42 is directed to the method of any one of Aspects 40 to 41, wherein the homogenizing of step (c) comprises high pressure homogenization.

Aspect 43 is directed to the method of any one of Aspects 40 to 42, wherein the stirring of step (f) comprises mechanical stirring.

Aspect 44 is directed to the method of any one of Aspects 40 to 43, wherein the precipitating of step (h) comprises solvent precipitation.

Aspect 45 is directed to the method of any one of Aspects 40 to 44, further comprising drying the nanoparticle composition.

Aspect 46 is directed to the method of Aspect 45, wherein the drying comprises lyophilizing, spray drying, fluid bed drying, and/or desiccating the nanoparticle composition.

Aspect 47 is directed to a nanoparticle composition comprising water at about 0 wt. % to about wt. 10% and at least one nanoparticle, the nanoparticle comprising:
optionally at least one active agent at 0.0015 wt. % to 40 wt. %;
at least one phospholipid at 1.5 wt. % to 30 wt. %;
at least one oil other than the at least one phospholipid at 7.5 wt. % to 80 wt. %; and
at least one saponin at 0.0015 wt. % to 10 wt. %.

Aspect 48 is directed to the nanoparticle composition of Aspect 47, wherein the active agent comprises one or more pharmaceutical, nutraceutical, cosmetic, pigment, or flavoring.

Aspect 49 is directed to the nanoparticle composition of any one of Aspects 47 to 48, wherein the composition comprises a mixture of nanoparticles selected from at least two of a liposome, a micelle, a nanoemulsion, a multi-lamellar particle, a double liposome particle, and a solid lipid particle.

Aspect 50 is directed to the nanoparticle composition of any one of Aspects 47 to 49, wherein the at least one nanoparticle comprises:
sodium benzoate;
potassium sorbate;
sodium ascorbate;
Vitamin D3;
Vitamin E;
olive oil;
an extract of Camellia sinesis and/or Tribulus terrestris that comprises a saponin; and
phosphatidylcholine.

Aspect 51 is directed to the nanoparticle composition of any one of Aspects 47 to 50, wherein the nanoparticle composition does not comprise a starch, a self-emulsifying oil, a co-solvent, a polyethylene glycol, and/or a synthetic surfactant.

Aspect 52 is directed to a method of treating a disease, a disorder, and/or a symptom in an individual, the method comprising administering to the individual a therapeutically effective amount of the nanoparticle composition of any one of Aspects 1 to 39 or 47 to 51.

Aspect 53 is directed to the method of Aspect 52, wherein the disease is an autoimmune disease, a cancer, a degenerative disease, a blood disease, an infection, and/or a deficiency disease.

Aspect 54 is directed to the method of any one of Aspects 52 to 53, wherein the symptom comprises opioid withdrawal, pain, anxiety, depression, insomnia, inflammation, fever, fatigue, muscle aches, or a combination thereof.

Aspect 55 is directed to the method of any one of Aspects 52 to 54, wherein the administering step comprises local administration.

Aspect 56 is directed to the method of any one of Aspects 52 to 55, wherein the administering step comprises systemic administration.

Aspect 57 is directed to the method of any one of Aspects 52 to 56, wherein the administering step comprises oral administration.

Aspect 58 is directed to the method of any one of Aspects 52 to 57, wherein the therapeutically effective amount of the nanoparticle composition comprises 10 mg/kg of the individual to 200 mg/kg of the individual.

Aspect 59 is directed to a method of distributing an active agent in a solution, the method comprising contacting the solution with the nanoparticle composition of any one of Aspects 1 to 39 or 47 to 51.

Aspect 60 is directed to the method of Aspect 59, wherein the nanoparticle composition comprises a mixture of nanoparticles selected from at least two of a liposome, a micelle, a nano emulsion, a multi-lamellar particle, a double liposome particle, and a solid lipid particle.

Aspect 61 is directed to a method of adjusting a density of a nanoparticle composition, the method comprising adjusting the density of the nanoparticle composition of any one of Aspects 1 to 39 or 47 to 51 by adjusting a ratio of at least two of a liposome, a micelle, a nanoemulsion, a multi-lamellar particle, a double liposome particle, and a solid lipid particle.

Aspect 1 is directed to a composition comprising a plurality of lipid nanoparticles comprising: 5 to 60 wt. % of at least one phosphatidylcholine; 0 to 40 wt. % of a bulking agent; 0.01 to 20 wt. % of an active agent; and 0.1 to 20 wt. % of at least one saponin.

Aspect 2 is directed to the composition of Aspect 1, comprising: 5 to 30 wt. % of a phosphatidylcholine; 0 to 20 wt. % of a bulking agent; 0.1 to 10 wt. % of an active agent; 0.5 to 10 wt. % of at least one saponin; and 25.0 to 93.9 wt. % of water.

Aspect 3 is directed to the composition of Aspects 1 or 2, wherein the composition does not include a bulking agent.

Aspect 4 is directed to the composition of Aspects 1 or 2, wherein the composition comprises a bulking agent.

Aspect 5 is directed to the composition of Aspect 4, wherein the composition comprises about 3.0 wt. % to about 9.0 wt. % of the bulking agent.

Aspect 6 is directed to the composition of Aspects 4 or 5, wherein the bulking agent comprises a carbohydrate and/or polymer.

Aspect 7 is directed to the composition of Aspect 6, wherein the bulking agent comprises maltodextrin and/or mannitol.

Aspect 8 is directed to the composition of Aspects 1 to 7, wherein the at least one saponin is comprised in a plant extract derived from Tribulus terrestris, Yucca schidigera, Quillaja saponaria, Camellia sinesis, and/or Glycyrrhiza glabra.

Aspect 9 is directed to the composition of Aspects 1 to 8, wherein the at least one saponin is comprised in a plant extract that is a de-oiled plant extract.

Aspect 10 is directed to the composition of Aspects 1 to 9, further comprising 1-Lysophosphatidylcholine (1-LPC), 2-Lysophosphatidylcholine (2-LPC), Phosphatidylethanolamine (PE), N-acylphosphatidylethanolamine (APE), Phosphatidylinositol (PI), and/or Phosphatidic acid (PA).

Aspect 11 is directed to the composition of Aspect 10, wherein the concentration of PI and/or PE is significantly greater than the concentration of 1-LPC, 2-LPC, APE, and/or PA.

Aspect 12 is directed to the composition of Aspects 10, wherein the concentration of 1-LPC, 2-LPC, PE, APE, PI, and/or PA are at or below 1/10^{th} the concentration of phosphatidylcholine.

Aspect 13 is directed to the composition of Aspects 1 to 12, comprising: about 0 wt. % to about 5.0 wt. % of an antioxidant.

Aspect 14 is directed to the composition of Aspects 1 to 13, comprising: about 0 wt. % to about 0.5 wt. % of an antioxidant.

Aspect 15 is directed to the composition of Aspects 1 to 14, wherein greater than about 80% of the active agent is comprised in a lipid nanoparticle.

Aspect 16 is directed to the composition of Aspects 1 to 15, wherein the composition further comprises at least one or more buffers, one or more solvents, and/or one or more preservatives.

Aspect 17 is directed to the composition of Aspect 16, wherein the one or more buffers comprises sodium bicarbonate and/or sodium carbonate, the one or more solvents comprises ethanol, and the one or more preservatives comprises citric acid monohydrate, potassium sorbate, sodium benzoate, and/or a natural preservative.

Aspect 18 is directed to the composition of Aspect 17, comprising: sodium bicarbonate at about 0.0015 wt % to about 0.06 wt. % and/or sodium carbonate at about 0.0015 wt % to about 0.06 wt. %; and citric acid monohydrate at about 0.003 wt. % to about 0.4 wt. %, potassium sorbate at about 0.003 wt. % to about 0.4 wt. %, sodium benzoate at about 0.003 wt. to about 0.4 wt %, and/or natural preservative at about 0.003 wt. % to about 2.0 wt. %.

Aspect 19 is directed to the composition of Aspects 17 to 18, wherein the natural preservative comprises a mushroom extract.

Aspect 20 is directed to the composition of Aspect 19, wherein the mushroom extract is from a stem of a white button mushroom.

Aspect 21 is directed to the composition of Aspects 1 to 20, wherein the active agent comprises a hydrophilic active agent.

Aspect 22 is directed to the composition of Aspects 1 to 20, wherein the active agent comprises a hydrophobic active agent.

Aspect 23 is directed to the composition of Aspects 1 to 22, wherein the active agent has a greater wt. % solubility in either water or ethanol than in medium chain triglycerides at room temperature, neutral pH, and atmospheric pressure.

Aspect 24 is directed to the composition of Aspects 1 to 23, wherein the active agent comprises a polyphenol.

Aspect 25 is directed to the composition of Aspects 1 to 24, wherein the active agent comprises a flavonoid.

Aspect 26 is directed to the composition of Aspect 25, wherein the flavonoid is a prenylated flavonoid.

Aspect 27 is directed to the composition of Aspect 26, wherein the prenylated flavonoid is xanthohumol.

Aspect 28 is directed to the composition of Aspects 1 to 27, wherein the active agent comprises xanthohumol and additional hop prenylated flavonoids.

Aspect 29 is directed to the composition of Aspects 27 to 28, wherein at least about 90% of the xanthohumol does not isomerize to isoxanthohumol when stored at 25° C. with a relative humidity of about 60%, for at least 1 month.

Aspect 30 is directed to the composition of Aspect 29, wherein at least about 90% of the xanthohumol does not isomerize to isoxanthohumol when stored at 25° C. with a relative humidity of 60%, for at least 4 months.

Aspect 31 is directed to the composition of Aspect 29, wherein at least about 90% of the xanthohumol does not isomerize to isoxanthohumol when stored at 25° C. with a relative humidity of 60%, for at least 8 months.

Aspect 32 is directed to the composition of Aspects 1 to 31, wherein the weight ratio of the phosphatidylcholine to the active agent is about 12:1 to about 3:2.

Aspect 33 is directed to the composition of Aspect 32, wherein the weight ratio of the phosphatidylcholine to the active agent is about 11:1 to about 5:1.

Aspect 34 is directed to the composition of Aspects 1 to 33, wherein the phosphatidylcholine is from a sunflower.

Aspect 35 is directed to the composition of Aspect 34, wherein the phosphatidylcholine comprises a compound with a structure of:

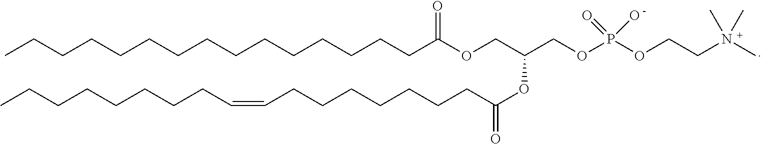

Aspect 36 is directed to the composition of Aspects 1 to 35, comprising: 5 to 25 wt. % of the phosphatidylcholine; 0.1 to 10 wt. % of the active agent; 0 to 7.5 wt. % of the bulking agent; 2.5 to wt. 10% of at least one saponin; and 25 to 89.9 wt. % water.

Aspect 37 is directed to the composition of Aspects 1 to 36, comprising: 10 to 25 wt. % the phosphatidylcholine; 1 to about 2.5 wt. % of the active agent; 0 to 5 wt. % the bulking agent; 5 to wt. 10% of at least one saponin; and 50 to about 70 wt. % water.

Aspect 38 is directed to the composition of Aspects 1 to 37, consisting essentially of the phosphatidylcholine, the active agent, the bulking agent, the at least one saponin, and optionally the water.

Aspect 39 is directed to the composition of Aspects 36 or 37, consisting of the phosphatidylcholine, the active agent, the bulking agent, the at least one saponin, and the water.

Aspect 40 is directed to the composition of Aspects 1 to 39, further comprising: about 0 wt. % to about 0.5 wt. % of an antioxidant.

Aspect 41 is directed to the composition of Aspects 1 to 40, wherein the composition comprises 0.01 to 5.0 wt. % of ethanol.

Aspect 42 is directed to the composition of Aspects 1 to 41, wherein the composition comprises 0.1 to 1.0 wt. % of ethanol.

Aspect 43 is directed to the composition of Aspects 1 to 42, wherein the bulking agent comprises a maltodextrin based bulking agent.

Aspect 44 is directed to the composition of Aspects 1 to 43, wherein the bulking agent comprises a mannitol based bulking agent.

Aspect 45 is directed to the composition of Aspects 1 to 44, wherein the plurality of lipid nanoparticles has an average density of about 0.993 g/cm³ to about 1.02 g/cm³.

Aspect 46 is directed to the composition of Aspects 1 to 45, wherein the lipid nanoparticles comprise liposomes and/or emulsion particles.

Aspect 47 is directed to the composition of Aspect 46, wherein at least about 90% of the plurality of lipid nanoparticles are liposomes.

Aspect 48 is directed to the composition of Aspects 1 to 47, wherein the plurality of lipid nanoparticles has an average size ranging from about 30 nanometers (nm) to about 200 nm.

Aspect 49 is directed to the composition of Aspects 1 to 48, wherein the plurality of lipid nanoparticles has an average size ranging from about 50 nm to about 150 nm.

Aspect 50 is directed to the composition of Aspects 1 to 49, wherein the plurality of lipid nanoparticles has an average size ranging from about 100 nm to about 150 nm.

Aspect 51 is directed to the composition of Aspects 1 to 50, wherein 25% to 100% of the lipid nanoparticles are liposomes.

Aspect 52 is directed to the composition of Aspects 1 to 51, wherein 50% to 100% of the lipid nanoparticles are liposomes.

Aspect 53 is directed to the composition of Aspects 1 to 52, wherein 75% to 100% of the lipid nanoparticles are liposomes.

Aspect 54 is directed to the composition of Aspects 1 to 53, wherein 95% to 100% of the lipid nanoparticles are liposomes.

Aspect 55 is directed to the composition of Aspects 1 to 54, wherein the active agent is comprised within either an inner surface of the nanoparticle, within an outer surface of the nanoparticle, and/or within a lipid bilayer of the nanoparticle.

Aspect 56 is directed to the composition of Aspect 55, wherein the active agent is comprised within the lipid bilayer of the nanoparticle.

Aspect 57 is directed to the composition of Aspects 1 to 56, wherein the majority of nanoparticles are unilamellar.

Aspect 58 is directed to the composition of Aspects 1 to 57, comprising multilamellar nanoparticles.

Aspect 59 is directed to the composition of Aspects 1 to 58, wherein less than about 20% of the lipid nanoparticles are emulsion particles.

Aspect 60 is directed to the composition of Aspects 1 to 59, wherein less than about 10% of the lipid nanoparticles are emulsion particles.

Aspect 61 is directed to the composition of Aspects 1 to 60, having a turbidity of about 0 to about 5000 Nephelometric Turbidity Units (NTU) at a temperature of about 4° C. and/or a turbidity of about 0 to about 5000 NTU at a temperature of about 40° C. at a relative humidity of about 75%.

Aspect 62 is directed to the composition of Aspects 1 to 61, having a turbidity of about 0 to about 2000 NTU at a temperature of about 4° C. and/or a turbidity of about 0 to about 2000 NTU at a temperature of about 40° C. at a relative humidity of about 75%.

Aspect 63 is directed to the composition of Aspects 1 to 62, having a turbidity of about 100 to about 1000 NTU at a temperature of about 4° C. and/or a turbidity of about 100 to about 1000 NTU at a temperature of about 40° C. at a relative humidity of about 75%.

Aspect 64 is directed to the composition of Aspects 1 to 63, having a turbidity of about 100 to about 500 NTU at a temperature of about 4° C. and/or a turbidity of about 100 to about 800 NTU at a temperature of about 40° C. at a relative humidity of about 75%.

Aspect 65 is directed to the composition of Aspects 1 to 64, having a turbidity of about 100 to about 500 NTU at a temperature of about 4° C. and/or a turbidity of about 100 to about 800 NTU at a temperature of about 40° C., when stored for about 1 month at a relative humidity of about 75%.

Aspect 66 is directed to the composition of Aspects 1 to 65, wherein the composition does not include a sterol and/or a triglyceride.

Aspect 67 is directed to the composition of Aspects 1 to 66, wherein the composition is free of synthetic surfactants and is free of synthetic emulsifiers at concentrations greater than about 1/10^{th} that of phosphatidylcholine.

Aspect 68 is directed to the composition of Aspect 67, wherein the composition comprises less than about 2.5 wt. % of emulsifiers other than phosphatidylcholine.

Aspect 69 is directed to the composition of Aspects 1 to 68, wherein the composition is configured such that, upon storage for a period of one month at room temperature, the average size of the nanoparticles changes by less than about 20%.

Aspect 70 is directed to the composition of Aspects 1 to 69, wherein polydispersity index (PDI) of the plurality of nanoparticles in the composition is about 0.01 to about 0.8.

Aspect 71 is directed to the composition of Aspects 1 to 70, wherein PDI of the plurality of nanoparticles in the composition is about 0.05 to about 0.5.

Aspect 72 is directed to the composition of Aspects 1 to 70, wherein PDI of the plurality of nanoparticles in the composition is about 0.1 to about 0.5.

Aspect 73 is directed to an aqueous formulation comprising the composition of any one of claims 1 to 72.

Aspect 74 is directed to the aqueous formulation of Aspect 73, wherein the composition is comprised in a second composition, and does not settle or separate from the second composition when stored for at least one month at a temperature of about 4° C. to about 20° C.

Aspect 75 is directed to the aqueous formulation of Aspects 73 to 74, comprising 20 wt. % to about 99.0 wt. % of the lipid nanoparticles.

Aspect 76 is directed to the aqueous formulation of Aspects 73 to 75, comprising 50 wt. % to about 95.0 wt. % of the lipid nanoparticles.

Aspect 77 is directed to the aqueous formulation of Aspects 73 to 76, comprising 60.0 wt. % to about 80.0 wt. % of the lipid nanoparticles.

Aspect 78 is directed to the aqueous formulation of Aspects 73 to 74, comprising 8.0 wt. % to about 30.0 wt. % of the lipid nanoparticles.

Aspect 79 is directed to the aqueous formulation of Aspect 78, comprising 17.0 wt. % to about 21.0 wt. % of the lipid nanoparticles.

Aspect 80 is directed to the aqueous formulation of Aspects 73 to 79, having a turbidity of about 0 to about 5000 NTU at a temperature of about 4° C. and/or a turbidity of about 0 to about 5000 NTU at a temperature of about 40° C. at a relative humidity of about 75%.

Aspect 81 is directed to the aqueous formulation of Aspects 73 to 80, having a turbidity of about 0 to about 2000 NTU at a temperature of about 4° C. and/or a turbidity of about 0 to about 2000 NTU at a temperature of about 40° C. at a relative humidity of about 75%.

Aspect 82 is directed to the aqueous formulation of Aspects 73 to 81, having a turbidity of about 100 to about 1000 NTU at a temperature of about 4° C. and/or a turbidity of about 100 to about 1000 NTU at a temperature of about 40° C. at a relative humidity of about 75%.

Aspect 83 is directed to the aqueous formulation of Aspects 73 to 82, wherein the aqueous formulation has a turbidity of about 100 to about 500 NTU at a temperature of about 4° C. and/or a turbidity of about 100 to about 800 NTU at a temperature of about 40° C. at a relative humidity of about 75%.

Aspect 84 is directed to the aqueous formulation of Aspects 73 to 83, wherein the aqueous formulation has a turbidity of about 100 to about 500 NTU at a temperature of about 4° C. and/or a turbidity of about 100 to about 800 NTU at a temperature of about 40° C., when stored for 1 month at a relative humidity of about 75%.

Aspect 85 is directed to the aqueous formulation of Aspects 73 to 84, wherein the aqueous formulation does not settle or separate when stored for at least one month at a temperature of about 20° C.

Aspect 86 is directed to the aqueous formulation of Aspects 73 to 85, wherein the aqueous formulation is configured such that, upon storage for a period of one month at room temperature, the average size of the plurality of nanoparticles changes by less than about 20%.

Aspect 87 is directed to the aqueous formulation of Aspects 73 to 86, wherein polydispersity index (PDI) of the plurality of nanoparticles in the aqueous formulation is about 0.01 to about 0.8.

Aspect 88 is directed to the aqueous formulation of Aspects 73 to 87, wherein PDI of the plurality of nanoparticles in the aqueous formulation is about 0.05 to about 0.5.

Aspect 89 is directed to the aqueous formulation of Aspects 73 to 88, wherein PDI of the plurality of nanoparticles in the aqueous formulation is about 0.10 to about 0.5.

Aspect 90 is directed to the aqueous formulation of Aspects 73 to 89, wherein the aqueous formulation maintains a turbidity (EBC) of less than about 20 EBC more than a control second composition over 5 days at about 40° C. with about 75% relative humidity, where the control second composition is the same as the aqueous formulation except that the control second composition does not comprise the plurality of nanoparticles.

Aspect 91 is directed to the aqueous formulation of Aspects 73 to 90, wherein the aqueous formulation maintains a turbidity (EBC) of less than about 15 EBC more than a control second composition over 5 days at about 40° C. with about 75% relative humidity, where the control second composition is the same as the aqueous formulation except that the control second composition does not comprise the plurality of nanoparticles.

Aspect 92 is directed to the aqueous formulation of Aspects 73 to 91, wherein the aqueous formulation maintains a turbidity (EBC) of less than about 10 EBC more than a control second composition over 5 days at about 40° C. with about 75% relative humidity, where the control second composition is the same as the aqueous formulation except that the control second composition does not comprise the plurality of nanoparticles.

Aspect 93 is directed to the aqueous formulation of Aspects 73 to 92, wherein the aqueous formulation maintains a turbidity (EBC) of less than about 7 EBC more than a control second composition over 5 days at about 40° C. with about 75% relative humidity, where the control second composition is the same as the aqueous formulation except that the control second composition does not comprise the plurality of nanoparticles.

Aspect 94 is directed to the aqueous formulation of Aspects 73 to 93, wherein the aqueous formulation maintains a turbidity (EBC) of less than about 4 EBC more than a control second composition over 5 days at about 40° C. with about 75% relative humidity, where the control second composition is the same as the aqueous formulation except that the control second composition does not comprise the plurality of nanoparticles.

Aspect 95 is directed to the formulation of Aspects 73 to 94, wherein the aqueous formulation maintains a turbidity of less than about 2.5 EBC more than a control second composition over 5 days at about 40° C. with about 75% relative humidity, where the control second composition is the same as the aqueous formulation except that the control second composition does not comprise the plurality of nanoparticles.

Aspect 96 is directed to the aqueous formulation of Aspects 73 to 95, wherein the aqueous formulation maintains a turbidity of less than about 1 EBC more than a control second composition over 5 days at about 40° C. with about 75% relative humidity, where the control second composition is the same as the aqueous formulation except that the control second composition does not comprise the plurality of nanoparticles.

Aspect 97 is directed to the aqueous formulation of Aspects 73 to 96, wherein the aqueous formulation maintains a turbidity (EBC) of less than about 300% that of a control second composition over 5 days at about 40° C. with about 75% relative humidity, where the control second composition is the same as the aqueous formulation except that the control second composition does not comprise the plurality of nanoparticles.

Aspect 98 is directed to the aqueous formulation of Aspects 73 to 97, wherein the aqueous formulation maintains a turbidity (EBC) of less than about 250% that of a control second composition over 5 days at about 40° C. with about 75% relative humidity, where the control second composition is the same as the aqueous formulation except that the control second composition does not comprise the plurality of nanoparticles.

Aspect 99 is directed to the aqueous formulation of Aspects 73 to 98, wherein the aqueous formulation maintains a turbidity (EBC) of less than about 200% that of a control second composition over 5 days at about 40° C. with about 75% relative humidity, where the control second composition is the same as the aqueous formulation except that the control second composition does not comprise the plurality of nanoparticles.

Aspect 100 is directed to the aqueous formulation of Aspects 73 to 99, wherein the aqueous formulation maintains a turbidity (EBC) of less than about 150% that of a control second composition over 5 days at about 40° C. with about 75% relative humidity, where the control second composition is the same as the aqueous formulation except that the control second composition does not comprise the plurality of nanoparticles.

Aspect 101 is directed to the aqueous formulation of Aspects 73 to 100, wherein the aqueous formulation maintains a turbidity (EBC) of less than about 125% that of a control second composition over 5 days at about 40° C. with about 75% relative humidity, where the control second composition is the same as the aqueous formulation except that the control second composition does not comprise the plurality of nanoparticles.

Aspect 102 is directed to the aqueous formulation of Aspects 73 to 101, wherein the aqueous formulation maintains a turbidity of less than about 115% that of a control second composition over 5 days at about 40° C. with about 75% relative humidity, where the control second composition is the same as the aqueous formulation except that the control second composition does not comprise the plurality of nanoparticles.

Aspect 103 is directed to the aqueous formulation of Aspects 73 to 102, wherein the aqueous formulation maintains a turbidity of less than about 110% that of a control second composition over 5 days at about 40° C. with about 75% relative humidity, where the control second composition is the same as the aqueous formulation except that the control second composition does not comprise the plurality of nanoparticles.

Aspect 104 is directed to the aqueous formulation of Aspects 73 to 103, wherein the aqueous formulation maintains a turbidity (EBC) that does not significantly differ from the turbidity (EBC) of a control second composition over 5 days at about 40° C. with about 75% relative humidity, wherein the control second composition is the same as the aqueous formulation except that the control second composition does not comprise the plurality of nanoparticles.

Aspect 105 is directed to an ingestible composition comprising the composition of any one of claims 1 to 72.

Aspect 106 is directed to the ingestible composition of Aspect 105, wherein the ingestible composition is a beverage.

Aspect 107 is directed to the ingestible composition of Aspects 105 to 106, comprising at least about 50.0 wt. % water.

Aspect 108 is directed to the ingestible composition of Aspects 105 to 107, wherein the ingestible composition is a fermented beverage.

Aspect 109 is directed to the ingestible composition of Aspects 105 to 108, wherein the ingestible composition is a carbonated beverage.

Aspect 110 is directed to the ingestible composition of Aspects 105 to 109, wherein ingestible composition is a beer.

Aspect 111 is directed to the ingestible composition of Aspect 110, wherein the composition or aqueous composition reduces the formation of acetyl aldehyde or beta-damascenone in the beer.

Aspect 112 is directed to the ingestible composition of Aspects 105 to 111, wherein the composition does not include alcohol.

Aspect 113 is directed to the ingestible composition of Aspects 105 to 112, wherein the composition includes alcohol.

Aspect 114 is directed to the ingestible composition of Aspect 113, wherein the composition comprises about 0.01 wt. % to about 10.0 wt % alcohol.

Aspect 115 is directed to the ingestible composition of Aspect 114, wherein the composition comprises about 2.0 wt. % to about 9.0 wt. % alcohol.

Aspect 116 is directed to the ingestible composition of Aspect 115, wherein the composition comprises about 3.0 wt. % to about 8.0 wt. % alcohol.

Aspect 117 is directed to the ingestible composition of Aspects 105 to 116, wherein the composition comprises an aqueous formulation and the plurality of the nanoparticles.

Aspect 118 is directed to the ingestible composition of Aspects 105 to 117, wherein the ingestible composition comprises about 10 to about 20 milligrams of the plurality of nanoparticles per about 1 gram of the ingestible composition.

Aspect 119 is directed to the ingestible composition of Aspects 105 to 118, wherein the turbidity of the composition is not perceivable to the unaided eye.

Aspect 120 is directed to the ingestible composition of Aspects 105 to 119, wherein the pH of the composition is about 2.5 to 9.5.

Aspect 121 is directed to the ingestible composition of Aspects 105 to 120, wherein the pH of the composition is about 5.5 to about 7.5.

Aspect 122 is directed to the ingestible composition of Aspects 105 to 121, wherein the pH of the composition is about 6.0 to about 7.0.

Aspect 123 is directed to the ingestible composition of Aspects 105 to 122, wherein the pH of the composition is about 6.3 to about 6.7.

Aspect 124 is directed to a method of making the ingestible composition of any one of claims 105 to 123, the method comprising combining the composition of any one of claims 1 to 72 with an ingestible item.

Aspect 125 is directed to the method of Aspect 124, wherein the ingestible item is a beverage.

Aspect 126 is directed to the method of Aspect 125, wherein the beverage is a beer.

Aspect 127 is directed to the method of Aspects 124 to 126, wherein the ingestible composition comprises about 10 to about 20 milligrams of the plurality of nanoparticles per about 1 gram of the ingestible item.

Aspect 128 is directed to a method of ingesting the ingestible composition of any one of claims 105 to 123 by a subject, the method comprising ingesting the ingestible composition.

Aspect 129 is directed to the method of Aspect 128, wherein ingesting the ingestible composition prevents or treats cancer in the subject.

Aspect 130 is directed to the method of Aspects 128 to 129, wherein ingesting the ingestible composition prevents or treats inflammation in the subject.

Aspect 131 is directed to the method of Aspects 128 to 130, wherein ingesting the ingestible composition reduces low-density lipoprotein levels and/or increases high-density lipoprotein levels in the subject.

Aspect 132 is directed to the method of Aspects 128 to 131, wherein ingesting the ingestible composition aids in recovery from a SARS-CoV-2 infection in the subject.

Aspect 133 is directed to a method of preserving a perishable composition, the method comprising combining the composition of any one of claims 1 to 72 to the perishable composition, wherein the composition preserves the perishable composition.

Aspect 134 is directed to the method of Aspect 133, wherein the perishable composition is an ingestible composition.

Aspect 135 is directed to the method of Aspect 133, wherein the perishable composition is a topical skin composition.

Aspect 136 is directed to a topical skin care composition comprising the composition of any one of claims 1 to 72.

Aspect 137 is directed to the topical skin care composition of Aspect 136, wherein the topical skin care composition is an emulsion.

Aspect 138 is directed to the topical skin care composition of Aspect 137, wherein the emulsion is an oil-in-water emulsion.

Aspect 139 is directed to the topical skin care composition of Aspects 136 to 138, wherein the topical skin care composition comprises at least about 50.0 wt. % water.

Aspect 140 is directed to the topical skin care composition of Aspects 136 to 139, wherein the plurality of nanoparticles is comprised within an aqueous phase of the topical skin care composition.

Aspect 141 is directed to a method of applying the topical skin care composition of any one of claims 136 to 140 to skin, the method comprising applying the topical skin care composition to the skin.

Aspect 142 is directed to the method of Aspect 141, further comprising treating a skin condition.

Aspect 143 is directed to the method of Aspect 142, wherein the skin condition is a fine line, a wrinkle, uneven skin tone, hyperpigmented skin, or inflamed skin.

Aspect 144 is directed to the method of Aspects 141 to 143, wherein the topical skin care composition reduces skin inflammation.

It is contemplated that any embodiment discussed in this specification can be implemented with respect to any method or composition of the disclosure, and vice versa. Furthermore, compositions of the disclosure can be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is a flow chart showing an embodiment of a method of preparing a composition as disclosed herein.

FIG. 2 is a flow chart showing another embodiment of a method for preparing a composition as disclosed herein.

### DETAILED DESCRIPTION

Applicant has found that compositions as described herein can provide stable nanoparticles capable of carrying a broad range of compounds of interest, such as active agents, hydrophobic compounds, hydrophilic compounds, plant extracts, etc. In some instances the nanoparticles are made of only natural materials (e.g., do not contain compounds that are not produced in nature). In some instances, the nanoparticles may stay stable over months of storage, may stay in suspension over months of storage, and/or may reduce degradation of the compounds of interest as compared to the compounds of interest not in a nanoparticle or in a different nanoparticle. In some instances, the concentration of the compound of interest may be higher than that which is achievable with other nanoparticle formulations or in an aqueous solution without a nanoparticle. In some instances, the nanoparticles may be in two or more different forms of nanoparticles (e.g., liposome, micelle, nanoemulsion, multi-lamellar, double liposome, solid lipid particles). In some instances, the nanoparticles are simpler to produce, such as requiring no or less microfluidization or high pressure homogenization.

### I. Definitions

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

The terms “approximately,” “about,” and “substantially” as used herein represent an amount close to the stated amount that still performs a desired function or achieves a desired result. For example, in several embodiments, as the context may dictate, the terms “approximately”, “about”, and “substantially” may refer to an amount that is within less than or equal to 10% of the stated amount. The term “generally” as used herein represents a value, amount, or characteristic that predominantly includes or tends toward a particular value, amount, or characteristic.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. When used in the context of a compound, composition or device, the term “comprising” means that the compound, composition or device includes at least the recited features or components but may also include additional features or components. The compositions and methods for their making or use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure. The phrase “consisting of” excludes any element not specified.

The compositions and methods for their making or use can “comprise,” “consist essentially of,” or “consist of” any of the ingredients or steps disclosed throughout the specification.

The terms “treatment,” “treating,” “treat” and the like shall be given its ordinary meaning and shall also include herein to generally refer to obtaining a desired pharmacologic and/or physiologic effect. The effect may be prophylactic in terms of completely or partially preventing a disorder, disease, or symptom thereof and/or may be therapeutic in terms of a partial or complete stabilization or cure for a disorder or disease and/or adverse effect attributable to the disorder or disease. “Treatment” shall also cover any treatment of a disorder or disease in a mammal, particularly a human, and includes: (a) preventing the disorder, disease, or symptom (e.g., of the disorder or disease) from occurring in a subject which may be predisposed to the disorder, disease, or symptom but has not yet been diagnosed as having it; (b) inhibiting the disorder, disease, or symptom, e.g., arresting its development; and/or (c) relieving the disorder, disease, or symptom (e.g., causing regression of the disorder, disease, or symptom).

The “patient” or “subject” treated as disclosed herein may be a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, mini-pigs (a mini-pig is a small breed of swine weighing about 35 kg as an adult), horses, cats, dog, rabbits, rodents (e.g., rats or mice), monkeys, etc. Human subjects include neonates, infants, children, juveniles, adults and geriatric subjects. The subject can be a subject “in need of” the methods disclosed herein can be a subject that is experiencing a disease state and/or is anticipated to experience a disease state, and the methods and compositions of the disclosure are used for therapeutic and/or prophylactic treatment.

As used herein, the terms “active agent”, “active compound”, “pharmaceutical composition”, “therapeutic agent”, and the like may be used interchangeably. The terms generally refer to compositions having pharmacological activity or other direct effects in the diagnosis, cure, mitigation, treatment, or prevention of disease, or to affect the structure, appearance, or any function of molecules, cells, tissues, organs, or subject. The terms may refer to compositions in a beverage. The terms may refer to pharmaceuticals, nutraceuticals, cosmetics, pigments, flavorings, and the like. The terms may refer to compositions that are hydrophobic, hydrophilic, or both.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, ascorbic acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in U.S. Pat. No. 4,783,443 A, Johnston et al., published Sep. 11, 1987 (incorporated by reference herein in its entirety).

An “effective amount” or a “therapeutically effective amount” as used herein can refer to an amount of a therapeutic agent that is effective to relieve, to some extent, or to reduce the likelihood of onset of, one or more of the symptoms of a disease or condition (e.g., disorder), and includes curing a disease or condition. An “effective amount” or a “therapeutically effective amount” refers to that amount of a recited compound and/or composition that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, an effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some embodiments, an improvement in a condition can be a reduction in disease symptoms or manifestations (e.g., pain, anxiety & stress, seizures, malaise, inflammation, mood disorders, insomnia, etc.). Actual dosage levels of active ingredients in an active composition of the presently disclosed subject matter can be varied so as to administer an amount of the active compound(s) that is effective to achieve the desired response for a particular subject and/or application. The selected dosage level will depend upon a variety of factors including, but not limited to, the activity of the composition, composition, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

“Curing” means that the symptoms of a disease or condition are eliminated; however, certain long-term or permanent effects may exist even after a cure is obtained (such as extensive tissue damage).

As used herein, the term “weight percent” (or wt. %, weight %, percent by weight, etc.), when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

As used herein, the “dry weight %” (e.g., “dry wt. %”, “dry weight percent”, etc.) of an ingredient is the weight percent of that ingredient in the composition where the weight of water has not been included in the calculation of the weight percent of that ingredient. A dry weight % can be calculated for and includes either a composition that does not include water (e.g., that has been dried to, for example, a powder) or for a composition that includes water but where the amount of water is not included in the calculation.

As used herein, the “wet weight %” (e.g., “wet wt. %”, “wet weight percent”, etc.) of an ingredient is the weight percent of that ingredient in a composition where the weight of water is included in the calculation of the weight percent of that ingredient. For example, the dry weight percent of component A when 5 grams of component A is added to 95 grams of component B and 100 grams of water is 5% (e.g., 5 g A/(5 g A+95 g B)×100%). Alternatively, the wet weight percent of component A when 5 grams of component A is added to 95 grams of component B and 100 grams of water is 2.5% (e.g., 5 g A/(5 g A+95 g B+100 g water)×100%).

As used herein, the term “weight volume percent” (or weight volume percentage, w/v, w/v (%), etc.), when referring to a component, is the weight of the component in grams divided by the volume of a solution in milliliters that includes the component, multiplied by 100%. For example, the w/v of component A when 5 grams of component A is added to a solution to provide 100 mL of solution is 5 w/v (%) (e.g., 5 g solute A/100 mL solution×100%).

When referring to an amount present for one or more ingredients, the term “collectively or individually” (and variations thereof) means that the amount is intended to signify that the ingredients combined may be provided in the amount disclosed, or each individual ingredient may be provided in the amount disclosed. For example, if agents A and B are referred to as collectively or individually being present in a composition at a wt. % of 5%, that means that A may be at 5 wt. % in the composition, B may be at 5 wt. % in the composition, or the combination of A and B may be present at a total of 5 wt. % (A+B=5 wt. %). Alternatively, where both A and B are present, A may be at 5 wt. % and B may be at 5 wt. %, totaling 10 wt. %.

When referring to the amount present for one or more ingredients, the terms “or ranges including and/or spanning the aforementioned values” (and variations thereof) is meant to include any range that includes or spans the aforementioned values. For example, when the wt. % of an ingredient is expressed as 1%, 5%, 10%, 20%, “or ranges including and/or spanning the aforementioned values,” this includes wt. % ranges for the ingredient spanning from 1% to 20%, 1% to 10%, 1% to 5%, 5% to 20%, 5% to 10%, and 10% to 20%.

As used herein, the term “extract” means a compound or group of compounds that has been extracted from an extract source. For example, an extract source may be a plant (e.g., flavonoids, hops, hemp, cannabis, kratom, kava, Kanna, etc.) or a fungus (e.g., mushrooms, cordyceps, lion mane, reishi, chaga gano, psilocybin mushrooms, etc.). An extract may be extracted from the extract source as a full spectrum extract, a broad spectrum extract, a distillate, or an isolate. Full-spectrum extracts can be made a variety of different ways known in the art, including through pressure along (e.g., using a press, such as a rosin press), solvent extraction (using an appropriate solvent, such as, ethanol, ether, ethyl acetate, acetone, low and medium chain hydrocarbon solvents, etc.), supercritical CO₂ extraction, and the like. Where solvent extraction is used, extract can be collected by removing the extraction solvent medium. Broad spectrum extracts are more refined than full spectrum extracts. Broad spectrum extracts may be made by further purifying full spectrum extracts, removing particular agents from full spectrum extracts, etc. Distillates may be made using methods known in the art, including extracting a full or broad spectrum extract and, optionally performing vacuum filtration to remove insoluble, and preforming a distillation. Alternatively, a distillate may be collected by directly subjecting a source to distillation conditions. An isolate is a single compound that has been isolated in a purified form (including substantially pure forms or pure form).

As used herein, a “therapeutic ingredient” is a compound or group of compounds provided within a composition or as a composition that provides a therapeutic benefit. A therapeutic ingredient in a particular composition may be an extract, a therapeutic agent, or a group of therapeutic agents.

As used herein, a “therapeutic agent” (or “active” or “active agent”) is a compound that provides a therapeutic benefit. One or more therapeutic agents may be combined to provide a therapeutic ingredient in a composition.

As used herein, the “entourage effect” is a mechanism by which the combination of therapeutic agents in extracts or therapeutic ingredients act synergistically to modulate or treat a disease or disorder or exert a therapeutic benefit.

As used herein, the term “phospholipid” refers to a lipid having two hydrophobic fatty acid tails and a hydrophilic head comprising of a phosphate group.

As used herein, the term “short chain triglyceride” refers to tri-substituted triglycerides with fatty acids having aliphatic tails of 1 to 5 carbon atoms (1, 2, 3, 4, 5) and mixtures thereof.

As used herein, the term “medium chain triglyceride” refers to tri-substituted triglycerides with fatty acids having aliphatic tails of 6 to 12 carbon atoms (6, 7, 8, 9, 10, 11, 12) and mixtures thereof.

As used herein, the term “long chain triglyceride” refers to tri-substituted triglycerides with fatty acids having an aliphatic tail of greater than 13 carbon atoms (13, 14, 15, 16, 17, 18, 19, 20, or more) and mixtures thereof.

As used herein, the term “sterol” refers to a subgroup of steroids with a hydroxyl group at the 3-position of the A-ring.

As used herein, the term “Cmax” is given its plain and ordinary meaning and refers to the maximum (or peak) plasma concentration of an agent after it is administered.

As used herein, the term “Tmax” is given its plain and ordinary meaning and refers to the length of time required for an agent to reach maximum plasma concentration after the agent is administered.

As used herein, the term “AUC” is given its plain and ordinary meaning and refers to the calculated area under the curve, referring to a plasma concentration-time curve (e.g., the definite integral in a plot of drug concentration in blood plasma vs. time.).

As used herein, “polydispersity” or “PDI” is used to describe the degree of non-uniformity of a size distribution of particles. Also known as the heterogeneity index, PDI is a number calculated from a two-parameter fit to the correlation data (the cumulants analysis). This index is dimensionless and scaled such that values smaller than 0.05 are mainly seen with highly monodisperse standards.

As used herein, an “amino acid” includes amino acids with natural amino acid side chains or non-natural amino acid side chains. As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine. As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

As used herein, the term “hydrophobic”, “water insoluble”, or “insoluble in water”, or “not soluble in water”, or similar terms can refer to a chemical that has a water solubility below 0.1 g in 100 ml of water at 20° C. and 101.325 kPa of pressure. As used herein, the term “hydrophilic”, or “water soluble”, or “soluble in water”, or similar terms can refer to a chemical that has a water solubility at or above 1 g in 100 ml of water at 20° C. and 101.325 kPa of pressure. As used herein, the term “semi-soluble in water”, “water semi-soluble”, or “semi-insoluble in water”, or “water semi-insoluble”, or similar terms can refer to a chemical that has a water solubility at 0.1 g in 100 ml of water at 20° C. and 101.325 kPa of pressure to below 1 g in 100 ml of water at 20° C. and 101.325 kPa of pressure. As used herein, the term “solubility” refers to the solubility of the ingredient or compound in question in pure solvent at 20° C. and 101.325 kPa of pressure unless otherwise noted.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of any embodiment disclosed herein, but instead as merely intended to highlight alternative or additional features that may or may not be utilized in a particular embodiment of the disclosure. Likewise, a group of items linked with the conjunction “or” should not be read as requiring mutual exclusivity among that group, in some embodiments, but rather should be read as “and/or”.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

Conditional language used herein, such as, among others, “can,” “could,” “might,” “may,” “e.g.,” and the like, unless specifically stated otherwise or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise.

### II. Compositions

Disclosed herein are therapeutic lipid-based particle products comprising therapeutic ingredients. In some instances, a therapeutic ingredient comprises ingredients that have been developed as a pharmaceutical drug. In some instances, a therapeutic ingredient comprises ingredients that have been developed as a natural supplement. In some instances, a therapeutic ingredient comprises vitamins. In some embodiments, a nano-lipid delivery system is utilized to impart apparent aqueous solubility and deliverability to an otherwise practically water insoluble molecule in pure water at room temperature, neutral pH, and atmospheric pressure. In some embodiments, as disclosed herein, attributes of some embodiments disclosed herein have been determined to be high quality and reproducible. Such reproducibility and low variations may allow the products to generate a reproducible certificate of analysis for different batches.

### A. Particles and Lipid Compositions

Certain embodiments disclosed herein concern compositions comprising a nanoparticle, which may encapsulate or encompass a compound of interest, such as an active agent. The compositions, in some embodiments, can deliver highly pure active agents, such as pharmaceuticals, nutraceuticals, cosmetics, pigments, flavorings, etc. in a nanoparticle delivery system (e.g., lipid nanoparticle, a liposomal system, oil-in-water emulsions, dry liposome particles, etc.). Some active agents include hydrophobic active agents. Some nanoparticles do not comprise an active agent. Some active agents include, but are not limited to kratom extracts, kanna extracts, kava extracts, mushroom extracts (e.g., Psilocybe cubensis), Cannabis extracts, cannabinoids, coenzyme Q10, a vitamin E, vitamin A, a beta carotene, squalene, a vitamin K, a docosahexaenoic acid, a curcuminoid, a phytoceramide, vitamin D2, vitamin D3, and/or an ashwagandha extract. Compositions disclosed herein, in some embodiments, comprise mixed nanoparticle compositions comprising active agents or combinations of active agents. In several embodiments, the disclosed compositions, which may be mixed nanoparticle compositions, have certain characteristics including, but not limited to, fewer impurities, fewer variations batch-to-batch (e.g., stability, degradation profiles, efficacy), better delivery predictability, fewer side effects when administered to a subject, higher bioavailability, faster onset of activity, greater long term storage stability of the particles and the active ingredient(s), greater stability at higher temperatures of the particles and the active ingredient(s), better dispersibility, greater stability of a dispersion, better efficacy, higher concentrations of active agents, and/or simpler production methods, relative to the characteristics of compositions known in the art.

Certain embodiments concern nanoparticles, including mixed micelle-based compositions, and their use in methods for the delivery of active compounds, which may include plant extracts and/or other beneficial agents (e.g., vitamins, nutrients, saponins, other plant extracts, nutraceuticals, pharmaceuticals, flavorings, pigments, or other beneficial agents for delivery). In several embodiments, the compositions are stable (e.g., at room temperature) for prolonged periods of time.

Several embodiments disclosed herein pertain to formulations, including mixed nanoparticle compositions, for the delivery of one or more active agents (e.g., active agents) to subjects. Several embodiments pertain to methods of use and making the composition. In several embodiments, the nanoparticle compositions comprise one or more active agents (e.g., single active agents or combinations thereof). In several embodiments, the composition is comprised of high-quality, pure, and/or high-grade ingredients (e.g., highly pure) that yield a well-characterized, reproducible delivery system (e.g., comprising mixed nanoparticles). In several embodiments, the compositions have enhanced stability (e.g., are stable for long periods of time under various conditions). In several embodiments, the composition confers water solubility to hydrophobic agents, to combinations of hydrophobic agents, and/or to combinations of hydrophobic and hydrophilic agents. In several embodiments, the nanoparticle composition comprises a liposomal and/or nano-emulsion composition of an active agent.

While some embodiments are disclosed herein in relation to particular active agents, it is to be understood that other active agents, nutrients, and/or combinations thereof can be employed in the compositions disclosed herein. In several embodiments, for example, hydrophilic active agents may also be provided in the disclosed nanoparticle compositions (e.g., alone, in combination with other hydrophilic active agents, and/or in combination with hydrophobic active agents). Advantageously, the compositions disclosed herein may enhance the delivery of and/or slow or lessen the degradation of hydrophilic or hydrophobic agents (or combinations thereof). Additionally, while some embodiments are disclosed in relation to nanoparticles (e.g., mixed micelle-based nanoparticles), as disclosed elsewhere herein, microparticles are also envisioned.

In some embodiments, as disclosed elsewhere herein, a lipid-based particle composition is provided to aid in the delivery of therapeutic agents. In some embodiments, the lipid-based particle composition (e.g., when in water or dried) comprises multilamellar particle vesicles, unilamellar particle vesicles, and/or emulsion particles. In some embodiments, the composition is characterized by having multiple types of particles (e.g., multilamellar, unilamellar, emulsion, etc.). In some embodiments, a majority of the particles present are emulsion particles. In some embodiments, a majority of the particles present are lamellar (multilamellar and/or unilamellar). In some embodiments, a majority of the particles are unilamellar. In some embodiments, a minority of the particles present are emulsion particles. In some embodiments, a minority of the particles present are multilamellar.

In some embodiments, of the particles present in the composition (e.g., the aqueous composition), equal to or less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 75%, 85%, or 95%, (or ranges spanning and/or including the aforementioned values) are multilamellar nanoparticle vesicles. In some embodiments, of the particles present in the composition (e.g., the aqueous composition), equal to or at least about 1%, 2%, 3%, 4%, 5%, 6%, 7%, 8%, 9%, 10%, 11%, 12%, 13%, 14%, or 15% (or ranges spanning and/or including the aforementioned values) are multilamellar nanoparticle vesicles. For example, in some embodiments, between about 5% and about 10% of the particles present are multilamellar. In some embodiments, of the particles present more are unilamellar than are multilamellar.

In some embodiments, of the particles present in the composition (e.g., the aqueous composition), equal to or at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% (or ranges spanning and/or including the aforementioned values) are unilamellar nanoparticle vesicles. In some embodiments, of the particles present in the composition (e.g., the aqueous composition), equal to or at least about 50%, 55%, 60%, 65%, 70%, 75%, 80%, 85%, or 90% (or ranges spanning and/or including the aforementioned values) are unilamellar nanoparticle vesicles. For example, in some embodiments, between about 50% and 100%, or 50% and 90%, or 50% and 80%, or 50% and 70%, or 50% and 60%, of the particles present are unilamellar. In some embodiments, more of the particles present are unilamellar when compared to multilamellar. In some embodiments, a majority of the particles are unilamellar.

In some embodiments, of the particles present in the composition (e.g., the aqueous composition), equal to or less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 75%, 85%, 95%, or 100% (or ranges spanning and/or including the aforementioned values) are emulsion particles. In some embodiments, of the particles present in the composition (e.g., the aqueous composition), equal to or less than about 1%, 2%, 3%, 4%, 5%, 6%, 7%, 8%, 9%, 10%, 11%, 12%, 13%, 14%, 15%, 16%, 17%, 18%, 19%, or 20%, (or ranges spanning and/or including the aforementioned values) are emulsion particles. For example, in some embodiments, between about 1% to about 20%, or about 1% to about 10%, of the particles present are emulsion particles.

In some embodiments, liposomes comprise equal to or at least about 5%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% (or ranges spanning and/or including the aforementioned values) of the particles present in the composition (e.g., the aqueous composition).

In some embodiments, micelle particles comprise equal to or less than about 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1% (or ranges spanning and/or including the aforementioned values) of the particles present in the composition (e.g., the aqueous composition). In some embodiments, a composition does not comprise micelle particles.

In some embodiments, irregular particles comprise equal to or less than about 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1% (or ranges spanning and/or including the aforementioned values) of the particles present in the composition (e.g., the aqueous composition). In some embodiments, a composition does not comprise irregular particles.

In some embodiments, combined lamellar and emulsion particles comprise equal to or at least about 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition).

In some embodiments, mixed-micelle particles comprise equal to or less than about 95%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, or 1% (or ranges spanning and/or including the aforementioned values) of the particles present in the composition (e.g., the aqueous composition). In some embodiments, a composition does not comprise mixed-micelle particles.

In some embodiments, the particle compositions can comprise combinations of multilamellar particles, unilamellar particles, emulsion particles, micelle particles, irregular particles, and/or liposomes. In some embodiments, the particle compositions can comprise or consist of combinations of multilamellar particles, unilamellar particles, and emulsion particles.

The percentages and/or concentrations of particles present in the composition may be purposefully modified. In some embodiments, the percentage and/or concentration of the particles present in the composition are tailored to the active compound and/or the liquid comprising the particles. Such tailoring may lead to more homogenization and/or dispersion in the liquid. The tailoring may stabilize dispersion in the liquid. Such tailoring may also tailor to specific densities of the compositions. The densities of the compositions can be matched or different from a liquid that the compositions are contacted by or contained within.

In some embodiments, the composition is biased towards one type of particle, such as solid particles or liposomes. The composition may be biased by increasing or decreasing the ratio in the composition of lipids that are solid at room temperature to lipids that are liquid at room temperature. Biasing the composition may alter characteristics of the composition including density, particle composition, solubility, pharmacokinetic properties, or other characteristics described herein. In some embodiments, the composition is biased towards unilamellar liposomes by increasing the concentration of lipids that are solid at room temperature (e.g., phosphatidylcholine).

In some embodiments, the composition is free of surfactants other than the phosphatidylcholine and those in a plant extract. In some embodiments, the composition is free of synthetic surfactants. A synthetic surfactant may be a surfactant that is not produced in nature. In some embodiments, the nanoparticle composition does not comprise a surfactant other than a phospholipid, a saponin, and/or a surfactant that is contained in the plant extract. In some embodiments, a composition is free of emulsifiers at concentrations greater than about 1/10^{th} that of phosphatidylcholine and/or emulsifiers in the plant extract. In some embodiments, a composition comprises less than about 1.0, 2.0, 3.0, 4.0, 5.0, 6.0, 7.0, 8.0, 9.0, or 10.0 wt. % of a surfactant and/or emulsifiers other than phosphatidylcholine and those in the plant extract.

In some embodiments, a composition comprises high purity triglycerides, such as oleic acid and/or conjugated linoleics. In some embodiments, the composition does not comprise high purity triglycerides. In some embodiments, the composition does not comprise medium chain triglycerides. In some instances, the composition does not comprise an oil. In some instances, the composition does not comprise more than 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01 wt. % of an oil. The composition may be formulated, such as by changing the composition or concentration of lipids, for specific delivery or specific metabolism. For example, the composition may comprise medium chain triglycerides to bias the composition towards phase 1 liver metabolism. In some embodiments, the composition is formulated for a specific absorption mechanism, such as lymphatic absorption or liver first pass.

As disclosed elsewhere herein, in some embodiments, the nanoparticle composition comprises a lipid source. In several embodiments, the lipid source comprises a charged lipid, which can impart a charge to the nanoparticle. In several embodiments, the lipid source comprises a neutral lipid. In several embodiments, the lipid source comprises one or more phospholipids. In several embodiments, the one or more phospholipids comprises one or more of phosphatidic acid, phosphatidylethanolamine, phosphatidylcholine, phosphatidylserine, phosphatidylinositol, phosphatidylinositol phosphate, phosphatidylinositol bisphosphate, phosphatidylinositol trisphosphate, lipoid H 100-3™, phospholipon 90H™, phospholipon 80H™, lipoid 100-3™, lipoid P75-3™, or any combination of the foregoing. In several embodiments, the lipid source is a phosphatidylcholine. In several embodiments, the only lipid present is a phosphatidylcholine (e.g., the lipid source lacks phospholipids other than phosphatidylcholine or is substantially free of other phospholipids). In several embodiments, the one or more lipid source lipid(s) (collectively or individually) are present in the composition at a dry wt. % of equal to or less than about: 0%, 5%, 10%, 15%, 20%, 30%, 40%, 50%, 60%, 70%, 80%, 90%, or ranges including and/or spanning the aforementioned values. In several embodiments, the one or more lipid source lipid(s) (collectively or individually) are present in the composition at a wet wt. % of equal to or less than about: 0%, 0.1%, 0.5%, 1.0%, 2.5%, 4%, 5%, 6%, 7.5%, 10%, 12.5%, 15%, 17.5%, 20%, 25%, 30%, 35%, 40%, 45%, 50%, or ranges including and/or spanning the aforementioned values. In several embodiments, the one or more lipid source lipid(s) (collectively or individually) are present in the composition at a wet w/v of equal to or less than about: 0 mg/mL, 0.1 mg/mL, 0.5 mg/mL, 1.0 mg/mL, 2.5 mg/mL, 4 mg/mL, 5 mg/mL, 6 mg/mL, 7.5 mg/mL, 10 mg/mL, 12.5 mg/mL, 15 mg/mL, 17.5 mg/mL, 20 mg/mL, 25 mg/mL, 30 mg/mL, 35 mg/mL, 40 mg/mL, 45 mg/mL, 50 mg/mL, 100 mg/mL, 150 mg/mL, 200 mg/mL, 250 mg/mL, 300 mg/mL, 350 mg/mL, 400 mg/mL, 450 mg/mL, 500 mg/mL or ranges including and/or spanning the aforementioned values. In several embodiments, as disclosed elsewhere herein, the composition is aqueous, while in others it has been dried into a powder. For instance, as disclosed elsewhere herein, in several embodiments, the composition is aqueous (wet), while in others it has been dried into a powder (dry). In several embodiments, the one or more lipid(s) of the lipid source are synthetic, derived from sunflower, soy, egg, or mixtures thereof. In several embodiments, the one or more lipids of the lipid source can be hydrogenated or non-hydrogenated. In several embodiments, the lipid source exceeds requirements of the United States Pharmacopeia (is USP grade) and/or is National Formulary (NF) grade.

In several embodiments, the lipid source (e.g., phosphatidylcholine, including hydrogenated soybean phosphatidylcholine) may be of high purity. For example, in some embodiments, the phosphatidylcholine includes over 96.3% phosphatidylcholine (hydrogenated) or over 99% phosphatidylcholine (hydrogenated) (PC100). In several embodiments, the one or more lipids of the lipid source has a purity of greater than or equal to about: 92.5%, 95%, 96%, 96.3%, 98%, 99%, 100%, or ranges including and/or spanning the aforementioned values. In several embodiments, the one or more lipids of the lipid source has a total % impurity content by weight of less than or equal to about: 8.5%, 5%, 4%, 3.7%, 2%, 1%, 0%, or ranges including and/or spanning the aforementioned values. In several embodiments, the one or more lipids of the lipid source comprises less than or equal to about 8.5%, 5%, 4%, 3.7%, 2%, 1%, or 0.1% (or ranges including and/or spanning the aforementioned values) of any one or more of saturated fatty acids, monounsaturated fatty acids, polyunsaturated fatty acids (C 18), arachidonic acid (ARA) (C 20:4), docosahexaenoic acid DHA (C 22:6), phosphatidic acid, phosphatidylethanolamine, and/or lysophosphatidylcholine by weight. In several embodiments, the one or more lipids of the lipid source has less than about 1.1% lysophosphatidylcholine and less than about 2.0% triglycerides by weight.

In some embodiments, the lipid source (e.g., phosphatidylcholine, including hydrogenated soybean phosphatidylcholine) may be 90%, 80%, 70%, 60%, 50%, 40%, 30%, 20% pure, or ranges including and/or spanning the aforementioned values. In some embodiments the lipid source has a maximum concentration of hydrogenated lipids equal to or less than about 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5%, and/or 0% of the lipid source. In some instances, the phospholipid has a maximum concentration of hydrogenated phospholipids equal to or less than about 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5%, and/or 0% of the phospholipids (e.g., phosphatidylcholine).

As disclosed elsewhere herein, in some embodiments, the nanoparticle composition comprises a surfactant or synthetic surfactant. In some embodiments, the nanoparticle composition does not comprise a synthetic surfactant. A synthetic surfactant may be a surfactant that is not produced in nature, such as polyoxyethylene sorbitan esters (e.g., polysorbates/tweens, including polysorbate 80, polysorbate 20, etc.), cremophor (e.g., a non-ionic solubilizer and emulsifier that is made by reacting ethylene oxide with castor oil), propylene oxide-modified polymethylsiloxane, dodecyl betaine, lauramidopropyl betaine, cocoamido-2-hydroxypropyl sulfobetaine, sodium stearate (or other stearate salts), polyoxyethylene alcohol, mono- and diglycerides of fatty acids (MDG), acetic acid esters of MDG, lactic acid esters of MDG, citric acid esters of MDG, mono- and diacetyl tartaric acid esters of MDG, sucrose esters of fatty acids, polyglycerol esters of fatty acids (e.g., polyglycerol esters), polyglycerol polyricinoleate, propane-1,2-diol esters of fatty acids, propylene glycol esters, sodium stearoyl-2-lactylate, calcium stearoyl-2-lactylate, sorbitan fatty acid esters, silicone emulsifiers, sorbitan trioleate, dioctyl sodium sulfosuccinate, dioctyl sodium sulfonate, polyoxyethylene, hydrogenated castor oil, sucrose fatty acid ester, polyethylene glycol, dextrans, such as cyclodextran. In some embodiments, the nanoparticle composition does not comprise a surfactant other than a phospholipid, a saponin, and/or a surfactant that is contained in the plant extract. In several embodiments, the surfactant is a pharmaceutically acceptable surfactant. In several embodiments, the surfactant is a food surfactant. In several embodiments, the surfactant comprises or does not comprise one or more of a polyoxyethylene sorbitan esters (e.g., polysorbates/tweens, including polysorbate 80, polysorbate 20, etc.), cremophor (e.g., a non-ionic solubilizer and emulsifier that is made by reacting ethylene oxide with castor oil), propylene oxide-modified polymethylsiloxane, dodecyl betaine, lauramidopropyl betaine, cocoamido-2-hydroxypropyl sulfobetaine, sodium stearate (or other stearate salts), polyoxyethylene alcohol, lecithins, mono- and diglycerides of fatty acids (MDG), acetic acid esters of MDG, lactic acid esters of MDG, citric acid esters of MDG, mono- and diacetyl tartaric acid esters of MDG, sucrose esters of fatty acids, polyglycerol esters of fatty acids (e.g., polyglycerol esters), polyglycerol polyricinoleate, propane-1,2-diol esters of fatty acids, propylene glycol esters, sodium stearoyl-2-lactylate, calcium stearoyl-2-lactylate, sorbitan fatty acid esters, quillaja extract surfactant, yucca extract surfactant, saponins, silicone emulsifiers, sorbitan trioleate, soya lecithin, dioctyl sodium sulfosuccinate, dioctyl sodium sulfonate, polyoxyethylene, hydrogenated castor oil, sucrose fatty acid ester, or combinations of any of the foregoing. Natural or synthetic surfactants can be used, including polyethylene glycol and dextrans, such as cyclodextran. In several embodiments, the one or more surfactants are present in the nanoparticle composition (collectively or individually) at a dry wt. % of equal to or less than about: 0%, 5%, 10%, 15%, 20%, 30%, 40%, 50%, 60%, or ranges including and/or spanning the aforementioned values. Surfactants can include or exclude in the nanoparticle composition cationic, anionic, non-ionic, and zwitterionic surfactants. In several embodiments, the one or more surfactants (collectively or individually) are present in the composition at a wet wt. % of equal to or less than about: 0%, 0.1%, 0.5%, 1.0%, 2.5%, 4%, 5%, 6%, 7.5%, 10%, 12.5%, 15%, 17.5%, or ranges including and/or spanning the aforementioned values. In several embodiments, the one or more surfactants (collectively or individually) are present in the composition at a wet w/v of equal to or less than about: 0 mg/mL, 0.1 mg/mL, 0.5 mg/mL, 1.0 mg/mL, 2.5 mg/mL, 4 mg/mL, 5 mg/mL, 6 mg/mL, 7.5 mg/mL, 10 mg/mL, 12.5 mg/mL, 15 mg/mL, 17.5 mg/mL, or ranges including and/or spanning the aforementioned values. In several embodiments, the surfactant exceeds requirements of the United States Pharmacopeia (is USP grade) and/or is National Formulary (NF) grade.

In several embodiments, one or more co-emulsifiers are used. In some embodiments, a co-emulsifier is not comprised in the nanoparticle composition. In several embodiments, the co-emulsifier is a pharmaceutically acceptable co-emulsifier. In several embodiments, the co-emulsifier is selected from the group consisting of, or excludes, oleic acid, miglyol 812N (all versions), triglycerides, conjugated linoleic acid (CLA), cetearyl olivate, isoprpyle myristate, glyceryl stearate (e.g., glycerol monostearate), celluloses and polysaccharides (e.g., methylcellulose, propylmethylcellulose, hydroxypropyl methylcellulose, xanthan gum, etc.) and/or combinations of any of the foregoing. In several embodiments, the one or more co-emulsifiers are present in the nanoparticle composition (collectively or individually) at a dry wt. % of equal to or less than about: 0%, 0.01%, 0.05%, 0.1%, 0.2%, 0.3%, 0.4%, 0.5%, 0.6%, 0.7%, 0.8%, 0.9%, 1%, 2%, 3%, 4%, 5%, 10%, 15%, 20%, 30%, 40%, 50%, 60%, or ranges including and/or spanning the aforementioned values. In several embodiments, the one or more co-emulsifiers (collectively or individually) are present in the composition at a wet wt. % of equal to or less than about: 0%, 0.01%, 0.05%, 0.1%, 0.2%, 0.3%, 0.4%, 0.5%, 0.6%, 0.7%, 0.8%, 0.9%, 1.0%, 2.5%, 4%, 5%, 6%, 7.5%, 10%, 12.5%, 15%, 17.5%, or ranges including and/or spanning the aforementioned values. In several embodiments, the one or more co-emulsifiers (collectively or individually) are present in the composition at a wet w/v of equal to or less than about: 0 mg/mL, 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 2.5 mg/mL, 4 mg/mL, 5 mg/mL, 6 mg/mL, 7.5 mg/mL, 10 mg/mL, 12.5 mg/mL, 15 mg/mL, 17.5 mg/mL, or ranges including and/or spanning the aforementioned values. In several embodiments, the co-emulsifiers exceeds requirements of the United States Pharmacopeia (is USP grade) and/or is National Formulary (NF) grade.

In some embodiments, the co-emulsifier component comprises or excludes a medium chain triglyceride (MCT) or a MCT-substitute. In some embodiments, the medium chain triglyceride comprises a fatty acid selected from one or more of caproic acid, octanoic acid, capric acid, caprylic acid, and/or lauric acid (e.g., is formed from). In some embodiments, the medium chain triglyceride comprises or excludes a fatty acid 6-12 carbons in length (e.g., 6, 7, 8, 9, 10, 11, or 12). In some embodiments, the co-emulsifier component comprises or excludes a long chain triglyceride (LCT). In some embodiments, the long chain triglyceride comprises or excludes a fatty acid greater than 12 carbons in length (e.g., greater than or equal to 13, 14, 15, 16, 17, 18, 19, or 20 carbons in length, or ranges including and/or spanning the aforementioned values). In some embodiments, the co-emulsifier component is a single lipid. In some embodiments, the co-emulsifier component is MCT. In some embodiments, the MCT is highly pure. In some embodiments, the MCT has a purity by weight % of equal to or greater than about: 90%, 95%, 97%, 98%, 99%, 100%, or ranges including and/or spanning the aforementioned values. In some embodiments, the MCT (or LCT) is present in the nanoparticle composition at dry weight % of equal to or greater than or less than about: 0%, 10%, 20%, 30%, 35%, 40%, 45%, 50%, or ranges including and/or spanning the aforementioned values. In some embodiments, the MCT-substitute lipid (e.g., the non-phospholipid lipid) is selected from one or more of a seed oil (sunflower oil. grapeseed oil, soybean oil), a vegetable oil (olive oil, palm oil, cocoa butter, coconut oil), an animal oil (lard, tallow, salmon oil, fish oil), oleic acid, capric acid, caprylic acid, and ethyl esters of triglycerides of such (Captex 8000™, Captex GTO™, Captex 1000™, glycerol monooleate, glycerol monostearate (Geleol™ Mono and Diglyceride NF), omega-3 fatty acids (α-linolenic acid (ALA), eicosapentaenoic acid (EPA), docosahexaenoic acid (DHA), Tonalin™, Pronova Pure® 46:38, free fatty acid Tonalin FFA 80™), conjugated linoleic acid, alpha glycerylphosphorylcholine (alpha GPC), palmitoylethanolamide (PEA), cetyl alcohol, or emulsifying wax. In some embodiments, the one or more MCT-substitute(s) are present in the nanoparticle composition (collectively or individually) at a dry wt. % of equal to or less than about: 0%, 0.5%, 1.0%, 2.5%, 5%, 10%, 15%, 20%, 30%, 40%, 50%, 60%, 80% or ranges including and/or spanning the aforementioned values. In some embodiments, the one or more MCT-substitute(s) (collectively or individually) are present in the composition at a wet wt. % of equal to or less than about: 0%, 0.5%, 1.0% 2.5%, 5%, 7.5%, 10%, 12.5%, 15%, 20%, 30%, 40%, 60% or ranges including and/or spanning the aforementioned values. In some embodiments, the MCT-substitute has a purity of greater than or equal to about: 70%, 80%, 85%, 92.5%, 95%, 96%, 98%, 99%, 99.9%, 100%, or ranges including and/or spanning the aforementioned values. In some embodiments, the MCT-substitute has a total % impurity content by weight of less than or equal to about: 8.5%, 5%, 4%, 3.7%, 2%, 1%, 0%, or ranges including and/or spanning the aforementioned values.

In some embodiments, the co-emulsifier component comprises or excludes one or more gums. In some embodiments, the gum is or excludes a xanthan gum and/or a biosaccharide gum, such as biosaccharide gum-1, -2, -3, -4, -5, etc. In some embodiments, the gum provides increased stability of the nanoparticle over time and/or at higher temperatures. Higher temperatures may include exposure of the nanoparticles to a hot liquid, such as a hot aqueous liquid such as a hot beverage (e.g., coffee, tea, hot chocolate, etc.). The higher temperatures may be temperatures such as 70° C. or higher. Higher temperatures may include above, at, below, between, or any range of 70° C. to 100° C., such as 70° C. to 85° C., 70° C. to 80° C., 70° C., 75° C., 80° C., 85° C., 95° C., or 100° C. In several embodiments, after a 30 minute period in a hot liquid, the particle size and/or PDI varies by less than or equal to about: 1%, 5%, 10%, 20%, 30%, or ranges including and/or spanning the aforementioned values. In several embodiments, after a 30 minute period in a hot liquid, the active agent (e.g., pharmaceuticals, nutraceuticals, cosmetics, pigments, flavorings, and the like) concentration drops by less than or equal to about: 1%, 5%, 10%, 15%, or ranges including and/or spanning the aforementioned values. In several embodiments, the one or more gums are present in the nanoparticle composition (collectively or individually) at a dry wt. % of equal to or less than about: 0%, 0.01%, 0.05%, 0.1%, 0.2%, 0.3%, 0.4%, 0.5%, 0.6%, 0.7%, 0.8%, 0.9%, 1%, 2%, 3%, 4%, 5%, or ranges including and/or spanning the aforementioned values. In several embodiments, the one or more gums (collectively or individually) are present in the composition at a wet wt. % of equal to or less than about: 0%, 0.01%, 0.05%, 0.1%, 0.2%, 0.3%, 0.4%, 0.5%, 0.6%, 0.7%, 0.8%, 0.9%, 1.0%, 2.5%, 4%, 5%, or ranges including and/or spanning the aforementioned values. In several embodiments, the one or more gums (collectively or individually) are present in the composition at a wet w/v of equal to or less than about: 0 mg/mL, 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 2.5 mg/mL, 4 mg/mL, 5 mg/mL, or ranges including and/or spanning the aforementioned values.

In some embodiments, the nanoparticle composition comprises or excludes one or more sterols. In some embodiments, the one or more sterols comprises or excludes one or more cholesterols, ergosterols, hopanoids, hydroxysteroids, phytosterols (e.g., VEGAPURE®), ecdysteroids, and/or steroids. In some embodiments the sterol comprises a cholesterol. In some embodiments, the sterol component is a single sterol. In some embodiments, the sterol component is cholesterol. In some embodiments, the cholesterol (or other sterol) is highly pure. In some embodiments, the one or more sterol(s) (e.g., cholesterol, and/or other sterols), collectively or individually, are present in the aqueous composition at a concentration of less than or equal to about: 50 mg/mL, 40 mg/mL, 20 mg/mL, 10 mg/mL, 5 mg/mL, 0 mg/ml or ranges including and/or spanning the aforementioned values. In some embodiments, the one or more sterol(s) are present in the composition at a dry wt. % of equal to or less than about: 0%, 0.25%, 0.5%, 1%, 5%, 7.5%, 10%, 15%, 20%, 25%, or ranges including and/or spanning the aforementioned values. In some embodiments, the one or more sterol(s) (collectively or individually) are present in the composition at a wet wt. % of equal to or less than about: 0%, 0.1%, 0.25%, 0.5%, 1%, 2%, 3%, 4%, 5%, 7.5%, 10%, or ranges including and/or spanning the aforementioned values. In some embodiments, the cholesterol used in the composition comprises or excludes cholesterol from one or more of sheep's wool, synthetic cholesterol, or semisynthetic cholesterol from plant origin. In some embodiments, the sterol has a purity of greater than or equal to about: 92.5%, 95%, 96%, 98%, 99%, 99.9%, 100.0%, or ranges including and/or spanning the aforementioned values. In some embodiments, the sterol has a total % impurity content by weight of less than or equal to about: 8.5%, 5%, 4%, 3.7%, 2%, 1%, 0%, or ranges including and/or spanning the aforementioned values. In some embodiments, the sterol is cholesterol. In some embodiments, the sterol is not cholesterol. In some embodiments, the sterol is or excludes a phytosterol.

In several embodiments, as disclosed elsewhere herein, the nanoparticle composition provides an oil-in-water emulsion (e.g., a nanoemulsion), water-in-oil emulsion, a water-in-oil-in-water emulsion, an oil-in-water-in-oil emulsion, a liposome (and variants including multi-lamellar, double liposome preparations, etc.), micelle, and/or solid lipid particles. Any one of these structures may be provided as a nanoparticle or microparticle.

### B. Active Component

Certain embodiments disclosed herein comprise nanoparticle products, which may comprise active compositions. In several embodiments, the composition comprises a nanoparticle delivery system, which may be utilized to impart apparent aqueous solubility and deliverability to an otherwise practically water insoluble molecule (e.g., hydrophobic pharmaceuticals, hydrophobic nutraceuticals, hydrophobic vitamins, hydrophobic extracts). Attributes of some embodiments disclosed herein have been determined to be high quality and reproducible. Such reproducibility and low variations may allow the products to generate a reproducible certificate of analysis for different batches.

In several embodiments, the compositions disclosed herein (e.g., mixed nanoparticle compositions and/or formulations comprising them) increase the bioavailability of active agents (e.g., pharmaceutical, nutraceutical, etc.), decrease the time for absorption of those active agents, increase the stability of the active agents or the particles comprising the active agents, increase the consistency of delivery (e.g., by limiting batch-to-batch variation), and/or increase the efficacy of the active agents (higher dosing and/or faster onset of activity).

As disclosed elsewhere herein, in some embodiments, the compositions (including the mixed nanoparticle compositions) disclosed herein are able to deliver active agents that are highly pure. In several embodiments, an active agent (e.g., pharmaceuticals, nutraceuticals, cosmetics, pigments, flavorings, and the like) formulated with the nanoparticle has a purity of greater than or equal to about: 4%, 10%, 20%, 30%, 40% 50%, 60%, 70%, 80%, 90%, 95%, 98%, 99%, 99.5%, 99.9%, 99.99%, or ranges including and/or spanning the aforementioned values.

In certain embodiments, Therapeutic Ingredients and Agents can be selected from those described in U.S. Pat. No. 11,260,033, filed Mar. 24, 2021, and granted Mar. 1, 2022, which is hereby incorporated in its entirety for the purposes described herein.

In some embodiments, at least one therapeutic agent in the lipid-based particle composition (and/or combination of therapeutic agents provided in the lipid-based particle composition) has a room temperature, neutral pH, and atmospheric pressure solubility in MCT oil of less than 1 mg/g. In some embodiments, at least one therapeutic agent in the lipid-based particle composition (and/or combination of therapeutic agents provided in the lipid-based particle composition) has a room temperature solubility in water at room temperature, neutral pH, and atmospheric pressure of less than 1 mg/g. In some embodiments, at least one therapeutic agent in the lipid-based particle composition (and/or combination of therapeutic agents provided in the lipid-based particle composition) is hydrophobic. In some embodiments, at least one therapeutic agent in the lipid-based particle composition (and/or combination of therapeutic agents provided in the lipid-based particle composition) is hydrophillic. In some embodiments, at least one therapeutic agent in the lipid-based particle composition (and/or combination of therapeutic agents provided in the lipid-based particle composition) is hydroneutral. In some embodiments, at least one therapeutic agent used to prepare a lipid-based particle composition has an aqueous solubility of less than or equal to about: 0.05 mg/ml, 0.01 mg/ml, 0.012 mg/ml, 0.001 mg/ml, or ranges including and/or spanning the aforementioned values in pure water at room temperature, neutral pH, and atmospheric pressure.

As disclosed herein, some embodiments relate to delivery systems (e.g., mixed nanoparticle compositions and/or formulations comprising the same) that improve the absorption of the highly insoluble forms of an active agent (e.g., pharmaceuticals, nutraceuticals, cosmetics, pigments, flavorings, and the like) or combination of agents. In several embodiments, the active agent encapsulated in the nanoparticle compositions disclosed herein (e.g., the starting material) has an aqueous solubility of less than or equal to about: 0.05 mg/mL, 0.01 mg/mL, 0.012 mg/mL, 0.001 mg/mL, or ranges including and/or spanning the aforementioned values, such as a range between 0.05 mg/mL to 0.001 mg/mL. In several embodiments, where a combination of active agents is encapsulated in the nanoparticle compositions disclosed herein, one or more or all of the active agents in the composition may have an aqueous solubility of less than or equal to about: 0.05 mg/mL, 0.01 mg/mL, 0.012 mg/mL, 0.001 mg/mL, or ranges including and/or spanning the aforementioned values, such as a range between 0.05 mg/mL to 0.001 mg/mL. In several embodiments, the aqueous solubility of the active agent or agents (and/or the amount of active agent or agents that can be provided in an aqueous solution) can be improved to equal to or to greater than about: 1 mg/mL, 5 mg/mL, 10 mg/mL, 20 mg/mL, 30 mg/mL, 50 mg/mL, 100 mg/mL, or ranges including and/or spanning the aforementioned values. For example, the aqueous solubility of the active agent or agents may be increased to 1 mg/mL to 50 mg/mL, 10 mg/mL to 100 mg/mL, 1 mg/mL to 20 mg/mL, etc.

In several embodiments, at least one active agent in the nanoparticle composition (and/or combination of active agents provided in the mixed nanoparticle composition) is hydrophobic. In several embodiments, at least one hydrophobic active agent used to prepare a nanoparticle composition as disclosed herein (e.g., pharmaceuticals, nutraceuticals, cosmetics, pigments, flavorings, and the like) has an aqueous solubility of less than or equal to about: 0.05 mg/mL, 0.01 mg/mL, 0.012 mg/mL, 0.001 mg/mL, or ranges including and/or spanning the aforementioned values. In several embodiments, the solubility of the at least one active agent (e.g. the amount of the active agent that can be provided in an aqueous solution) used to prepare the compositions disclosed herein can be improved to equal to or to greater than about: 1 mg/mL, 5 mg/mL, 20 mg/mL, 30 mg/mL, 50 mg/mL, 100 mg/mL, or ranges including and/or spanning the aforementioned values, when formulated with a nanoparticle. In several embodiments, the solubility of the at least one active agent can be improved by at least about: 50%, 100%, 150%, 200%, 500%, 1000%, 10,000%, or ranges including and or spanning the aforementioned values. In several embodiments, the solubility is measured as an amount that can be suspended for longer than 30 days and/or that can be dissolved in an aqueous solution at a concentration of at least 1 mg/mL.

In some embodiments, as disclosed herein, the therapeutic agent(s) is or may be synthetic. In some embodiments, as disclosed herein, the therapeutic agent(s) is or may be non-synthetic. In some embodiments, as disclosed herein, the therapeutic agent(s) is or may be semi-synthetic (e.g., prepared through fermentation, etc.). In some embodiments, as disclosed herein, the therapeutic agent(s) is or may be a plant and/or fungal extract. In some embodiments, the therapeutic agent(s) is or may comprise a polyphenol. In some embodiments, the therapeutic agents(s) is or may comprise a flavonoid. In some embodiments, the therapeutic agent(s) is or may comprise xanthohumol. In some embodiments, the therapeutic agent(s) is or may comprise resveratrol. In some embodiments, the therapeutic agent(s) is or may comprise dihydromyricetin. In some embodiments, the therapeutic agent(s) is or may comprise mangiferin. In some embodiments, the therapeutic agent(s) is or may comprise ascorbic acid or a salt of ascorbic acid. In some embodiments, as disclosed herein, the therapeutic agent(s) is or may be a vitamin. In some embodiments, the therapeutic agent(s) is or may comprise vitamin D3 (e.g., cholecalciferol).

In some embodiments, the therapeutic ingredient is or may be a combination of synthetic and non-synthetic therapeutic agents. In some embodiments, as disclosed elsewhere herein, the therapeutic ingredient is a single compound (or is substantially pure single compound). In some embodiments, the therapeutic ingredient comprises a mixture of different compounds (e.g., comprises a full spectrum of compounds from an extract, a mixture of isolates, etc.). In some embodiments, the therapeutic ingredient is an extract or a mixture of extracts from one or more therapeutic agent sources. In some embodiments, the therapeutic ingredient is a distillate or a mixture of distillates from one or more therapeutic agent sources.

In some embodiments, the therapeutic ingredient is a vitamin, nutrient, plant extract, nutraceutical, pharmaceutical, or other beneficial agents for delivery. In some instances, the therapeutic ingredient may be an analgesic, an anesthetic, an antibacterial agent, an anticonvulsant, an antidementia agent, an antidepressant, an antidote, a deterrent, a toxicologic agent, an antiemetic, an antifungal, an antigout agent, an anti-inflammatory agent, an antimigraine agent, an antimyasthenic agent, an antineoplastic agent, an antiparasitic agent, an antiparkinson agent, an antipsychotic, an antipasticity agent, an antiviral, an anxiolytic, a bipolar agent, a blood glucose regulator, a blood product, a blood modifier, a blood volume expander, a cardiovascular agent, a central nervous system agent, a dental agent, an oral agent, a dermatological agent, an enzyme replacement agent, an enzyme modifying agent, a gastrointestinal agent, a genitourinary agent, a hormonal agent, a hormone stimulant, a hormone replacement, a hormone modifying agent, a hormone suppressant, an immunological agent, an inflammatory bowel disease agent, a metabolic bone disease agent, an ophthalmic agent, an otic agent, a respiratory tract agent, a sedative, a hypnotic, a skeletal muscle relaxant, a therapeutic nutrient, a therapeutic mineral, and/or a therapeutic electrolyte. The hormonal agent, a hormone stimulant, a hormone replacement, a hormone modifying agent, and/or hormone suppressant may act on the adrenal system, the pituitary system, the prostaglandin system, sex hormone, the thyroid, and/or the parathyroid.

In some embodiments, the therapeutic ingredient may comprise a small molecule. In some embodiments, the therapeutic ingredient may comprise a biologic. In some embodiments, the therapeutic ingredient may comprise a biomolecule. In some embodiments, the therapeutic ingredient may comprise a macromolecule. In some embodiments, the therapeutic ingredient may comprise a nucleic acid, a protein, a lipid, a carbohydrate, or a combination thereof. In some embodiments, the therapeutic ingredient may comprise a cell or a derivative of a cell. In some embodiments, the therapeutic ingredient may comprise antisense RNA. In some embodiments, the therapeutic ingredient may comprise an siRNA, a miRNA, a lncRNA, or a combination thereof. In some embodiments, the therapeutic ingredient may comprise a nucleic acid vector.

In some embodiments, the active compound is one or more of a cannabinoid, cannabidiol, cannabigerol, cannabinol, cannabichromene, tetrahydrocannabivarin, tetrahydrocannabinol, full extracts of hemp, specific ratios of isolated cannabinoids, cannabigerolic acid, cannabidolic acid, mitragynine, payantheine, mitraphylline, speciociliantine, speciogynine, cholecalciferol, ergocalciferol, D,L-alpha-tocopherol, menaquinone, ascorbyl palmitate, retinyl palmitate, beta-sitosterol, plant sterol rich extracts, cholesterol, ubiquinone, phosphatidylcholine, phosphatidylinositol, phosphatidylethanolamine, phosphatidylserine, eicosapentaenoic/docosahexaenoic acid mixtures, oleic acid, conjugated linoleic acid, capric triglycerides, caprylic triglycerides, capric and caprylic triglyceride mixtures, peppermint, orange, lemon oils, lutein, kavain, methysticin, yangonin, dihydromethysticin, coenzyme Q10, a vitamin E, vitamin A, a beta carotene, vitamin D2, squalene, a vitamin K, a docosahexaenoic acid, a curcuminoid, a phytoceramide, vitamin D3, an ashwagandha extract, or any combination thereof.

In some embodiments, a therapeutic ingredient may comprise an active compound (e.g., therapeutic agent) selected from the group comprising a Flavonoid, Dihydromyrciten, a Vitamin, CoQ10, an Omega 3 fatty acid, Huperzine A, Bacopa monnieri extract, Cannabidiol, Cannabigerol, Cannabinol, Cannabichromene, Tetrahydrocannabivarin, Tetrahydrocannabinol, Full extracts of hemp, Specific ratios of isolated cannabinoids, Cannabigerolic acid, Cannabidolic acid, Mitragynine, Payantheine, Mitraphylline, Speciociliantine, Speciogynine, Cholecalciferol, Ergocalciferol, D,L-Alpha-Tocopherol, Menaquinone, Ascorbyl palmitate, Retinyl palmitate, Beta-Sitosterol, Plant Sterol Rich Extracts, Cholesterol, Ubiquinone, Phosphatidylcholine, Phosphatidylserine, Eicosapentaenoic/Docosahexaenoic Acid Mixtures, Oleic Acid, Conjugated Linoleic Acid, Capric Triglycerides, Caprylic Triglycerides, Capric and Caprylic Triglyceride mixtures, Peppermint, Orange, Lemon Oils, Lutein, Kavain, Methysticin, Yangonin, Dihydromethysticin, and/or derivatives thereof.

In some embodiments, when formulated, the dry weight % of one or more therapeutic agents present in the composition is equal to or at least about: 0.001%, 0.005%, 0.01%, 0.05%, 0.1%, 0.5%, 1%, 5%, 7.5%, 10%, 15%, 20%, 25%, 50%, 60%, 70%, or ranges including and/or spanning the aforementioned values. In some embodiments, the therapeutic agents are provided in an aqueous composition. In some embodiments, the wet weight % of the one or more therapeutic agents present in the composition (with water included) is equal to or at least about: 0.001%, 0.005%, 0.01%, 0.05%, 0.1%, 0.5%, 0.75%, 1%, 1.5%, 2%, 3%, 4%, 5%, 7.5%, 10%, 15%, 20%, 25%, or ranges including and/or spanning the aforementioned values. In some embodiments, the one or more therapeutic agents may be provided in the wet composition at a concentration of greater than or equal to about: 0.01 mg/ml, 0.05 mg/ml, 0.1 mg/ml, 0.5 mg/ml, 1 mg/ml, 5 mg/ml, 20 mg/ml, 30 mg/ml, 50 mg/ml, 100 mg/ml, 150 mg/ml, 200 mg/ml, or ranges including and/or spanning the aforementioned values.

In some embodiments, the one or more therapeutic agents used in the lipid-based particle compositions as disclosed herein has high purity as indicated by its existing in a solid form (e.g., powder) prior to processing (e.g., formulation into a composition as disclosed herein). In some embodiments, using the combinations disclosed herein, a composition comprising one or more therapeutic agents in an aqueous solution is provided. In some embodiments, the delivery system may be lipid-based and forms an oil-in-water emulsion (e.g., a nanoemulsion), a liposome, and/or solid lipid particle (e.g., nanoparticle). In some embodiments, the lipid-based delivery system provides particles in the nano-measurement range (as disclosed elsewhere herein). In some embodiments, a solid lipid nanoparticle is spherical or substantially spherical nanoparticle. In some embodiments, a solid lipid nanoparticle possesses a solid lipid core matrix that can solubilize lipophilic molecules. In some embodiments, the lipid core is not stabilized by surfactants and/or emulsifiers beyond a phospholipid, a saponin, and/or a surfactant that is contained in the plant extract. In some embodiments, surfactants are absent. In some embodiments, the size of the particle is measured as a mean diameter. In some embodiments, the size of the particle is measured by dynamic light scattering. In some embodiments, the size of the particle is measured using a zeta-sizer. In some embodiments, the size of the particle can be measured using Scanning Electron Microscopy (SEM). In some embodiments, the size of the particle is measured using a cryogenic SEM (cryo-SEM). Where the size of a nanoparticle is disclosed elsewhere herein, any one or more of these instruments or methods may be used to measure such sizes.

In some embodiments, as disclosed elsewhere herein, the lipid-based particle and/or nanoparticle composition, or simply the composition for brevity, comprises a therapeutic agent or combination of therapeutic agents, a plant extract, and one or more phospholipid. In some embodiments, as disclosed elsewhere herein, the composition comprises, consists of, or consists essentially of therapeutic agent or combination of therapeutic agents, a phospholipid, a plant extract, an optional bulking agent, an optional buffer, an optional solvent, and/or an optional preservative. In some embodiments, the composition is aqueous (e.g., contains water) while in other embodiments, the composition is dry (lacks water or substantially lacks water). In some embodiments, the composition has been dried (e.g., has been subjected to a process to remove most or substantially all water). In some embodiments, the composition comprises nanoparticles in water (e.g., as a solution, suspension, or emulsion). In other embodiments, the composition is provided as a powder (e.g., that can be constituted or reconstituted in water). In some embodiments, as disclosed elsewhere herein, the water content (in wt %) of the composition is less than or equal to about: 25%, 20%, 15%, 10%, 5%, 2.5%, 1%, 0.5%, 0.1%, or ranges including and/or spanning the aforementioned values.

As disclosed elsewhere herein, in some embodiments, the lipid-based particle composition may include one or more therapeutic agents (e.g., a single therapeutic agent or a combination of therapeutic agents) as a therapeutic ingredient. In some embodiments, the lipid-based particle composition may include a single therapeutic agent or a plurality of therapeutic agents (e.g., 1, 2, 3, 4, or more). In some embodiments, the lipid-based particle composition may include a single therapeutic extract or a plurality of therapeutic extracts (e.g., 1, 2, 3, 4, or more).

In some embodiments, the therapeutic agents, collectively or individually, are present in the aqueous lipid-based particle composition at a concentration of less than or equal to about: 250 mg/mL, 200 mg/ml, 150 mg/mL, 100 mg/ml, 75 mg/ml, 50 mg/ml, 25 mg/ml, 20 mg/ml, 10 mg/ml, 5 mg/ml, 2.5 mg/ml, 1 mg/ml, 0.5 mg/ml, 0.1 mg/ml, 0.01 mg/ml, 0.001 mg/ml, or ranges including and/or spanning the aforementioned values. In some embodiments, the one or more therapeutic agents, collectively or individually, are present in the aqueous composition at a concentration of greater than or equal to about: 250 mg/mL, 200 mg/ml, 150 mg/mL, 100 mg/ml, 75 mg/ml, 50 mg/ml, 25 mg/ml, 20 mg/ml, 10 mg/ml, 5 mg/ml, 2.5 mg/ml, 1 mg/ml, 0.5 mg/ml, 0.1 mg/ml, 0.01 mg/ml, 0.001 mg/ml, or ranges including and/or spanning the aforementioned values. In some embodiments, the one or more therapeutic agents, collectively or individually, are present in the composition at a dry wt % of equal to or at least about: 0.001%, 0.01%, 0.05%, 0.1%, 0.5%, 1%, 5%, 7.5%, 10%, 15%, 20%, 25%, 50%, 60%, 70%, or ranges including and/or spanning the aforementioned values. In some embodiments, the one or more therapeutic agents, collectively or individually, are present in the composition at a wet wt % of equal to or at least about: 0.01%, 0.05%, 0.1%, 0.25%, 0.5%, 1%, 2%, 3%, 4%, 5%, 7.5%, 10%, 15%, 20%, 25%, or ranges including and/or spanning the aforementioned values. In some embodiments, as disclosed elsewhere herein, the composition is aqueous, while in others it has been dried into a powder (that is free of or substantially free of water). In some embodiments, where the composition has been dried, it comprises a water content of less than or equal to 20%, 15%, 10%, 7.5%, 5%, 2.5%, 1%, or ranges including and/or spanning the aforementioned values.

In some embodiments, the therapeutic ingredient may comprise, consist of, or consist essentially of a full spectrum or broad spectrum plant/fungal extract. In some embodiments, the therapeutic ingredient may comprise, consist of, or consist essentially of a specific highly pure plant/fungal extract (e.g., a flavonoid, e.g., a prenylated flavonoid, e.g., xanthohumol). In some embodiments, a therapeutic ingredient may improve the functionality of a lipid-particle described herein.

In some embodiments, the therapeutic agent is one or more of a vitamin, a nutrient, a plant extract, a nutraceutical, a pharmaceutical, or another beneficial agent. In some embodiments, the therapeutic agent is hydrophilic. In some embodiments, the therapeutic agent is hydrophobic. In some embodiments, the therapeutic agent (e.g., non-cannabinoid therapeutic) is amphiphilic.

In several embodiments, the active ingredients provided in the nanoparticle composition may comprise, in addition to the plant extract of the composition, another extract that is an active agent.

In several embodiments, as disclosed elsewhere herein, a nanoparticle composition (e.g., a mixed micelle composition, a liposomal composition, solid lipid particles, oil-in-water emulsions, water-in-oil-in-water emulsions, water-in-oil emulsions, oil-in-water-in-oil emulsions, etc.) is provided to aid in the delivery of active agents. As disclosed elsewhere herein, in several embodiments, the nanoparticles comprise one or more active agents. In several embodiments, one or more of the active ingredients is a nutraceutical. In several embodiments, a composition comprising the nanoparticles disclosed herein comprises a therapeutically effective amount of one or more active ingredients. In several embodiments, the one or more active compounds comprise coenzyme Q10, a vitamin E, vitamin A, a beta carotene, vitamin D2, squalene, a vitamin K, a docosahexaenoic acid, a curcuminoid, a phytoceramide, vitamin D3, an ashwagandha extract, or a combination of any of the foregoing.

In several embodiments, the active ingredients provided in the nanoparticle composition may comprise, a plant extract that is an active agent. The plant extracts can be an unenriched extract (e.g., a mixture of agents as extracted from a single plant source), an enriched extract that has been enriched through purification processes (to have larger amounts of certain active agents), or any individual active component of the extract (e.g., a pure or substantially pure compound). As an example, the nanoparticle composition may include an unenriched extract that is isolated by bulk extraction of multiple actives from a plant biomass at one time. Alternatively, the nanoparticle composition may include actives that have been further processed to enrich the extract for particular active agents (e.g., having a higher wt. % of the active agent than un-processed extract). In several embodiments, alternatively, an active from an extract may be purified and may be pure and/or substantially pure, as disclosed elsewhere herein. For brevity, when a composition or particle disclosed herein is described as including plant extracts, the term “extracts” is meant to include any of the foregoing (e.g., including a full plant extract or partial plant extract that has not been enriched, an extract that has been enriched for particular components (e.g., particular active agents), and/or an extract that has been purified to provide, for example, highly pure individual components). In some instances, the plant extract is enriched in glycosides by the extraction. In some instances, the plant extract comprises at least, at most, between, at, or any range thereof of 10, 15, 20, 25, 30, 35, 40, 45, 50, 55, 60, 65, 70, 75, 80, 85, 90, 95, 98, or 99 wt. % glycosides. In some instances, the plant extract is enriched in terpenoid glycosides. In some instances, the plant extract comprises at least, at most, between, at, or any range thereof of 10, 15, 20, 25, 30, 35, 40, 45, 50, 55, 60, 65, 70, 75, 80, 85, 90, 95, 98, or 99 wt. % terpenoid glycosides. In some instances, the plant extract is a de-oiled plant extract. In some instances, the plant extract has had at least 50% of the oil removed, 60% removed, 70% removed, 80% removed, 90% removed, 95% removed, 98% removed, 99% removed, 100% removed, etc. In some instances, the plant extract comprises an extract of Tribulus terrestris, Yucca schidigera, Quillaja saponaria, Camillia sinesis, and/or Glycyrrhiza glabra.

Tribulus terrestris, also known as puncture vine or caltrop, is a taprooted herbaceous annual plant adapted to dry and temperate climates. In some instances, Tribulus terrestris extract is commercially available. Yucca schidigera, also known as Mojave yucca, is a flowering evergreen tree native to North American deserts. In some instances, Yucca schidigera extract is commercially available. Quillaja saponaria, also known as quillaja or soapbark, is an evergreen tree native to Chile. In some instances, Quillaja saponaria extract is commercially available. Camillia sinesis, also known as tea plant, tea shrub, or tea tree, is an evergreen shrub commonly used to produce black and green tea. In some instances, Camillia sinesis extract is commercially available. Glycyrrhiza glabra, also known as licorice, is an herbaceous perennial legume used in consumables, pharmaceuticals, cosmetics, and medicine. In some instances, Glycyrrhiza glabra extract is commercially available.

In some embodiments, the therapeutic agent is selected from the group consisting of Noopept (N-phenylacetyl-L-prolyglygice ethyl ester), a flavonoid, a vitamin (e.g., vitamin D, e.g., vitamin D3), melatonin, glutathione, gamma-glutamylcysteine (GGC), gamma-aminobutyric acid (GABA), valerian root, magnesium, theanine, 5-HTP, tyrosine, taurine, zinc, alpha fenchone, alpha terpinene, alpha terpineol, beta caryophyllene, alpha pinene, beta pinene, bisabolene, bisabolol, borneol, eucalyptol, gamma terpinene, guaiacol, humulene, linalool, myrcene, para cymene, phytol, terpinolene, limonene, others, and/or combinations thereof. In some embodiments, when a hydrophilic composition is used, it is mixed with the aqueous soluble ingredients before mixing with the lipid ingredients.

In some embodiments the lipid particles comprise extracts of mushrooms (e.g., cordyceps, lion mane, reishi, chaga gano, psilocybin (including the compound itself, natural extract forms, synthetic forms, derivatives of psilocybin, and prodrugs of any one of the foregoing), others, and/or combinations of any of the foregoing), kratom extracts, Kanna extracts, kava extracts, or combinations of any one or more of the foregoing.

In some embodiments, the compounds (e.g., therapeutic agents) are derived from or are broad spectrum extracts (e.g., oils, etc.), full spectrum extracts (e.g., oils, etc.), distillates (e.g., oils, etc.), and/or combinations thereof. In some embodiments, the lipid particles are composed of or comprise compounds (e.g., therapeutic agents) from a crude extract (an extract that is not further purified). In some embodiments, the lipid particle is composed of compounds (e.g., therapeutic agents) from combinations of sources.

In some embodiments, the active compound is encapsulated by particle at a concentration of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 mg, or ranges including and/or spanning the aforementioned values, per kg of the nanoparticle and/or a composition comprising the nanoparticle.

In several embodiments, when formulated, the dry weight % of one or more active agents (e.g., pharmaceuticals, nutraceuticals, cosmetics, pigments, flavorings, etc.) present in the nanoparticle compositions is equal to, between, at most, or at least about: 0.0015%, 0.002%, 0.005%, 0.01%, 0.05%, 0.1%, 0.5%, 1%, 5%, 7.5%, 10%, 12.5%, 15%, 20%, 25%, 30%, 35%, 40%, 45%, 50%, or ranges including and/or spanning the aforementioned values. In several embodiments, the active agents are provided in an aqueous composition. In several embodiments, the wet weight % of one or more active agents (e.g., pharmaceuticals, nutraceuticals, cosmetics, pigments, flavorings, etc.) present in the composition (with water included) is equal to, between, at most, or at least about: 0.001%, 0.005%, 0.01%, 0.05%, 0.1%, 0.5%, 1%, 2%, 3%, 4%, 5%, 7.5%, 10%, 12.5%, 15%, 17.5%, 20%, or ranges including and/or spanning the aforementioned values. In several embodiments, the one or more active agents (e.g., pharmaceuticals, nutraceuticals, cosmetics, pigments, flavorings, etc.) may be provided in the wet composition at a concentration of greater than, less than, between, or equal to about: 0.001 mg/mL, 0.005 mg/mL, 0.01 mg/mL, 0.05 mg/mL, 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 20 mg/mL, 30 mg/mL, 50 mg/mL, 100 mg/mL, 150 mg/mL, 200 mg/mL, 250 mg/mL, 300 mg/mL, 350 mg/mL, 400 mg/mL, 450 mg/mL, 500 mg/mL or ranges including and/or spanning the aforementioned values.

In some embodiments, the active compound is encapsulated by a nanoparticle at a concentration of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 200 mg, 250 mg, 300 mg, 350 mg, 400 mg, 450 mg, 500 mg, 1 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, 30 g, 35 g, 40 g, 45 g, 50 g, 55 g, 60 g, 65 g, 70 g, 75 g, 80 g, 85 g, 90 g, 95 g, 100 g, 125 g, 150 g, 175 g, 200 g, 250 g, 300 g, 350 g, 400 g, 450 g, 500 g, 600 g, 700 g, or ranges including and/or spanning the aforementioned values, per kg of the nanoparticle.

In several embodiments, as disclosed elsewhere herein, the nanoparticle (or compositions comprising the nanoparticle) may be used to deliver a combination of active ingredients (e.g., 1, 2, 3, 4, or more). In several embodiments, the composition comprises combinations of active compounds of varying ratios. For example, a first active compound to a second active compound present in the composition may be about: 100:1, 90:1, 80:1, 70:1, 60:1, 50:1, 40:1, 30:1, 20:1, 10:1, 5:1, 4:1, 3:12:1, 1:1, 1:2, 1:3, 1:4, 1:5, 1:10, 1:20, 1:30, 1:40, 1:50, 1:60, 1:70, 1:80, 1:90, 1:100, or ratios including and/or spanning the aforementioned ratios.

### C. Saponin

In some aspects, the saponin comprises one or more of a saponin with a triterpene, a steroid (such as spirostanol or furostanol), a steroidal alkaloid, and/or a acyclic carbon chain base structure. In some aspects, one or more of the saponin(s) comprise(s) one, two, or three monosaccharide unit. In some aspects, one or more of the saponin(s) comprise(s) a carbon chain. In some aspects, the saponin comprise one or more saponin(s) of the class dammaranes, tirucallanes, lupanes, hopanes, oleananes, taraxasteranes, ursanes, cycloartanes, lanostanes, cucurbitanes, and/or steroids. In some instances, one or more saponin is comprised in a plant, bacteria, fugi, or animal extract. In some instances, the extract comprises at least, at most, between, at, or any range thereof of 10, 15, 20, 25, 30, 35, 40, 45, 50, 55, 60, 65, 70, 75, 80, 85, 90, 95, 98, or 99 wt. % of one or more saponin.

In several embodiments, a saponin, a plant extract, compound isolated from a plant extract, and/or an active agent may be provided in a salt form. In several embodiments, salt is a pharmaceutically acceptable salt. In several embodiments, the salt is the acetate or citrate salt. In several embodiments, the composition may comprise mixtures of salt forms.

### D. Antioxidants and Essential Oils

In some embodiments, the active compound may be prepared using the thoroughness and diligence of pharmaceutical drug development to consumer products. In several embodiments, when a hydrophilic composition is used, it is mixed with the aqueous soluble ingredients before mixing with the lipid ingredients.

In some embodiments, the active compound comprises at least one cosmetic ingredient. The CTFA International Cosmetic Ingredient Dictionary and Handbook (2004 and 2008) describes a wide variety of non-limiting cosmetic ingredients that can be used in the context of the present disclosure, including as active compounds. In some embodiments, the active compound comprises a fragrance, flavor, dye, etc. Examples of these ingredient classes include: fragrance agents (artificial and natural; e.g., gluconic acid, phenoxyethanol, and triethanolamine), dyes and color ingredients (e.g., vegetable colorant, anthocyanins, purple sweet potato extract, black carrot extract, Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben, mushroom extract, etc.), pH adjusters (e.g., sodium hydroxide, ascorbic acid, and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate).

In some embodiments, an active compound comprises at least one antioxidant. Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

In some embodiments, the active compound comprises at least one moisturizing agent. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrrolidone carboxylic acid, potassium PCA, propylene glycol, saccharide isomerate, sodium glucuronate, sodium PCA, sorbitol, sucrose, trehalose, urea, and xylitol. Other examples include acetylated lanolin, acetylated lanolin alcohol, alanine, algae extract, Aloe barbadensis, Aloe barbadensis extract, Aloe barbadensis gel, Althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamom (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (Mentha piperita) oil, petrolatum, phospholipids, plankton extract, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

In some embodiments, the active compound comprises at least one essential oil. Essential oils include oils derived from herbs, flowers, trees, and other plants. Such oils are typically present as tiny droplets between the plant's cells, and can be extracted by several methods known to those of skill in the art (e.g., steam distilled, enfleurage, maceration, solvent extraction, or mechanical pressing). When these types of oils are exposed to air they tend to evaporate. As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160 to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang. Other essential oils known to those of skill in the art are also contemplated as being useful within the context of the present invention.

In some embodiments, the active agent comprises an algae extract. The algae extract may comprise ashwagandha and/or astoxantin.

In some embodiments, the active compound comprises a flavonoid. In some embodiments, the active compound comprises a prenylated (e.g., comprise a lipophilic prenyl side-chain) flavonoid. In some embodiments, a prenylated flavonoid is a chalcone, dihydrochalcone, flavone, flavanone, flavonol, and/or isoflavone. In some embodiments, a prenylated flavonoid comprises C-prenylation and/or O-prenylation. In some embodiments, a flavonoid may be a bioflavonoid, isoflavonoid, and/or neoflavonoid. In some embodiments, a flavonoid may be an anthocyanidin, chalcone, flavonol, flavanone, flavan-3-ol, flavanonol, flavone, and/or isoflavonoid.

In some embodiments, a chalcone may be, but is not limited to: xanthohumol, Flavokawin, Butein, Xanthoangelol, 4-Hydroxyderricin, Cardamonin, 2′,4′-Dihydroxychalcone, Isoliquiritigenin, Isosalipurposide, and/or Naringenin chalcone.

In some embodiments, a flavanol may be, but is not limited to: a Flava-3-ol, Catechin, Gallocatechin, Catechin 3-gallate, Gallocatechin 3-gallate, Epicatechins, Epigallocatechin, Epicatechin 3-gallate, Epigallocatechin 3-gallate, a Flavan-4-ol, a Flavan-3,4-diol, Leucoanthocyanidin, and/or a Proanthocyanidin.

In some embodiments, a flavone may be, but is not limited to: Luteolin, Apigenin, Tangeretin, a Flavanol, a Quercetin, Quercitrin, Rutin, a Kaempferol, Kaempferitrin, Astragalin, Naringenin, Sophoraflavonoloside, Myricetin, Fisetin, Isorhamnetin, Pachypodol, Rhamnazin, a Flavanone, Hesperetin, Hesperidin, Eriodictyol, Homoeriodictyol, a Flavanonol, Taxifolin, Dihydroquercetin, and/or Dihydrokaempferol.

In some embodiments, an Anthocyanidin may be, but is not limited to: Anthocyanidin, Cyanidin, Delphinidin, Malvidin, Pelargonidin, Peonidin, and/or Petunidin.

In some embodiments, an Isoflavonoid may be, but is not limited to: a Phytoestrogen, a Isoflavone, Genistein, Daidzein, Glycitein, an Isoflavane, Equol, Lonchocarpane, and/or Laxiflorane.

In some embodiments, a Neoflavonoid may be, but is not limited to: a Neoflavone, Calophyllolide, a Neoflavene, Dalbergichromene, Coutareagenin, Dalbergin, and/or Nivetin.

In some embodiments, a flavonoid is xanthohumol. In some embodiments, xanthohumol may be comprised in an extract that comprises one or more additional hop derived chemicals.

In some embodiments, the flavonoid is highly pure. In some embodiments, compositions provided herein comprise two or more sources of flavonoids. In some embodiments, compositions provided herein comprise two or more sources of prenylflavonoids. In some embodiments, the flavonoid has a purity of at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or ranges including and/or spanning the aforementioned values. In some embodiments, a flavonoid is about 70% to about 90% pure. In some embodiments, a flavonoid is at a concentration of about 300 to about 600 grams per liter in density. In some embodiments, a flavonoid comprises less than about, equal to about, or greater than about 40%, 39%, 38%, 37%, 36%, 35%, 34%, 33%, 32%, 31%, 30%, 29%, 28%, 27%, 26%, 25%, 24%, 23%, 22%, 21%, or 20% concentration of other prenylflavonoids.

In some embodiments, a flavonoid is greater than about 85%, 86%, 87%, 88%, 89%, 90%, 91%, 92%, 93%, 94%, or 95% pure. In some embodiments, a flavonoid is greater than about 90% pure. In some embodiments, a flavonoid is at a concentration of about 200 to 400 grams per liter in density. In some embodiments, a flavonoid comprises less than about, equal to about, or greater than about 5%, 6%, 7%, 8%, 9%, 10%, 11%, 12%, 13%, 14%, or 15% concentration of other prenylflavonoids.

In some embodiments, a flavonoid is about 70% to about 90% pure. In some embodiments, a flavonoid is at a concentration of about 300 to about 600 grams per liter in density. In some embodiments, a flavonoid comprises less than about, equal to about, or greater than about 40%, 39%, 38%, 37%, 36%, 35%, 34%, 33%, 32%, 31%, 30%, 29%, 28%, 27%, 26%, 25%, 24%, 23%, 22%, 21%, or 20% concentration of other prenylflavonoids.

In some embodiments, a flavonoid is greater than about 85%, 86%, 87%, 88%, 89%, 90%, 91%, 92%, 93%, 94%, or 95% pure. In some embodiments, a flavonoid is greater than about 90% pure. In some embodiments, a flavonoid is at a concentration of about 200 to 400 grams per liter in density. In some embodiments, a flavonoid comprises less than about, equal to about, or greater than about 5%, 6%, 7%, 8%, 9%, 10%, 11%, 12%, 13%, 14%, or 15% concentration of other prenylflavonoids.

In some embodiments, a flavonoid is about 65 to about 85% pure. In some embodiments, a flavonoid is at a concentration of about 150 to about 300 grams per liter in density. In some embodiments, a flavonoid comprises less than about, equal to about, or greater than about 40%, 39%, 38%, 37%, 36%, 35%, 34%, 33%, 32%, 31%, or 30% concentration of other prenylflavonoids. In some embodiments, a flavonoid is Isoxanthohumol. In some embodiments, a flavonoid is not Isoxanthohumol.

In some embodiments, a flavonoid comprises, consists essentially of, or consists of XanthoFlav™ by Hopsteiner (S.S. Steiner, Inc., NY, USA). In some embodiments, a xanthohumol comprises, consists essentially of, or consists of XanthoFlav™ by Hopsteiner (SS. Steiner, Inc., NY, USA). In some embodiments, a flavonoid comprises, consists essentially of, or consists of XanthoFlav™ Pure by Hopsteiner (S.S. Steiner, Inc., NY, USA). In some embodiments, a high purity xanthohumol comprises, consists essentially of, or consists of XanthoFlav™ Pure by Hopsteiner (SS. Steiner, Inc., NY, USA). In some embodiments, a flavonoid comprises, consists essentially of, or consists of Iso-XanthoFlav by Hopsteiner (S.S. Steiner, Inc., NY, USA). In some embodiments, the flavonoid xanthohumol is comprised in XanoHop™ and/or XanoHop™ Gold.

In some embodiments, the lipid-based particle composition comprises one or more one or more other additives, such as amino acids, polyethylene glycols, etc. In some embodiments, the one or more additives, collectively or individually, are present in the aqueous composition at a concentration of less than or equal to about: 400 mg/mL, 350 mg/mL, 300 mg/mL, 250 mg/mL, 200 mg/mL, 175 mg/mL, 150 mg/mL, 125 mg/mL, 100 mg/ml, 90 mg/ml, 80 mg/ml, 70 mg/ml, 60 mg/ml, 50 mg/ml, 40 mg/ml, 30 mg/ml, 20 mg/ml, 10 mg/ml, 5 mg/ml, 1.5 mg/ml, 1.2 mg/ml, 1 mg/ml, 0.9 mg/ml, 0.5 mg/ml, 0.1 mg/ml, 0.01 mg/ml, 0.001 mg/ml or ranges including and/or spanning the aforementioned values. In some embodiments, the one or more additives (collectively or individually) are present in the composition at a dry wt. % of equal to or less than about: 0.001%, 0.01%, 0.1%, 1%, 5%, 7.5%, 10%, 15%, 20%, 25%, 35%, 50%, 60%, 70%, 80%, 90%, 95% or ranges including and/or spanning the aforementioned values. In some embodiments, the one or more additives (collectively or individually) are present in the composition at a wet wt % of equal to or less than about: 0.001%, 0.01%, 0.1%, 0.1%, 0.5%, 0.75%, 1.0%, 1.5%, 2.0%, 2.5%, 5.0%, 10%, 15%, 20%, 30%, 40%, 50%, or ranges including and/or spanning the aforementioned values. In some embodiments, as disclosed elsewhere herein, the composition is aqueous, while in others it has been dried into a powder. In some embodiments, the one or more additives, surprisingly, stabilize the lipid composition when in powdered form (e.g., dry or substantially dry form). In some embodiments, the one or more additives surprisingly help the lipid composition to return to particle form (e.g., nano or microparticle form) when reconstituted.

### E. Phospholipids

As disclosed herein, the lipid-based particle composition comprises one or more phospholipids. In some embodiments, the one or more phospholipids comprises one or more of phosphatidic acid, phosphatidylethanolamine, phosphatidylcholine, phosphatidylserine, phosphatidylinositol, phosphatidylinositol phosphate, phosphatidylinositol bisphosphate, and phosphatidylinositol trisphosphate. In some embodiments, the phospholipid component is primarily phosphatidylcholine. In some embodiments, the only phospholipid present is phosphatidylcholine (e.g., the phospholipid lacks phospholipids other than phosphatidylcholine or is substantially free of other phospholipids). In some embodiments, the one or more phospholipid components (e.g., phosphatidylcholine, and/or others), collectively or individually, are present in the aqueous composition at a concentration of less than or equal to about: 600 mg/ml, 500 mg/ml, 400 mg/ml, 300 mg/ml, 200 mg/ml, 150 mg/ml, 100 mg/ml, 75 mg/ml, 50 mg/ml, 25 mg/ml, 10 mg/ml, or ranges including and/or spanning the aforementioned values. For instance, in some embodiments where two or more phospholipids are present (e.g., phosphatidylcholine, phosphatidylinositol, and phosphatidylethanolamine, etc.), those phospholipids may be present collectively at a concentration of less than or equal to about: 600 mg/ml, 500 mg/ml, 400 mg/ml, 300 mg/ml, 200 mg/ml, 150 mg/ml, 100 mg/ml, 75 mg/ml, 50 mg/ml, 25 mg/ml, 10 mg/ml, or ranges including and/or spanning the aforementioned values. In some embodiments, the one or more phospholipid(s) (collectively or individually) are present in the composition at a dry wt. % of equal to or less than about: 5%, 10%, 15%, 20%, 25%, 30%, 25%, 40%, 45%, 50%, 55%, 60%, 65%, 70%, 75%, 80%, 85%, 90%, 95%, or ranges including and/or spanning the aforementioned values. In some embodiments, the one or more phospholipid(s) (collectively or individually) are present in the composition at a dry wt % of about 45% to about 90%, In some embodiments, the one or more phospholipid(s) (collectively or individually) are present in the composition at a dry wt % of about 53% to about 75%. In some embodiments, the one or more phospholipid(s) (collectively or individually) are present in the composition at a wet wt. % of equal to or greater than about: 1%, 2%, 3%, 4%, 5%, 6%, 7%, 7.5%, 8%, 9%, 10%, 11%, 12%, 12.5%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, or ranges including and/or spanning the aforementioned values. In some embodiments, the one or more phospholipid(s) (collectively or individually) are present in the composition at a wet wt. % of equal to or about 7.5% to about 25%. In some embodiments, the one or more phospholipid(s) (collectively or individually) are present in the composition at a wet wt. % of equal to or about 10% to about 20%. In some embodiments, as disclosed elsewhere herein, the composition is aqueous, while in others it has been dried into a powder. In some embodiments, the phosphatidylcholine is synthetic, derived from sunflower, soy, egg, or mixtures thereof. In some embodiments, the one or more phospholipids (and/or lipids) can be hydrogenated or non-hydrogenated. In some embodiments, a phosphatidylcholine is comprised in a phosphatidylcholine composition (e.g., a lecithin).

In some embodiments, where a phospholipid (e.g., phosphatidylcholine) is used, the phospholipid (e.g., phosphatidylcholine) may be of high purity. For example, in some embodiments, the high purity phosphatidylcholine includes over 96.3% phosphatidylcholine (hydrogenated) or over 99% phosphatidylcholine (hydrogenated) (PC100). In some embodiments, the high purity phosphatidylcholine composition is lecithin that includes over 95% phosphatidylcholine (non-hydrogenated) (e.g., Table 2) (PC90). In some embodiments, the high purity phosphatidylcholine composition is lecithin and includes over 94% phosphatidylcholine (non-hydrogenated) (e.g., Table 3) (PC85). In some embodiments, the high purity phospholipid composition (e.g., phosphatidylcholine composition) has a purity of greater than or equal to about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 96.3%, 97%, 98%, 99%, 100%, or ranges including and/or spanning the aforementioned values, for a specific phospholipid. In some embodiments, the phospholipid composition (e.g., phosphatidylcholine composition) has a total non-phospholipid % impurity content by weight of less than or equal to about: 30%, 25%, 20%, 17.5%, 15%, 12.5%, 10%, 9.5%, 9%, 8.5%, 8%, 7.5%, 7%, 6.5%, 6%, 5.5%, 5%, 4.5%, 4%, 3.5%, 3%, 2.5%, 2%, 1.5%, 1%, 0.5%, 0%, or ranges including and/or spanning the aforementioned values. In some embodiments, the phospholipid composition (e.g., phosphatidylcholine composition) comprises less than or equal to about 8.5%, 8%, 7.5%, 7%, 6.5%, 6%, 5.5%, 5%, 4.5%, 4%, 3.5%, 3%, 2.5%, 2%, 1.5%, 1%, 0.5%, 0%, (or ranges including and/or spanning the aforementioned values) of any one or more of saturated fatty acids, monounsaturated fatty acids, polyunsaturated fatty acids (C 18), arachidonic acid (ARA) (C 20:4), docosahexaenoic acid DHA (C 22:6), phosphatidic acid, phosphatidylethanolamine, and/or lysophosphatidylcholine by weight. In some embodiments, the phosphatidylcholine composition has less than about 1.1% lysophosphatidylcholine and less than about 2.0% triglycerides by weight. In some embodiments, a composition may comprise Lysophosphatidylcholine (1-LPC), 2-Lysophosphatidylcholine (2-LPC), Phosphatidylethanolamine (PE), N-acylphosphatidylethanolamine (APE), Phosphatidylinositol (PI), and/or Phosphatidic acid (PA). In some embodiments, the concentration of 1-LPC, 2-LPC, PE, APE, PI, and/or PA are at or below 1/10^{th} the concentration of phosphatidylcholine.

In some embodiments, a phospholipid composition (e.g., phosphatidylcholine composition) may be of mixed purity. For example, in some embodiments, the phosphatidylcholine composition is lecithin containing is a mixed purity phosphatidylcholine composition, and includes approximately 37% phosphatidylcholine (hydrogenated), approximately 29% Phosphatidylinositol (PI), and approximately 16% Phosphatidylethanolamine (e.g., Table 1) (PC20).

In some embodiments, a composition may comprise a mixture of at least two different phospholipid compositions (e.g., phosphatidylcholine compositions). In some embodiments, a composition may comprise a mixture of a high purity phospholipid composition (e.g., phosphatidylcholine composition) and a mixed purity phospholipid composition (e.g., phosphatidylcholine composition). In some embodiments, a composition may comprise a mixture of PC20 lecithin (e.g., Table 1), and PC90 lecithin (e.g., Table 2) phospholipids. In some embodiments, a composition may comprise a mixture of PC20 lecithin (e.g., Table 1), and PC85 lecithin (e.g., Table 3) phospholipids.

TABLE 1
PC20 lecithin phospholipid content

| | Molecular | | | | |
|---|---|---|---|---|---|
| | weight | | Content | | |
| Phospholipid | (g/Mol) | mMol | (mg) | Weight-% | Mol-% |
| PC | 770.0 | 0.1088 | 83.8090 | 27.69 | 37.24 |
| 1-LPC | 515.0 | 0.0007 | 0.3676 | 0.12 | 0.24 |
| 2-LPC | 515.0 | 0.0072 | 3.7113 | 1.23 | 2.47 |
| PI | 835.0 | 0.0859 | 71.6966 | 23.68 | 29.38 |
| PS-Na | 797.2 | 0.0034 | 2.6828 | 0.89 | 1.15 |
| PE | 725.0 | 0.0479 | 34.7238 | 11.47 | 16.39 |
| LPE | 470.0 | 0.0025 | 1.1822 | 0.39 | 0.86 |
| APE | 990.0 | 0.0038 | 3.7690 | 1.25 | 1.30 |
| PG | 758.0 | 0.0023 | 1.7263 | 0.57 | 0.78 |
| DPG | 682.5 | 0.0048 | 3.2479 | 1.07 | 1.63 |
| PA | 685.0 | 0.0166 | 11.3396 | 3.75 | 5.66 |
| LPA | 430.0 | 0.0011 | 0.4823 | 0.16 | 0.38 |
| Other | 770.0 | 0.0073 | 5.6536 | 1.87 | 2.51 |

[1]

TABLE 2
PC90 lecithin phospholipid content

| | Molecular | | | | |
|---|---|---|---|---|---|
| | weight | | Content | | |
| Phospholipid | (g/Mol) | mMol | (mg) | Weight-% | Mol-% |
| PC | 770.0 | 0.3479 | 267.8678 | 89.09 | 95.57 |
| 1-LPC | 515.0 | 0.0020 | 1.0292 | 0.34 | 0.55 |
| 2-LPC | 515.0 | 0.0115 | 5.9243 | 1.97 | 3.16 |
| PE | 725.0 | 0.0009 | 0.6361 | 0.21 | 0.24 |
| APE | 990.0 | 0.0002 | 0.1930 | 0.06 | 0.05 |
| PA | 685.0 | 0.0006 | 0.4007 | 0.13 | 0.16 |
| Other | 770.0 | 0.0010 | 0.7506 | 0.25 | 0.27 |

TABLE 3
PC85 lecithin phospholipid content

| | Molecular | | | | |
|---|---|---|---|---|---|
| | weight | | Content | | |
| Phospholipid | (g/Mol) | mMol | (mg) | Weight-% | Mol-% |
| PC | 84.73 | 770.0 | 0.3799 | 91.49 | 94.60 |
| 1-LPC | 0.48 | 515.0 | 0.0022 | 0.35 | 0.54 |
| 2-LPC | 2.94 | 515.0 | 0.0132 | 2.12 | 3.28 |
| PE | 0.45 | 725.0 | 0.0020 | 0.46 | 0.50 |
| APE | 0.20 | 990.0 | 0.0009 | 0.28 | 0.22 |
| PA | 0.49 | 685.0 | 0.0022 | 0.47 | 0.55 |
| Other | 0.28 | 770.0 | 0.0013 | 0.30 | 0.31 |

In certain embodiments, compositions disclosed herein do not comprise a sterol. In certain embodiments, compositions disclosed herein do not comprise detectable levels of a sterol. In certain embodiments, compositions disclosed herein do not comprise sterols at greater than 0.1 w/w %. In certain embodiments, compositions disclosed herein do not comprise triglycerides. In certain embodiments, compositions disclosed herein do not comprise detectable levels of a triglyceride. In certain embodiments, compositions disclosed herein do not comprise a triglyceride at greater than 0.1 w/w %. In certain embodiments, compositions disclosed herein do not comprise an oil. In certain embodiments, compositions disclosed herein do not comprise detectable levels of an oil. In certain embodiments, compositions disclosed herein do not comprise an oil at greater than 0.1 w/w %. In certain embodiments, compositions disclosed herein do not comprise water. In certain embodiments, compositions disclosed herein do not comprise detectable levels of water. In certain embodiments, compositions disclosed herein do not comprise water at greater than 5 w/w %.

In some embodiments, a lipid-based particle composition comprises one or more sterols. In some embodiments, the one or more sterols comprises one or more cholesterols, ergosterols, hopanoids, hydroxysteroids, phytosterols (e.g., vegapure), ecdysteroids, and/or steroids. In some embodiments, the sterol comprises cholesterol. In some embodiments, the sterol is cholesterol. In some embodiments, the only sterol present is cholesterol (e.g., the sterol lacks or substantially lacks sterols other than cholesterol). In some embodiments, the one or more sterol(s) (e.g., cholesterol, and/or other sterols), collectively or individually, are present in the aqueous composition at a concentration of less than or equal to about: 100 mg/ml, 50 mg/ml, 40 mg/ml, 20 mg/ml, 10 mg/ml, 5 mg/ml, 1 mg/ml, 0.1 mg/ml, or ranges including and/or spanning the aforementioned values. In some embodiments, the one or more sterol(s) are present in the composition at a dry wt. % of equal to or less than about: 0.001%, 0.01%, 0.05%, 0.1%, 0.15%, 0.2%, 0.25%, 0.5%, 1%, 5%, 7.5%, 10%, 15%, 20%, 25%, 30%, 40%, or ranges including and/or spanning the aforementioned values. In some embodiments, the one or more sterol(s) (collectively or individually) are present in the composition at a wet wt. % of equal to or less than about: 0.001%, 0.01%, 0.05%, 0.1%, 0.15%, 0.2%, 0.25%, 0.5%, 1%, 2%, 3%, 4%, 5%, 7.5%, 10%, 15%, 20%, or ranges including and/or spanning the aforementioned values. In some embodiments, as disclosed elsewhere herein, the composition is aqueous, while in others it has been dried into a powder. In some embodiments, the cholesterol used in the composition comprises cholesterol from one or more of sheep's wool, synthetic cholesterol, or semisynthetic cholesterol from plant origin. In some embodiments, the sterol (or combination of sterols) has a purity of greater than or equal to about: 92.5%, 95%, 96%, 97%, 98%, 99%, 99.5%, 99.9%, 100.0%, or ranges including and/or spanning the aforementioned values. In some embodiments, the sterol has a total % impurity content by weight of less than or equal to about: 8.5%, 8%, 7.5%, 7%, 6.5%, 6%, 5.5%, 5%, 4.5%, 4%, 3.5%, 3%, 2.5%, 2%, 1.5%, 1%, 0.5%, 0%, or ranges including and/or spanning the aforementioned values. In some embodiments, the sterol is cholesterol. In some embodiments, the sterol is not cholesterol. In some embodiments, the sterol is phytosterol.

### F. Preservatives

In some embodiments, the lipid-based particle composition comprises a preservative. In some embodiments, the preservative includes one or more benzoates (such as sodium benzoate or potassium benzoate), nitrites (such as sodium nitrite), sulfites (such as sulfur dioxide, sodium or potassium sulphite, bisulphite or metabisulphite), sorbates (such as sodium sorbate, potassium sorbate), ethylenediaminetetraacetic acid (EDTA) (and/or the disodium salt thereof), polyphosphates, organic acids (e.g., citric, succinic, malic, tartaric, benzoic, lactic and propionic acids), and/or antioxidants (e.g., vitamins such as vitamin E and/or vitamin C, butylated hydroxytoluene). In some embodiments, the one or more preservatives, collectively or individually, are present in the aqueous composition at a concentration of less than or equal to about: 10 mg/ml, 5 mg/ml, 1 mg/ml, 0.85 mg/ml, 0.5 mg/ml, 0.1 mg/ml, or ranges including and/or spanning the aforementioned values. In some embodiments, the one or more preservatives (collectively or individually) are present in the composition at a dry wt % of equal to or at less than about: 0.01%, 0.1%, 0.25%, 0.5%, 1%, 5%, 7.5%, 10%, 15%, 20%, 25%, or ranges including and/or spanning the aforementioned values. In some embodiments, the one or more preservatives (collectively or individually) are present in the composition at a wet wt. % of equal to or less than about: 0.001%, 0.01%, 0.025%, 0.05%, 0.1%, 0.5%, 0.75%, 1.0%, 1.5%, 2.0%, 2.5%, 5%, or ranges including and/or spanning the aforementioned values. In some embodiments, as disclosed elsewhere herein, the composition is aqueous, while in others it has been dried into a powder. In some embodiments, the aqueous composition comprises one or more of malic acid at about 0.85 mg/ml, citric acid at about 0.85 mg/ml, potassium sorbate at about 1 mg/ml, and sodium benzoate at about 1 mg/ml. In some embodiments, the preservatives inhibit or prevent growth of mold, bacteria, and fungus. In some embodiments, Vitamin E is added at 0.5 mg/ml to act as an antioxidant in the oil phase. In some embodiments, the preservative concentrations may be changed depending on the flavored oil used. In some embodiments, the preservative concentrations may be changed depending on the therapeutic ingredients used.

### G. Flavoring Agents and Dyes

In some embodiments, the lipid-based particle composition comprises one or more flavoring agents. In some instances, the one or more flavoring agents is an active agent. In some embodiments, the one or more flavoring agent(s), collectively or individually, are present in the aqueous composition at a concentration of less than or equal to about: 10 mg/ml, 5 mg/ml, 1.5 mg/ml, 1.2 mg/ml, 1 mg/ml, 0.9 mg/ml, 0.5 mg/ml, 0.1 mg/ml, or ranges including and/or spanning the aforementioned values. In some embodiments, the one or more flavoring agent(s) (collectively or individually) are present in the composition at a dry wt. % of equal to or less than about: 0.01%, 0.1%, 0.25%, 0.5%, 1%, 5%, 7.5%, 10%, 15%, 20%, 25%, or ranges including and/or spanning the aforementioned values. In some embodiments, the one or more flavoring agents (collectively or individually) are present in the composition at a wet wt. % of equal to or less than about: 0.001%, 0.01%, 0.025%, 0.05%, 0.1%, 0.5%, 0.75%, 1.0%, 1.5%, 2.0%, 2.5%, 5.0%, 10%, or ranges including and/or spanning the aforementioned values. In some embodiments, as disclosed elsewhere herein, the composition is aqueous, while in others it has been dried into a powder. In some embodiments, the one or more flavoring agents of the composition comprise monk fruit extract, steviol glycosides, peppermint oil, lemon oil, vanilla, or the like, or combinations thereof. In some embodiments, the composition contains monk fruit extract at 0.9 mg/ml and flavored oils as flavoring. Examples of flavored oils are peppermint and lemon at 1.2 mg/ml. Chemicals that are not oil may also be used for flavor, for example, such as dry powders that replicate a flavor such as vanilla. In some embodiments, a flavoring agent is and/or comprises maltodextrin.

In some embodiments, the lipid-based particle composition comprises one or more dyes (e.g., colorants). In some instances, the one or more dyes is an active agent. In some instances, the one or more dyes is not synthetic. In some embodiments, the one or more dye(s), collectively or individually, are present in the aqueous composition at a concentration of less than or equal to about: 10 mg/ml, 5 mg/ml, 1.5 mg/ml, 1.2 mg/ml, 1 mg/ml, 0.9 mg/ml, 0.5 mg/ml, 0.1 mg/ml, or ranges including and/or spanning the aforementioned values. In some embodiments, the one or more dye(s) (collectively or individually) are present in the composition at a dry wt. % of equal to or less than about: 0.01%, 0.1%, 0.25%, 0.5%, 1%, 5%, 7.5%, 10%, 15%, 20%, 25%, or ranges including and/or spanning the aforementioned values. In some embodiments, the one or more dye(s) (collectively or individually) are present in the composition at a wet wt. % of equal to or less than about: 0.001%, 0.01%, 0.025%, 0.05%, 0.1%, 0.5%, 0.75%, 1.0%, 1.5%, 2.0%, 2.5%, 5.0%, 10%, or ranges including and/or spanning the aforementioned values. In some embodiments, as disclosed elsewhere herein, the composition is aqueous, while in others it has been dried into a powder.

In some instances, the dyes are not synthetic dyes. In some instances, the dyes are natural dyes. In some instances, the natural dyes include anthocyanins. In some instances, the dyes are more stable in the nanoparticles disclosed herein than in a similar nanoparticle that does not contain the plant extract. In some instances, the natural dye is a purple sweet potato extract, black carrot extract, red radish extract, red cabbage extract, grape extract, grape skin extract, aronia extract, elderberry extract, and/or hibiscus extract.

### H. Water Content

In some embodiments, the lipid-based particle composition is aqueous while in other embodiments the composition may be provided as a dry or substantially dry solid (e.g., having a water content in weight % of less than or equal to 25%, 20%, 15%, 10%, 5%, 2%, 1%, 0.5%, 0.1%, 0%, or ranges including and/or spanning the aforementioned values). In some embodiments, where the lipid-based particle composition is aqueous, water may be present at a wet weight percent of equal to or less than about: 20%, 25%, 30%, 40%, 50%, 60%, 70%, 75%, 77%, 80%, 85%, 90%, 95%, 97.5%, 99%, or ranges including and/or spanning the aforementioned values. In some instances where the composition is dry or substantially dry, water may be present at a wt. % of at, less than, between, or a range of 10%, 9%, 8%, 7%, 6%, 5%, 4%, 3%, 2%, 1%, or 0%. In some instances, the composition is a dry powder. In some instances, the nanoparticles are a dry powder.

In several embodiments, as disclosed elsewhere herein, the composition is aqueous, while in others it has been dried into a powder (that is free of or substantially free of water). In several embodiments, where the composition has been dried, it comprises a water content of less than or equal to 20%, 15%, 10%, 7.5%, 5%, 2.5%, 1%, or ranges including and/or spanning the aforementioned values.

In several embodiments, as disclosed elsewhere herein, the composition is aqueous (e.g., contains water) while in other embodiments, the composition is dry (lacks water or substantially lacks water). In several embodiments, the composition has been dried (e.g., has been subjected to a process to remove most or substantially all water). In several embodiments, the composition comprises nanoparticles in water (e.g., as a solution, suspension, or emulsion). In other embodiments, the composition is provided as a powder (e.g., that may be constituted or reconstituted in water). In several embodiments, as disclosed elsewhere herein, the water content (in wt. %) of the composition is less than or equal to about: 30%, 20%, 10%, 5%, 2.5%, 1%, 0.5%, 0.1%, 0%, or ranges including and/or spanning the aforementioned values. In several embodiments, as disclosed elsewhere herein, the water content (in wt. %) of the composition is greater than or equal to about: 50%, 60%, 70%, 80%, 85%, 90%, 92.5%, 95%, 97.5%, or ranges including and/or spanning the aforementioned values. In several embodiments, the water is nanopure, deionized, USP grade, WFI, drinkable municipal water, and/or combinations of the foregoing. In some aspects, the composition is a dried composition comprising a nanoparticle having weight ratios of a first therapeutic active agent: a lipid source: a saponin; and optionally a surfactant of 0.0015 to 50 of a first therapeutic active agent:2 to 80 of a lipid source:0.0015 to 10 of a plant extract:0 to 17.5 of a surfactant.

### I. Bulking Agents and Other Additives

In some embodiments, the lipid-based particle composition comprises one or more bulking agents (e.g., carbohydrates, polymers, etc.). In some embodiments, the lipid-based particle composition comprises one or more carbohydrates (and/or a carbohydrate source). In some embodiments, a bulking agent comprises a polymer. In certain embodiments, a bulking agent comprises acacia/Arabic gum. In some embodiments, a bulking agent comprises one or more types of fiber derived from one or more different sources. In some embodiments, the bulking agent is selected from the group consisting of a saccharide, an oligosaccharide, a polysaccharide, a monosaccharide, a protein, a lipid, trehalose, sucrose, dextrose, glucose, isomaltulose, tagatose, arabinose, maltose, fructose, dextrin, lactose, maltose, fucose, galactose, a gum, inositol, maltodextrin, maltol, mannose, muscovado, ribose, rhamnose, saccharose, sucralose, xylose, lecithin, fiber, avocado fiber, acacia fiber, psyllium fiber, beta-glucan, guar gum, xanthan gum, pectin, chitin, cellulose, a modified cellulose, methylcellulose, hemicellulose, microcrystalline cellulose, beta cyclodextrin, an alginate salt, sodium alginate, potassium alginate, ammonium alginate, calcium alginate, ammonium alginate, propylene glycol alginate, agar, carrageen, raffinose, polydextrose, cyclodextrins, fullerene, inulin, gelatin, pentose, and combinations and/or polymers thereof. In some embodiments, one or more bulking agents comprises polydextrose, inulin, FOS, resistant starch, and/or maltodextrins. In some embodiments, the one or more bulking agents, collectively or individually, are present in the aqueous composition at a concentration of less than or equal to about: 400 mg/mL, 350 mg/mL, 300 mg/mL, 250 mg/mL, 200 mg/mL, 175 mg/mL, 150 mg/mL, 125 mg/mL, 100 mg/ml, 90 mg/ml, 80 mg/ml, 70 mg/ml, 60 mg/ml, 50 mg/ml, 40 mg/ml, 30 mg/ml, 20 mg/ml, 10 mg/ml, 5 mg/ml, 1.5 mg/ml, 1.2 mg/ml, 1 mg/ml, 0.9 mg/ml, 0.5 mg/ml, 0.1 mg/ml, 0.01 mg/ml, 0.001 mg/ml or ranges including and/or spanning the aforementioned values. In some embodiments, the one or more bulking agents (collectively or individually) are present in the composition at a dry wt. % of equal to or less than about: 0.001%, 0.01%, 0.1%, 1%, 5%, 7.5%, 10%, 15%, 20%, 25%, 35%, 50%, 60%, 70%, 80%, 90%, 95% or ranges including and/or spanning the aforementioned values. In some embodiments, the one or more bulking agents (collectively or individually) are present in the composition at a wet wt. % of equal to or less than about: 0.001%, 0.01%, 0.1%, 0.1%, 0.5%, 0.75%, 1.0%, 1.5%, 2.0%, 2.5%, 5.0%, 10%, 15%, 20%, 30%, 40%, 50%, or ranges including and/or spanning the aforementioned values. In some embodiments, as disclosed elsewhere herein, the composition is aqueous, while in others it has been dried into a powder. In some embodiments, the one or more bulking agents, surprisingly, stabilize the lipid composition when in powdered form (e.g., dry or substantially dry form). In some embodiments, the one or more bulking agents surprisingly help the lipid composition to return to particle form (e.g., nano or microparticle form) when reconstituted.

In several embodiments, the nanoparticle (or a composition comprising the nanoparticle) is composed and/or comprises one or more extracts from the same plant species. In several embodiments, the one or more extracts may be from any one or more plant strains.

In several embodiments, the nanoparticle (or compositions comprising the nanoparticle) comprises or is composed of plant powders and/or plant active ingredients (e.g., including but not limited to alkaloids). In other embodiments, the extracts may be produced synthetically (e.g., in a laboratory). In several embodiments, the synthetic extract may share a structure with an extract that is naturally occurring. In several embodiments, the extracts are analogs of natural extracts of a plant (e.g., produced synthetically).

In several embodiments, the plant extracts are isolated from their plant sources. In several embodiments, the plant extracts are isolated from their plant sources using solvent extraction. In several embodiments, the plant extracts are isolated from their plant sources using acid base titration. In several embodiments, the plant extracts are isolated from their plant sources using CO₂ (supercritical or nonsupercritical). In several embodiments, the plant extracts are isolated from their plant sources using cryogenic ethanol. In several embodiments, the plant extracts are isolated from their plant sources using other forms of extraction. In several embodiments, the extract is an alkaloid, as disclosed elsewhere herein.

In several embodiments, the nanoparticle composition comprises or excludes a preservative. In several embodiments, the preservative includes or excludes one or more benzoates (such as sodium benzoate or potassium benzoate), nitrites (such as sodium nitrite), sulfites (such as sulfur dioxide, sodium or potassium sulphite, bisulphite or metabisulphite), sorbates (such as sodium sorbate, potassium sorbate), ethylenediaminetetraacetic acid (EDTA) (and/or the disodium salt thereof), polyphosphates, plant oils, citrus oils, polyphenols, synthetic polymers, natural polymers, fermented cultures, organic acids (e.g., citric, succinic, ascorbic, malic, tartaric, benzoic, lactic and propionic acids), extracts (e.g., mushroom extract) and/or antioxidants (e.g., vitamins such as vitamin E and/or vitamin C, butylated hydroxytoluene). In several embodiments, sorbates and benzoates may be used in acidic pH formulations. In several embodiments, the one or more preservatives (collectively or individually) are present in the composition at a dry wt. % of equal to or at less than about: 0%, 0.01%, 0.1%, 0.25%, 0.5%, 1%, 5%, 7.5%, 10%, 15%, 20%, 25%, or ranges including and/or spanning the aforementioned values. In several embodiments, the one or more preservatives (collectively or individually) are present in the composition at a wet wt. % of equal to or less than about: 0%, 0.001%, 0.01%, 0.025%, 0.05%, 0.1%, 0.5%, 0.75%, 1.0%, 1.5%, 2.0%, 2.5%, 5%, or ranges including and/or spanning the aforementioned values. In several embodiments, the one or more surfactants (collectively or individually) are present in the composition at a wet w/v of equal to or less than about: 0 mg/mL, 0.001 mg/mL, 0.1 mg/mL, 0.5 mg/mL, 1.0 mg/mL, 2.5 mg/mL, 4 mg/mL, 5 mg/mL, or ranges including and/or spanning the aforementioned values. In several embodiments, as disclosed elsewhere herein, the composition is aqueous, while in others it has been dried into a powder (dry). In several embodiments, the preservatives inhibit or prevent growth of mold, bacteria, and fungus.

In several embodiments, the nanoparticle composition comprises or excludes one or more flavoring agents. In some instances, the one or more flavoring agents is an active agent. In several embodiments, the one or more flavoring agent(s) comprise or exclude an essential oil (or combinations of essential oils). In several embodiments, the one or more flavoring agents of the composition comprise or exclude monk fruit extract, steviol glycosides, glycerin, peppermint oil or flavoring, lemon oil or flavoring, orange oil or flavoring, vanilla, taste makers, bitter blockers, or the like, or combinations thereof. In several embodiments, the one or more flavoring agent(s) (collectively or individually) are present in the composition at a dry wt. % of equal to or less than about: 0%, 0.01%, 0.1%, 0.25%, 0.5%, 1%, 5%, 7.5%, 10%, 15%, 20%, 25%, or ranges including and/or spanning the aforementioned values. In several embodiments, the one or more flavoring agents (collectively or individually) are present in the composition at a wet wt. % of equal to or less than about: 0%, 0.001%, 0.01%, 0.025%, 0.05%, 0.1%, 0.5%, 0.75%, 1.0%, 1.5%, 2.0%, 2.5%, 5.0%, or ranges including and/or spanning the aforementioned values. In several embodiments, as disclosed elsewhere herein, the composition is aqueous, while in others it has been dried into a powder. For instance, as disclosed elsewhere herein, in several embodiments, the composition is aqueous (wet), while in others it has been dried into a powder (dry).

In some embodiments, the nanoparticle composition comprises one or more dyes (e.g., colorants). In some instances, the one or more dyes is an active agent. In some embodiments, the one or more dye(s), collectively or individually, are present in the aqueous composition at a concentration of less than or equal to about: 10 mg/ml, 5 mg/ml, 1.5 mg/ml, 1.2 mg/ml, 1 mg/ml, 0.9 mg/ml, 0.5 mg/ml, 0.1 mg/ml, or ranges including and/or spanning the aforementioned values. In some embodiments, the one or more dye(s) (collectively or individually) are present in the composition at a dry wt. % of equal to or less than about: 0.01%, 0.1%, 0.25%, 0.5%, 1%, 5%, 7.5%, 10%, 15%, 20%, 25%, or ranges including and/or spanning the aforementioned values. In some embodiments, the one or more dye(s) (collectively or individually) are present in the composition at a wet wt. % of equal to or less than about: 0.001%, 0.01%, 0.025%, 0.05%, 0.1%, 0.5%, 0.75%, 1.0%, 1.5%, 2.0%, 2.5%, 5.0%, 10%, or ranges including and/or spanning the aforementioned values. In some embodiments, as disclosed elsewhere herein, the composition is aqueous, while in others it has been dried into a powder.

In some instances, the dyes are not synthetic dyes. In some instances, the dyes are natural dyes. In some instances, the natural dyes include anthocyanins. In some instances, the dyes are more stable in the nanoparticles disclosed herein than in a similar nanoparticle that does not contain the plant extract. In some instances, the natural dye is a purple sweet potato extract, black carrot extract, red radish extract, red cabbage extract, grape extract, grape skin extract, aronia extract, elderberry extract, and/or hibiscus extract.

In several embodiments, the composition comprises, consists of, or consists essentially of one or more of the ingredients, compounds, components, etc. disclosed herein.

In several embodiments, as disclosed elsewhere herein, the nanoparticle composition provides particles in the nano-measurement range. In several embodiments, the nanoparticle is spherical or substantially spherical. In several embodiments, a solid lipid nanoparticle possesses a solid lipid core matrix that can solubilize lipophilic molecules. In several embodiments, the lipid core is stabilized by surfactants and/or emulsifiers as disclosed elsewhere herein, while in other embodiments, surfactants are absent. In several embodiments, the size of the particle is measured as a mean diameter. In several embodiments, the size of the particle is measured by dynamic light scattering. In several embodiments, the size of the particle is measured using a zeta-sizer. In several embodiments, the size of the particle can be measured using Scanning Electron Microscopy (SEM). In several embodiments, the size of the particle is measured using a cryogenic SEM (cryo-SEM). Where the size of a nanoparticle is disclosed elsewhere herein, any one or more of these instruments or methods may be used to measure such sizes.

In several embodiments, the nanoparticle composition comprises nanoparticles having an average size of less than, greater than, between, or equal to about: 10 nm, 25 nm, 40 nm, 50 nm, 100 nm, 250 nm, 500 nm, 1000 nm, or ranges including and/or spanning the aforementioned values. In several embodiments, the composition comprises nanoparticles having an average size of between about 50 nm and 150 nm or between about 50 and about 250 nm. In several embodiments, the size distribution of the nanoparticles for at least 50%, 75%, 80%, 90% (or ranges including and/or spanning the aforementioned percentages) of the particles present is equal to or less than about: 20 nm, 40 nm, 60 nm, 80 nm, 100 nm, 110 nm, 120 nm, 130 nm, 140 nm, 160 nm, 180 nm, 200 nm, 300 nm, 400 nm, 500 nm, or ranges including and/or spanning the aforementioned nm values. In several embodiments, the size distribution of the nanoparticles for at least 90% of the particles present is equal to, between, greater than, or less than about: 20 nm, 40 nm, 60 nm, 80 nm, 100 nm, 110 nm, 120 nm, 130 nm, 140 nm, 160 nm, 180 nm, 200 nm, 300 nm, 400 nm, 500 nm, or ranges including and/or spanning the aforementioned nm values. In several embodiments, the D90 of the particles present is equal to, between, greater than, or less than about: 80 nm, 100 nm, 110 nm, 120 nm, 130 nm, 140 nm, 160 nm, 180 nm, 200 nm, 300 nm, 400 nm, 500 nm, or ranges including and/or spanning the aforementioned values. In several embodiments, the size of the nanoparticle is the diameter of the nanoparticle as measured using any of the techniques as disclosed elsewhere herein. For instance, in some embodiments, the size of the nanoparticle is the measured using dynamic light scattering. In several embodiments, the size of the nanoparticle is the measured using a zeta sizer. In several embodiments, consistency in size over time, or within a sample, allows predictable stability for the active agent encapsulated therein.

In several embodiments, over 50%, 75%, 95% (or ranges spanning and or including the aforementioned values) of the nanoparticles prepared by the methods disclosed herein have a particle size of between about 20 to about 500 nm (as measured by zeta sizing (e.g., refractive index). In several embodiments, over 50%, 75%, 95% (or ranges spanning and or including the aforementioned values) of the nanoparticles prepared by the methods disclosed herein have a particle size of between about 50 nm to about 200 nm (as measured by zeta sizing (e.g., refractive index). In several embodiments, over 50%, 75%, 95% (or ranges spanning and or including the aforementioned values) of the nanoparticles prepared by the methods disclosed herein have a particle size of between about 90 nm to about 150 nm (as measured by zeta sizing (e.g., refractive index). In several embodiments, this consistency in size allows predictable delivery to subjects. In several embodiments, the D90 particle size measurement varies between 150 and 500 nm.

In several embodiments, as disclosed elsewhere herein, the nanoparticle composition is an oil-in-water emulsion, water-in-oil emulsion, water-in-oil-in-water emulsion, oil-in-water-in-oil emulsion, liposome, solid lipid particles formulation, etc. For brevity, these may just be referred to as the composition. In several embodiments, the nanoparticle composition can be processed to comprises one or more of solid lipid nanoparticles, liposomes (and variants including multi-lamellar, double liposome preparations, etc.), niosomes, ethosomes, electrostatic particulates, microemulsions, nanoemulsions, microsuspensions, nanosuspensions, or combinations thereof. In several embodiments, polymeric nanoparticles may be formed. In several embodiments, cyclodextrin is added or is excluded.

In several embodiments, as disclosed elsewhere herein, the nanoparticle composition is a dry or substantially dry composition. In some instances, the dry or substantially dry composition is a powder, a pellet, a plurality of particles, a film, a pill, a tablet, etc. In some instances, the dry or substantially dry composition is an oily liquid.

In some instances, composition comprises a carrier, such as a pharmaceutically acceptable carrier. The carrier can be, but is not limited to distilled water, physiological phosphate-buffered saline, normal or lactated Ringer's solutions, dextrose solution, Hank's solution, propanediol, a carbohydrate. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve. The carrier can be a solid carrier, such as a carbohydrate, clay, a salt, an inert powder, a polymer, chalk, etc.

In several embodiments, solid lipid nanoparticle compositions comprises a lipid core matrix. In several embodiments, the lipid core matrix is solid. In several embodiments, the solid lipid comprises one or more ingredients as disclosed elsewhere herein. In several embodiments, the core of the solid lipid comprises one or more lipids, surfactants, active ingredients, etc. In several embodiments, the surfactant acts as an emulsifier. In several embodiments, emulsifiers can be used to stabilize the lipid dispersion (with respect to charge and molecular weight). In several embodiments, the core ingredients (e.g., the components of the core) are present in the composition (collectively or individually) at a dry wt. % of equal to or less than about: 0.5%, 1.0%, 2.5%, 5%, 10%, 15%, 20%, 30%, 40%, 50%, 60%, 80% or ranges including and/or spanning the aforementioned values. In several embodiments, the core ingredients and/or the emulsifiers (collectively or individually) are present in the composition at a wet wt. % of equal to or less than about: 0.5%, 1.0% 2.5%, 5%, 7.5%, 10%, 12.5%, 15%, 20%, 30%, 40%, 60% or ranges including and/or spanning the aforementioned values.

In several embodiments, the nanoparticle composition (e.g., when in water or dried) comprises multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, and/or combinations thereof. In several embodiments, the composition is characterized by having multiple types of particles (e.g., lamellar, emulsion, irregular, etc.). In other embodiments, a majority of the particles present are emulsion particles. In several embodiments, a majority of the particles present are lamellar (multilamellar and/or unilamellar). In other embodiments, a majority of the particles present are irregular particles. In still other embodiments, a minority of the particles present are emulsion particles. In several embodiments, a minority of the particles present are lamellar (multilamellar and/or unilamellar). In other embodiments, a minority of the particles present are irregular particles.

In several embodiments, at ambient temperature an aqueous nanoparticle composition as disclosed herein has a viscosity (in centipoise (cP)) at about 25° C. or 26° C. of equal to or less than about: 1.0, 1.05, 1.1, 1.2, 1.5, 2.0, 5.0, 10.0, 20, 30, 50, 100, 200, 300, 500, 1000, 2000, 3000, 5000, or ranges including and/or spanning the aforementioned values. In several embodiments, at about 25° C. or 26° C. and a concentration of 20 mg/mL active agent in water (e.g., the total nanoparticle composition may have a concentration of 50 to 250 mg/mL), the nanoparticle composition has a viscosity (in centipoise (cP)) of equal to or less than about: 1.0, 1.05, 1.1, 1.2, 1.5, 2.0, 5.0, 10.0, 20, 30, 50, 100, 200, 300, 500, 1000, 2000, 3000, 5000, or ranges including and/or spanning the aforementioned values. In several embodiments, at about 25° C. and a concentration of 50 mg/mL, 100 mg/mL, 200 mg/mL, or 250 mg/mL, the nanoparticle composition has a viscosity (in cP) of equal to or less than about: 1.0, 1.05, 1.1, 1.2, 1.5, 2.0, 5.0, 10.0, 20, 30, 50, 100, 200, 300, 500, 1000, 2000, 3000, 5000, or ranges including and/or spanning the aforementioned values. In several embodiments, the viscosity of the lipid nanoparticle aqueous solution is equal to or less than 5.0 Cp.

### J. Additional Components, Additives, or Methods

In some embodiments, the lipid-based particle composition (e.g., when in water or dried) comprises multilamellar nanoparticle vesicles, unilamellar nanoparticle vesicles, multivesicular nanoparticles, and/or emulsion particles. In some embodiments, the composition is characterized by having multiple types of particles (e.g., multilamellar, unilamellar, and emulsion, etc.). In some embodiments, a majority of the particles present are lamellar (multilamellar and/or unilamellar) particles. In some embodiments, a majority of the particles present are unilamellar particles. In some embodiments, a minority of the particles present are emulsion particles.

In some embodiments, at ambient temperature an aqueous lipid-based composition as disclosed herein has a viscosity (in centipoise (cP)) of equal to or less than about: 1.0, 1.05, 1.1, 1.2, 1.5, 2.0, 5.0, 10.0, 20, 30, 50, 100, 150, 200, 250, 300, 350, 400, 450, 500, 600, 700, 800, 900, 1000, 2000, 3000, 4000, 5000, or ranges including and/or spanning the aforementioned values.

In some embodiments, the liposomes and/or a liquid (e.g., aqueous) composition comprising the nanoparticles as disclosed herein are lyophilized. In some embodiments, where lyophilization is used to prepare a liposomal- and/or nanoparticle-based powder, one or more lyoprotectant agents may be added. In some embodiments, an individual lyoprotectant agent may be present at a dry wt. % equal to or less than the dry weight of the lipophilic ingredients. In some embodiments, the lyoprotectant agent(s) (collectively or individually) may be present at a dry wt. % equal to or less than about: 5%, 10%, 15%, 20%, 30%, 40%, 50%, 60%, or ranges including and/or spanning the aforementioned values. In some embodiments, the lyoprotectant agent(s) (collectively or individually) may be present at a wet wt % of equal to or less than about: 2.5%, 5%, 7.5%, 10%, 12.5%, 15%, 20%, 30%, or ranges including and/or spanning the aforementioned values. In some embodiments, the lyoprotectant is selected from the group consisting of lactose, dextrose, trehalose, arginine, glycine, histidine, and/or combinations thereof. In several embodiments, the nanoparticle compositions herein are spray dried (e.g., to provide a powder). In several embodiments, the nanoparticle compositions are spray dried and not lyophilized. In some embodiments, the nanoparticle composition is spray dried, fluid bed dried, desiccated, and/or lyophilized.

Nanoparticle formulations containing active agents can be formed into spray dried powders. Formulations containing active agents alone with varying amounts of various lipids and other excipients can be prepared using a solvent-free manufacturing process. Water-soluble components can be dissolved in water at 65° C. with magnetic stirring. High shear mixing can be applied at 65° C. and lipids and excipients can be added. High shear mixing can be maintained until a stable suspension is formed. The suspension can then optionally be microfluidized for 1, 2, 3, 4, or 5 passes using an MP110 microfluidizer at 5,000, 10,000, 15,000, 20,000, 25,000, or 30,000 psi. Following microfluidization, formulations can be diluted with an equal volume containing the excipient type such that the excipient is at the stated final concentration. Up to 25% ethanol (e.g., 10%, 15%, 20%, 25%) can be added to the formulation and mixed to a homogenous solution prior to spray drying. Formulations can be spray dried on a Buchi B290 benchtop spray dryer. The inlet temperature can be set at 125° C., the aspirator set to 100%, the pump rate set to 10%, and nitrogen flow set to 60 mmHg. Powder can be collected and measured for residual moisture, and recovered yield can be calculated.

As disclosed elsewhere herein, some embodiments pertain to methods of preparing lipid-based particle compositions comprising nanoparticles and/or liposomes. In some embodiments, the composition is prepared by forming a lipid-in-oil emulsion. In some embodiments, an oil-in-water emulsion can be prepared without the use of organic solvents. In some embodiments, a composition can be prepared without the use of organic solvents. In some embodiments, a composition can be prepared in a solvent-free process. In some embodiments, solid ingredients are added and dissolved into liquid ingredients In some embodiments, the phospholipid composition (e.g., phosphatidylcholine composition) can be added with mixing. In some embodiments, when a well dispersed lipid phase is formed after mixing, the addition of water (e.g., having a temperature of equal to or at least about: 0.1° C., 1° C., 5° C., 10° C., 20° C., 30° C., 40° C., 50° C., 60° C., 80° C., or ranges including and/or spanning the aforementioned values) and additional mixing achieves an oil-in-water emulsion. In some embodiments, the oil-in-water emulsion is then subject to high-shear mixing to form nanoparticles. In some embodiments, high-shear mixing is performed using a high shear dispersion unit or an in-line mixer can be used to prepare the emulsions. In some embodiments, the particles can be made by solvent evaporation and/or solvent precipitation. In some embodiments, the particles can be made in a solvent-free process. In some embodiments, the particles can be made in solvent-free process that does not comprise solvent evaporation and/or solvent precipitation.

In some embodiments, oil-in-water emulsion is subject to high-pressure homogenization using a microfluidizer. In some embodiments, high sheer mixing can be used to reduce the particle size. In some embodiments, the oil-in-water emulsion is processed to a nanoparticle (e.g., about 20 to about 500 nm, etc.) using the microfluidizer or other high sheer processes. In some embodiments, the oil-in-water emulsion is processed to a nanoparticle having a size from about 30 nm to about 200 nm in diameter, about 80 nm to about 180 nm in diameter, about 50 nm to about 150 nm in diameter, or about 100 nm to about 150 nm in diameter.

In some embodiments, an oil-in-water emulsion is passed through the microfluidizer a plurality of times (e.g., equal to or at least 1 time, 2 times, 3 times, 4 times, 5 times, 10 times, or ranges including and/or spanning the aforementioned values). In some embodiments, an oil-in-water emulsion is passed through the microfluidizer in discreet volumes per pass. In some embodiments, an oil-in-water emulsion is passed through the microfluidizer in non-discreet volumes in a continuous process. In some embodiments, an emulsion is passed through the microfluidizer at a pressure of equal to or less than about: 5,000 PSI, 15,000 PSI, 20,000 PSI, 25,000 PSI, 30,000 PSI, or ranges including and/or spanning the aforementioned values. In some embodiments, the emulsion is passed through the microfluidizer at a temperature of equal to or at least about: 30° C., 40° C., 50° C., 65° C., 80° C., or ranges including and/or spanning the aforementioned values. In some embodiments, the emulsion is passed through the microfluidizer at least about room temperature (e.g., about 20° C. or about 25° C.) and/or without any heating and/or temperature control. In some embodiments, the emulsion is passed through the microfluidizer at a temperature of equal to or less than about 80° C. In some embodiments, the microfluidizer includes an interaction chamber consisting of 75 μm to 200 μm pore sizes and the emulsion is passed through this chamber. In some embodiments, the pore size of the microfluidizer are less than or equal to about: 75 μm, 100 μm, 150 μm, 200 μm, 250 μm, 300 μm, or ranges including and/or spanning the aforementioned values. In some embodiments, the nanoparticle composition is prepared by high shear mixing, sonication, and/or extrusion.

In some embodiments, after preparation, the lipid-based particle composition is characterized by an ability to pass through a 0.2 μm filter while preserving the nanoparticle structure (e.g., a change in average nanoparticle size of no greater than 10 nm, 20 nm, or 30 nm). In some embodiments, after passage through a 0.2 μm there is a change in average diameter of the particles of equal to or at less than about: 1%, 5%, 10%, 20%, or ranges including and/or spanning the aforementioned values. In some embodiments, after passage through a 0.2 μm there is a change in PDI of the particles of equal to or at less than about: 1%, 5%, 10%, 20%, or ranges including and/or spanning the aforementioned values.

In some embodiments, as disclosed elsewhere herein, the lipid-based particle composition is composed of highly pure ingredients, including but not limited to GMP grade therapeutic agents and/or GMP grade phospholipids. In some embodiments, the composition (and/or one or more ingredients constituting the compositions) is manufactured with high purity, multicompendial ingredients to be at the same standards as pharmaceutical products. In some embodiments, the composition is manufactured using pharmaceutical equipment and documentation to ensure the product is of high quality and consistent from batch to batch.

In some embodiments, advantageously, the nanoparticle delivery systems disclosed herein are reproducibly manufacturable. In some embodiments, the method of manufacture of the compositions avoids the introduction of contaminants (such as metal contamination).

In some embodiments, over 50%, 75%, 90%, 95%, 99% (or ranges spanning and or including the aforementioned values) of the nanoparticles disclosed herein have a particle size of between about 20 to about 500 nm (as measured by zeta sizing (e.g., refractive index)). In some embodiments, over 50%, 75%, 90%, 95%, 99% (or ranges spanning and or including the aforementioned values) of the nanoparticles disclosed herein have a particle size of between about 50 nm to about 200 nm (as measured by zeta sizing (e.g., refractive index)). In some embodiments, over 50%, 75%, 90%, 95%, 99% (or ranges spanning and or including the aforementioned values) of the nanoparticles disclosed herein have a particle size of between about 50 nm to about 150 nm (as measured by zeta sizing (e.g., refractive index)). In some embodiments, over 50%, 75%, 90%, 95%, 99% (or ranges spanning and or including the aforementioned values) of the nanoparticles disclosed herein have a particle size of at least 100 nm (as measured by zeta sizing (e.g., refractive index). In some embodiments, this consistency in size allows predictable delivery to subjects.

In several embodiments, the nanoparticle delivery system described herein offers protection to active compounds against degradation in an aqueous environment for long-term storage. In several embodiments, the composition is well characterized to ensure a consistent product from batch to batch and with long-term stability. In several embodiments, the product stability is routinely tested for appearance, particle size and distribution, zeta potential, residual solvents, heavy metals, active compound concentration, and microbial testing and the values measured using these test methods varies (over a period of at least about 1 month or about 6 months at 25° C. with 60% relative humidity) by less than or equal to about: 1%, 5%, 10%, 20%, 30%, 50%, 100%, 150%, 200%, or ranges including and/or spanning the aforementioned values. In several embodiments, the particle size and/or PDI varies over a period of at least about 1 month or about 6 months (at 25° C. with 60% relative humidity) by less than or equal to about: 1%, 5%, 10%, 20%, 30%, 50%, 100%, 150%, 200%, or ranges including and/or spanning the aforementioned values. As noted elsewhere herein, PDI and size can be measured using conventional techniques disclosed herein. In several embodiments, the active agent (e.g., pharmaceuticals, nutraceuticals, cosmetics, pigments, flavorings, and the like) concentration varies over a period of at least about 1 month or about 6 months (at 25° C. with 60% relative humidity) by less than or equal to about: 1%, 5%, 10%, 15%, 20%, 25%, 30%, 35%, 40%, 45% 50%, or ranges including and/or spanning the aforementioned values. As noted elsewhere herein, PDI and size can be measured using conventional techniques disclosed herein.

In several embodiments, the formulations and/or compositions disclosed herein are stable during sterilization. In several embodiments, the sterilization may include one or more of ozonation, UV treatment, and/or heat treatment. In several embodiments, the particle size and/or PDI after sterilization (e.g., exposure to techniques that allow sterilization of the composition) varies by less than or equal to about: 1%, 5%, 10%, 20%, 30%, 50%, 100%, 150%, 200%, or ranges including and/or spanning the aforementioned values. In several embodiments, the active agent (e.g., pharmaceuticals, nutraceuticals, cosmetics, pigments, flavorings, and the like) concentration after sterilization (e.g., exposure to techniques that allow sterilization of the composition) varies (e.g., drops) by less than or equal to about: 1%, 5%, 10%, 15%, 20%, 25%, 30%, 35%, 40%, 45% 50%, or ranges including and/or spanning the aforementioned values.

In several embodiments, the nanoparticle compositions (including after stabilization) disclosed herein have a shelf life of equal to or greater than 6 months, 12 months, 14 months, 16 months, 18 months, 24 months, or ranges including and/or spanning the aforementioned values. The shelf-life can be determined as the period of time in which there is 95% confidence that at least 50% of the response (active agent(s) concentration or particle size) is within the specification limit. This refers to a 95% confidence interval and when linear regression predicts that at least 50% of the response is within the set specification limit.

In several embodiments, the composition contains preservatives to protect against bacteria, mold, and fungal growth. The product specification is no more than 100 cfu/gram. In several embodiments, over a period of about 1 month, about 6 months, or about 12 months the composition has equal to or not more than: 50 cfu/gram, 10 cfu/gram, 5 efu/gram, 1 efu/gram, 0.1 cfu/gram, or ranges including and/or spanning the aforementioned values. In several embodiments, 1 week at 20° C.-25° C. after a 10⁵-10⁷ colony forming units (cfu)/mL challenge with any one of Staphylococcus aureus, Pseudomonas aeruginosa, Escherichia coli, Candida albicans, and Aspergillus brasiliensis the composition has equal to or not more than: 100 cfu/gram, 50 cfu/gram, 25 cfu/gram, 10 cfu/gram, 5 cfu/gram, 1 cfu/gram, 0.1 cfu/gram, or ranges including and/or spanning the aforementioned values. In several embodiments, 1 week at 20° C.-25° C. after a 10⁵-10⁷ cfu/mL challenge with any one of Staphylococcus aureus, Pseudomonas aeruginosa, Escherichia coli, Candida albicans, and Aspergillus brasiliensis the composition has a log reduction for the bacteria of equal to or greater than: 1, 2, 3, 4, 5, 10, or ranges including and/or spanning the aforementioned values.

The shelf-life can be determined as the period of time in which there is 95% confidence that at least 50% of the response (active agent(s) concentration or particle size) is within a specification limit. A specification limit is a range of measured values in which a quality parameter should be within in order for products to be considered of the same quality when it was initially released. For example, where the active ingredient concentration is 20 mg/mL, a specification limit may be defined as 18 to 22 mg/mL, and during a stability study if the active agent concentration falls below 18 mg/mL due to chemical instability, at that time the product may be considered out of specification.

In several embodiments, the shelf life is determined as a time where the concentration of the active ingredient has changed (e.g., lessened) by less than or equal to 15%, 10%, 5%, 2.5%, or ranges including and or spanning the aforementioned ranges.

In several embodiments, the nanoparticle delivery system described herein offers protection to active compounds against degradation at higher temperatures, such as in hot aqueous environments. Hot aqueous environments can include hot liquids such as a hot beverage. The higher temperatures may be temperatures such as 60° C. or higher. Higher temperatures may include above, at, below, between, or any range of 60° C. to 1500° C., such as 60° C. to 100° C., 70° C. to 85° C., 70° C. to 80° C., 70° C., 75° C., 80° C., 85° C., 95° C., 100° C., 110° C., 120° C., 130° C., 140° C., 150° C. In several embodiments, after a 30 minute period in a hot liquid, the particle size and/or PDI varies by less than or equal to about: 1%, 5%, 10%, 20%, 30%, 50%, 100%, 150%, 200%, or ranges including and/or spanning the aforementioned values. In several embodiments, after a 30 minute period in a hot liquid, the active agent (e.g., pharmaceuticals, nutraceuticals, cosmetics, pigments, flavorings, and the like) concentration drops by less than or equal to about: 1%, 5%, 10%, 15%, or ranges including and/or spanning the aforementioned values.

In some embodiments, the density of the composition is purposefully modified. In some embodiments, the density is approximately 0.7 g/mL, 0.75 g/mL, 0.8 g/mL, 0.85 g/mL, 0.9 g/mL, 0.95 g/mL, 1.0 g/mL, 1.05 g/mL, 1.1 g/mL, 1.15 g/mL, 1.2 g/mL, 1.25 g/mL, 1.3 g/mL, or ranges including and or spanning the aforementioned ranges. In some embodiments, the density of the composition is modified to approximately equal the density of an aqueous solution, a gel, a liquid, a cream, or a lotion. In some embodiments, the density is modified by adjust the ratio of different nanoparticle types (e.g., liposomes, solid lipid nanoparticles, etc.). In some embodiments, the density is modified by adjusting the ratio between a first lipid and second lipid. In some embodiments, the density is modified by adjusting the ratio between a first set of lipids and a second set of lipids. In some embodiments, concentrations of lipids with different chain lengths (e.g. 8 carbon, 10 carbon, 18 carbon lipids) are adjusted to modify the density. Such modifications described for modifying density may also influence API solubility (i.e., encapsulation), physical stability, chemical stability, and/or particle composition.

In several embodiments, the particle is a nanoscale particle (e.g., a nanoparticle). In several embodiments, the particle is a microscale particle (e.g., a microparticle).

In several embodiments, advantageously, the individual particles within the disclosed nanoparticle compositions may not settle or sediment appreciably. In several embodiments, an appreciable amount of the composition (e.g., as viewed by the naked eye) does not settle and/or separate from an aqueous liquid upon standing. In several embodiments, the composition does not appreciably settle or separate from an aqueous liquid upon standing for equal to or at least about 1 day, about 1 week, about 1 month, about 2 months, about 3 months, about 6 months, about 9 months, about 1 year, or ranges including and/or spanning the aforementioned values. In several embodiments, upon standing, the composition remains dispersed in an aqueous liquid for at least about 1 day, about 1 week, about 1 month, about 2 months, about 3 months, about 6 months, about 9 months, about 1 year, or ranges including and/or spanning the aforementioned values. In several embodiments, the homogeneity of the disclosed compositions changes by equal to or less than about: 0.5%, 1%, 5%, 7.5%, 10%, or 15% (or ranges including and/or spanning the aforementioned values) after a period of one week or one month. In this case, homogeneity is observed through images by SEM or cyro-SEM (e.g., the average size of the particles and/or the particle types). In several embodiments, the composition remains dispersed in an aqueous liquid and does not appreciably settle or separate from an aqueous liquid after at least about: 1 minute, 5 minutes, 30 minutes, or an hour in a centrifuge at a centripetal acceleration of at least about 100 m/s, at least about 1000 m/s, or at least about 10,000 m/s. In several embodiments, the composition remains dispersed in an aqueous liquid and does not appreciably settle or separate from an aqueous liquid after at least about: 1 minute, 5 minutes, 30 minutes, or an hour in a centrifuge at a centrifuge speed of 5000 RPM, 10,000 RPM, or 15,000 RPM.

In several embodiments, the average size of the nanoparticles of a composition as disclosed herein is substantially constant and/or does not change significantly over time (e.g., it is a stable nanoparticle). In several embodiments, after formulation and storage for a period of at least about 1 month (30 days), about 3 months (90 days), or about 6 months (180 days) (e.g., at ambient conditions, at 25° C. with 60% relative humidity, or under the other testing conditions disclosed elsewhere herein), the average size of nanoparticles comprising the composition changes less than or equal to about: 1%, 5%, 10%, 20%, or ranges including and/or spanning the aforementioned values.

In several embodiments, the average size of the nanoparticles of a composition as disclosed herein is substantially constant and/or does not change significantly over time (e.g., it is a stable nanoparticle) at higher temperatures, such as temperatures over 70° C. In several embodiments, after formulation and exposure to higher temperatures, the average size of nanoparticles comprising the composition changes less than or equal to about: 1%, 5%, 10%, 20%, or ranges including and/or spanning the aforementioned values. In several embodiments, after formulation and exposure to higher temperatures, the concentration of an active ingredient in the nanoparticles changes less than or equal to about: 1%, 5%, 10%, 20%, or ranges including and/or spanning the aforementioned values.

In several embodiments, the polydispersity index (PDI) of the nanoparticles of a composition as disclosed herein is less than or equal to about: 0.05, 0.10, 0.15, 0.20, 0.25, 0.30, 0.40, 0.50, 0.60, 0.70, 0.80, or ranges including and/or spanning the aforementioned values. In several embodiments, the size distribution of the nanoparticles is highly monodisperse with a polydispersity index of less than or equal to about: 0.05, 0.10, 0.15, 0.20, 0.25, or ranges including and/or spanning the aforementioned values.

In several embodiments, the zeta potential of the nanoparticles of a composition as disclosed herein is less than or equal to about: 1 mV, 3 mV, 4 mV, 5 mV, 6 mV, 7 mV, 8 mV, 10 mV, 20 mV, or ranges including and/or spanning the aforementioned values. In several embodiments, the zeta potential of the nanoparticles is greater than or equal to about:: −40 mV, −35 mV, −30 mV, −25 mV, −20 mV, −15 mV, −10 mV, −5 mV, −3 mV, −1 mV, 0 mV, or ranges including and/or spanning the aforementioned values. In several embodiments, the zeta potential and/or diameter of the particles (e.g., measured using dynamic light scattering or laser diffraction) is acquired using a zetasizer (e.g., a Malvern ZS90 or similar instrument) or mastersizer (Mastersizer 3000 or similar instrument).

In several embodiments, the nanoparticle composition has a pH of less than or equal to about: 2, 3, 4, 5, 6, 6.5, 7, 8, 9, or ranges including and/or spanning the aforementioned values. In several embodiments, the composition has a pH of greater than or equal to about: 2, 3, 4, 5, 6, 6.5, 7, 8, 9, or ranges including and/or spanning the aforementioned values. For example, in several embodiments, the composition has a pH ranging from 2 to 4, 4 to 6, 6 to 8, 2 to 9, 2 to 5, 3 to 7, 4 to 8, 5 to 9, etc.

In several embodiments, multilamellar nanoparticles comprise equal to or at least about 5%, 8%, 9%, 10%, 15%, 25%, 50%, 75%, 85%, 95%, or 100% (or ranges spanning and/or including the aforementioned values). of the particles present in the composition (e.g., the aqueous composition) For example, in some embodiments, between about 5% and about 10% of the particles present are multilamellar. In several embodiments, about 8.6% of the particles present are multilamellar.

In several embodiments, unilamellar nanoparticles comprise equal to or at least about 5%, 8%, 9%, 10%, 15%, 20%, 25%, 50%, 75%, 85%, 95%, or 100% (or ranges spanning and/or including the aforementioned values) of the particles present in the composition (e.g., the aqueous composition). For example, in some embodiments, between about 10% and about 15% of the particles present are unilamellar. In several embodiments, about 12.88% of the particles present are unilamellar.

In several embodiments, emulsion particles comprise equal to or at least about 5%, 8%, 9%, 10%, 15%, 25%, 50%, 60%, 65%, 70%, 75%, 85%, 95%, or 100% (or ranges spanning and/or including the aforementioned values) of the particles present in the composition (e.g., the aqueous composition). For example, in some embodiments, between about 60% to about 75% of the particles present are emulsion particles. In several embodiments, about 69.7% of the particles present are emulsion particles.

In several embodiments, micelle particles comprise equal to or at least about 5%, 8%, 9%, 10%, 15%, 25%, 50%, 60%, 65%, 70%, 75%, 85%, 95%, or 100% (or ranges spanning and/or including the aforementioned values) of the particles present in the composition (e.g., the aqueous composition).

In several embodiments, liposomes comprise equal to or at least about 5%, 8%, 9%, 10%, 15%, 25%, 50%, 60%, 65%, 70%, 75%, 85%, 95%, or 100% (or ranges spanning and/or including the aforementioned values) of the particles present in the composition (e.g., the aqueous composition).

In several embodiments, irregular particles (including particles with lamellar structures and/or bridges) comprise equal to or at least about 1%, 2%, 3%, 4%, 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition). For example, in some embodiments, between about 1% to about 5% of the particles present are irregular particles. In several embodiments, 2.73% are irregular particles.

In several embodiments, combined lamellar and emulsion particles comprise equal to or at least about 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition). For example, in some embodiments, between about 5% to about 6% of the particles present are combined lamellar and emulsion particles. In several embodiments, 6.06% of the particles are combined lamellar and emulsion particles.

In several embodiments, mixed-micelle particles comprise equal to or at least about 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition).

The nanoparticle compositions can comprise combinations of multilamellar nanoparticles, unilamellar nanoparticles, emulsion nanoparticles, micelle nanoparticles, irregular particles, and/or liposomes.

The percentages and/or concentrations of particles present in the composition may be purposefully modified. In some embodiments, the percentage and/or concentration of the particles present in the composition are tailored to the active compound and/or the liquid comprising the particles. Such tailoring may lead to more homogenization and/or dispersion in the liquid. The tailoring may stabilize dispersion in the liquid. Such tailoring may also tailor to specific densities of the compositions. The densities of the compositions can be matched or different from a liquid that the compositions are contacted by or contained within.

In some embodiments, the composition is biased towards one type of nanoparticle, such as solid nanoparticles or liposomes. The composition may be biased by increasing or decreasing the ratio in the composition of lipids that are solid at room temperature to lipids that are liquid at room temperature. Lipids that are liquid at room temperature may comprise or exclude MCT (a mixture of capric and caprylic triglycerides, which may have a ratio of c8:c10 carbon chains of 45:55), a triglyceride of capric acid, natural oils, plant oils, seed oils, or synthetic food-grade oils. Lipids that are solid at room temperature may comprise solubilizers and/or emollients. Lipids that are solid at room temperature may comprise or exclude phosphatidylcholine (such as HSPC) phosphatidylethanolamine, sphingomyelin, triglycerides of oleic acid, and/or triglycerol monooleate. In some embodiments, the concentration of an oil is adjusted, such as the concentration of a triglyceride, a fatty acid, a diglyceride, or a monoglyceride. In some embodiments, the concentration of a sterol, such as cholesterol or a plant sterol, is adjusted. In some embodiments, the composition is biased towards liposomes by increasing the concentration of lipids that are liquid at room temperature. Biasing the composition may alter characteristics of the composition including density, particle composition, solubility, pharmacokinetic properties, or other characteristics described herein.

In some embodiments, the ratio of the phospholipid to the at least one oil other than the at least one phospholipid is 1:0.5 to 1:10. In specific embodiments, the ratio of the phospholipid to the at least one oil other than the at least one phospholipid is 1:0.75 to 1:8. the ratio of the phospholipid to the at least one oil other than the at least one phospholipid is 1:1 to 1:6. the ratio of the phospholipid to the at least one oil other than the at least one phospholipid is 1:1.5 to 1:5. the ratio of the phospholipid to the at least one oil other than the at least one phospholipid is 1:2 to 1:4. the ratio of the phospholipid to the at least one oil other than the at least one phospholipid is 1:2.5 to 1:3.5. the ratio of the phospholipid to the at least one oil other than the at least one phospholipid is 1:3.

Decreasing the concentration of a co-emulsifier that is a liquid at room temperature can bias the outcome of the particles more towards liposomes while increasing the concentration of a co-emulsifier that is a liquid at room temperature can bias the outcome of the particles more towards solid lipid nanoparticles. Also, increasing the concentration of a lipid that is solid at room temperature can bias the outcome of particles more towards liposomes. As non-limiting examples, decreasing concentrations of the co-emulsifier MCT and/or increasing the concentration of hydrogenated soy phosphatidylcholine (HSPC) will bias the outcome of the particles more towards liposomes. As a non-limiting example, substituting MCT with a lipid that is a liquid at room temperature and/or increasing the concentration ratio of HSPC to MCT will bias the outcome of the particles more towards liposomes. As another non-limiting example, substituting HSPC with a lipid that is a solid at room temperature will bias the outcome of the particles more towards solid lipid nanoparticles.

In some embodiments, the composition comprises high purity triglycerides, such as oleic acid and/or conjugated linoleics. The composition may be formulated, such as by changing the composition or concentration of lipids, for specific delivery or specific metabolism. For example, the composition may comprise medium chain triglycerides to bias the composition towards phase 1 liver metabolism. In some embodiments, the composition is formulated for a specific absorption mechanism, such as lymphatic absorption or liver first pass.

Some embodiments pertain to a lipid-based particle composition comprising a nanoparticle comprising an active compound that is of sufficient purity that it exists in a solid and/or powdered state prior to formulation in the nanoparticle composition at a weight percent in the composition ranging from 0.001% to 20%; a phosphatidylcholine at a weight percent in the composition ranging from 2.5% to 20%; a sterol at a weight percent in the composition ranging from 0.2% to 5%; and a medium chain triglyceride at a weight percent in the composition ranging from 1% to 15%. In some embodiments, the composition comprises water at a weight percent in the composition ranging from 60% to about 95%. In some embodiments, the nanoparticles have an average size ranging from about 75 nm to about 175 nm. In some embodiments, upon storage for a period of one month, the average size of the nanoparticles changes by less than about 20%.

In some embodiments, the nanoparticle composition is in the form of liposomes and/or an oil-in-water nano-emulsion. In some embodiments, an appreciable amount of the nanoparticle composition does not settle and/or separate from the water upon standing for a period of at least about 12 hours, 24 hours, 3 days, 5 days, a week, 2 weeks, 3 weeks, 5 weeks, 2 months, 3 months, 6 months, 12 months, 18 months, or 24 months. In some embodiments, the composition is configured such that when concentrated to dryness to afford a powder formulation of nanoparticles, the nanoparticle powder can be reconstituted to provide the nanoparticle composition. In some embodiments, the composition has a Tmax for an active agent of less than 4.5 hours. In some embodiments, upon storage for a period of one month, two months, three months, 6 months, 12 months, 18 months, or 24 months the average size of the nanoparticles changes by less than about 20%. In some embodiments, the polydispersity of the nanoparticles in the composition is less than or equal to 0.15. In some embodiments, upon 90 days of storage at 25° C. and 60% relative humidity, the polydispersity of the nanoparticles changes by less than or equal to 10%. In some embodiments, upon 90 days of storage at 25° C. and 60% relative humidity, the polydispersity of the nanoparticles changes by less than or equal to 0.1. In some embodiments, the composition has a shelf life of greater than 18 months at 25° C. and 60% relative humidity. In some embodiments, upon 90 days of storage at 25° C. and 60% relative humidity, the D90 of the nanoparticles changes less than or equal to 10%. In some embodiments, the composition has a concentration max (Cmax) of 80 ng/ml or greater after an oral dose of 15 mg/kg.

In some embodiments, as disclosed elsewhere herein, the nanoparticle composition is in the form and/or comprises one or more of liposomes, an oil-in-water nano-emulsion (and/or microparticle emulsion), and/or solid lipid particles. In some embodiments, when suspended in water, an appreciable amount of the particles in the composition do not settle and/or do not separate (e.g., upon visual inspection) from the water upon standing for a period of at least about 12 hours. In some embodiments, when suspended in water, the particles remain substantially homogenously distributed in the water upon standing for a period of at least about 12 hours, 24 hours, 3 days, 5 days, a week, 2 weeks, 3 weeks, 5 weeks, 2 months, 3 months, 6 months, 12 months, 18 months, or 24 months. In some embodiments, the nanoparticles have an average size ranging from about 10 nm to about 500 nm. In some embodiments, the composition comprises nanoparticles having an average size of less than or equal to about: 10 nm, 50 nm, 100 nm, 250 nm, 500 nm, 1000 nm, or ranges including and/or spanning the aforementioned values. In some embodiments, the composition comprises microparticles having an average size of less than or equal to about: 1000 nm, 1.5 μm, 2 μm, 3 μm, 5 μm, 10 μm or ranges including and/or spanning the aforementioned values. In some embodiments, the dried powder composition comprises microparticles that form nanoparticles (as disclosed herein) when reconstituted. In some embodiments, these dried powder compositions comprise particles having an average size of less than or equal to about: 250 nm, 500 nm, 1000 nm, 1.5 μm, 2 μm, 3 μm, 5 μm, 10 μm, 50 μm, or ranges including and/or spanning the aforementioned values. In some embodiments, upon storage for a period of one month, the average size of the nanoparticles (or microparticles) increases by less than about 10%.

In some embodiments, the nanoparticle composition is configured such that when concentrated to dryness to afford dry particles (e.g., from any one of the oil-in-water emulsion (e.g., a nanoemulsion or microemulsion), liposome solution, and/or solid lipid particle) as a powder, the dry nanoparticles can be reconstituted to provide a reconstituted particle based solution (e.g., the nanoparticle composition). In some embodiments, when reconstituted, the average size of the nanoparticles increases or decreases by less than about 15%, 20%, 25%, 30%, 35%, 40%, 45%, 50%, 55%, 60%, 65%, 70%, 75%, 80%, 85%, 90%, 95%, and/or by less than about 100%. In some embodiments, to form powders, excipients (and/or additives as disclosed elsewhere herein) may be added to the liposomes, oil-in-water nano-emulsions (and/or microparticle emulsions), and/or a solid lipid particle. In some embodiments, the excipient comprises or excludes a carbohydrate, such as trehalose.

### III. Pharmacokinetic Properties

In several embodiments, as disclosed elsewhere herein, the nanoparticle composition aids in absorption, bioavailability, or other pharmacokinetic properties of the active compound when administered to an individual, including by orally ingestion. In several embodiments, the compositions disclosed herein allow the active compound to be delivered to and/or absorbed through the gut. As disclosed elsewhere herein, some embodiments pertain to the use of the nanoparticle based nanodelivery system to protect the active compound from degradation and/or precipitation in a solution comprising the active compound (e.g., in an aqueous composition for administration to a subject). In several embodiments, use of the delivery systems, including the nanoparticles, disclosed herein result in improved bioavailability and/or absorption rate. For instance, in some embodiments, the Cmax of an active compound is increased using a disclosed embodiment, the Tmax of an active compound is decreased using an embodiment as disclosed herein, and/or the AUC of an active compound is increased using a disclosed embodiment.

In several embodiments, the pharmacokinetic outcomes disclosed elsewhere herein (Cmax, Tmax, AUC, t_{1/2}, etc.) can be achieved using aqueous nanoparticle compositions or powdered nanoparticle compositions (e.g., where the powder is supplied by itself, in a gel capsule, as an additive to food, etc.).

In several embodiments, the Cmax of the active agent is increased using the disclosed embodiments relative to other delivery vehicles (e.g., after administration to a subject). In several embodiments, the Cmax is increased relative to the active agent (e.g., pharmaceuticals, nutraceuticals, and the like) alone or comparator embodiments (e.g., oil-based products) by equal to or at least about: 15%, 20%, 50%, 100%, 150%, 200%, or ranges including and/or spanning the aforementioned values. In several embodiments, the active agent Cmax is increased (relative to a comparator oil-based product) by equal to or at least about: 5%, 10%, 20%, 30%, 50%, 100%, or ranges including and/or spanning the aforementioned values. In several embodiments, the active agent Cmax is increased (relative to a comparator oil-based product) by equal to or at least about: 10 ng/mL, 20 ng/mL, 30 ng/mL, 40 ng/mL, 50 ng/mL, 60 ng/mL, 70 ng/mL, 80 ng/mL, 90 ng/mL, or ranges including and/or spanning the aforementioned values.

In several embodiments, after a dose of 15 mg of active agent (e.g., pharmaceuticals, nutraceuticals, and the like) provided in an embodiment as disclosed herein to a subject (e.g., a mini-pig, human, etc.), the Cmax of the active agent is equal to or at least about: 0.5 μg/L, 1 μg/L, 2 μg/L, 3 μg/L, 4 μg/L, 5 μg/L, 6 μg/L, or ranges including and/or spanning the aforementioned values. In several embodiments, after a dose of 15 mg/kg of active agent (e.g., pharmaceuticals, nutraceuticals, and the like) provided in an embodiment as disclosed herein to a subject, the Cmax is equal to or at least about: 40 ng/mL, 50 ng/mL, 60 ng/mL, 70 ng/mL, 80 ng/mL, 90 ng/mL, 100 ng/mL, 150 ng/mL, 200 ng/mL, or ranges including and/or spanning the aforementioned values.

In several embodiments, the Cmax for a disclosed embodiment is increased relative to an equal dose of an active agent (e.g., pharmaceuticals, nutraceuticals, and the like) in an oil-based comparator vehicle. In several embodiments, the Cmax for a disclosed embodiment is increased relative to an oil-based comparator vehicle by equal to or at least about: 15%, 20%, 50%, 100%, 150%, 200%, or ranges including and/or spanning the aforementioned values. In several embodiments, these pharmacokinetic results can be achieved using aqueous compositions or powdered compositions (where the powder is supplied by itself, in a gel capsule, as an additive to food, etc.). In some instances, the Cmax using a disclosed embodiment is 1.25 times higher than when using a comparator delivery system (e.g., the Cmax of the comparator×1.25). In some instances, the Cmax using a disclosed embodiment is equal to or at least about 1.25 times higher, 1.5 times higher, 2 times higher, 3 times higher (or ranges including or spanning the aforementioned values) than when using a comparator delivery system.

In several embodiments, the Tmax for an active agent using a disclosed embodiment is shortened relative to other vehicles. In several embodiments, after a dose of active agent (e.g., pharmaceuticals, nutraceuticals, and the like) provided in an embodiment as disclosed herein to a subject as disclosed herein, the Tmax is equal to or at less than about: 30 minutes, 1 hours, 2 hours, 3 hours, 4 hours, 4.5 hours, 5 hours, 5.5 hours, 6 hours, 6.5 hours, 7 hours, 8 hours, or ranges including and/or spanning the aforementioned values. In several embodiments, after a dose of 15 mg/kg of active agent provided in an embodiment as disclosed herein to a subject, the Tmax is equal to or at less than about: 30 minutes, 1 hours, 2 hours, 3 hours, 4 hours, 5 hours, 5.5 hours, 6 hours, 6.5 hours, 7 hours, 8 hours, or ranges including and/or spanning the aforementioned values. In several embodiments, after a dose of active agent provided in an embodiment as disclosed herein to a subject, the Tmax is between about 4 hours and about 6.5 hours or between about 3 hours and about 7 hours. In several embodiments, after a dose of 15 mg of active agent provided in an embodiment as disclosed herein to a human patient, the Tmax is equal to or less than about: 3 hours, 4 hours, 5 hours, 6 hours, 7 hours, 8 hours, or ranges including and/or spanning the aforementioned values.

In several embodiments, after of a dose of active agent (e.g., pharmaceuticals, nutraceuticals, and the like) (e.g., a 15 mg/kg dose) provided in an embodiment as disclosed herein to a subject (e.g., a mini-pig, human, etc.), the AUC is equal to or at least about: 50 ng/mL*hr, 100 ng/mL*hr, 200 ng/mL*hr, 300 ng/mL*hr, 400 ng/mL*hr, 450 ng/mL*hr, 500 ng/mL*hr, 550 ng/mL*hr, 600 ng/mL*hr, 650 ng/mL*hr, 700 ng/mL*hr, 800 ng/mL*hr, 1000 ng/mL*hr, or ranges including and/or spanning the aforementioned values.

In several embodiments, the half-life for an active agent (e.g., pharmaceuticals, nutraceuticals, and the like) (t₁12) in vivo using a disclosed embodiment can be shorter relative to other vehicles. In several embodiments, after a dose of active agent (e.g., pharmaceuticals, nutraceuticals, and the like) provided in an embodiment as disclosed herein to a subject as disclosed herein, the t_{1/2} of active agent is equal to or at less than about: 4 hours, 5 hours, 5.5 hours, 6 hours, 6.5 hours, or ranges including and/or spanning the aforementioned values. In several embodiments, after a dose of active agent provided in an embodiment as disclosed herein to a subject, the t_{1/2} of active agent is between about 4 hours and about 6.5 hours or between about 3 hours and about 7 hours. In several embodiments, the t_{1/2} for a disclosed embodiment is decreased relative to an active agent alone or an oil-based comparator vehicle by equal to or at least about: 15%, 20%, 50%, 100%, 150%, 200%, or ranges including and/or spanning the aforementioned values. In several embodiments, the t_{1/2} of active agent for a disclosed embodiment is decreased relative to the active alone or an oil-based comparator vehicle by equal to or at least about: 15 minutes, 30 minutes, 45 minutes, 1 hour, 2 hours, or ranges including and/or spanning the aforementioned values. In some instances, the t₁/2 is a fraction of that achieved using a comparator delivery system. In some instances, the time to t_{1/2} using a disclosed embodiment is 0.5 times, 0.7 times, 0.8 times, 0.9 times, or 0.95 times the t_{1/2} of a comparator delivery system (or ranges including or spanning the aforementioned values).

In several embodiments, as disclosed elsewhere herein, the nanoparticle composition is stable. In several embodiments, for example, after formulation (e.g., in water at concentrations disclosed elsewhere herein) and storage for a period of at least about 1 month, 3 months, 6 months, 12 months, 18 months, 24 months, or ranges including or spanning the aforementioned values, the polydispersity of the nanoparticles changes less than or equal to about: 1%, 5%, 10%, 20%, 30%, 50%, 75%, 100%, or ranges including and/or spanning the aforementioned values. In several embodiments, after formulation (e.g., in water at concentrations disclosed elsewhere herein) and storage for a period of at least about 1 month, 3 months, 6 months, 12 months, 18 months, 24 months, or ranges including or spanning the aforementioned values, the soluble fraction of active agent (e.g., pharmaceuticals, nutraceuticals, cosmetics, pigments, flavorings, and the like) in the formulation changes less than or equal to about: 1%, 5%, 10%, 20%, 30%, 50%, 75%, or ranges including and/or spanning the aforementioned values. In several embodiments, after formulation and storage for a period of at least about 1 month, 3 months, 6 months, 12 months, 18 months, 24 months, or ranges including or spanning the aforementioned values, (e.g., at ambient conditions, at 25° C. with 60% relative humidity, or under the other testing conditions disclosed elsewhere herein), the PDI of nanoparticles comprising the composition changes by less than or equal to about: 1%, 5%, 10%, 20%, 30%, 50%, 75%, 100%, or ranges including and/or spanning the aforementioned values. In several embodiments, after formulation and storage for a period of at least about 1 month, 3 months, 6 months, 12 months, 18 months, 24 months, or ranges including or spanning the aforementioned values, (e.g., at ambient conditions, at 25° C. with 60% relative humidity, or under the other testing conditions disclosed elsewhere herein), the PDI of nanoparticles comprising the composition changes by less than or equal to about: 0.05, 0.1, 0.2, 0.3, 0.4, or ranges including and/or spanning the aforementioned values.

In several embodiments, when exposed to simulated gastric fluid (e.g., at a concentration of 20 mg/mL), the particle size of the nanoparticles of a composition as disclosed herein does not change and/or changes less than 5% during a period of greater than or equal to about: 1 hour, 2 hours, 3 hours, 4 hours, 5 hours, 6 hours, 10 hours, or ranges including and/or spanning the aforementioned values. In several embodiments, when exposed to simulated intestinal fluid (e.g., at a concentration of 20 mg/mL), the particle size of the nanoparticles disclosed herein does not change and/or changes less than 5% during a period of greater than or equal to about: 1 hour, 2 hours, 3 hours, 4 hours, 5 hours, 6 hours, 10 hours, or ranges including and/or spanning the aforementioned values. In several embodiments, after formulation (e.g., at a concentration of 20 mg/mL) and storage in simulated gastric fluid for a period of at least about 1 hour or about 2 hours (e.g., at 37° C., or under the other testing conditions disclosed elsewhere herein), the average particle size of nanoparticles comprising the composition changes by less than or equal to about: 1%, 5%, 10%, 20%, 50%, 75%, 100%, or ranges including and/or spanning the aforementioned values. In several embodiments, after formulation (e.g., at a concentration of 20 mg/mL) and storage in simulated gastric fluid for a period of at least about 1 hour, about 2 hours, about 3 hours, or about 4 hours (e.g., at 37° C. or under the other testing conditions disclosed elsewhere herein), the PDI of nanoparticles comprising the composition changes by less than or equal to about: 1%, 5%, 10%, 20%, 30%, 50%, 75%, 100%, or ranges including and/or spanning the aforementioned values. In several embodiments, after formulation (e.g., at a concentration of 20 mg/mL) and storage in simulated gastric fluid for a period of at least about 1 hour or about 2 hours (e.g., at 37° C. or under the other testing conditions disclosed elsewhere herein), the PDI of nanoparticles comprising the composition changes by less than or equal to about: 0.01, 0.05, 0.1, 0.2, 0.3, 0.4, or ranges including and/or spanning the aforementioned values. In several embodiments, after formulation (e.g., at a concentration of 20 mg/mL) and storage in simulated intestinal fluid for a period of at least about 1 hour or about 2 hours (e.g., at 37° C., or under the other testing conditions disclosed elsewhere herein or under the other testing conditions disclosed elsewhere herein), the average particle size of nanoparticles comprising the composition changes by less than or equal to about: 1%, 5%, 10%, 20%, 50%, 75%, 100%, or ranges including and/or spanning the aforementioned values. In several embodiments, after formulation (e.g., at a concentration of 20 mg/mL) and storage in simulated intestinal fluid for a period of at least about 1 hour, about 2 hours, about 3 hours, or about 4 hours (e.g., at 37° C. or under the other testing conditions disclosed elsewhere herein), the PDI of nanoparticles comprising the composition changes by less than or equal to about: 1%, 5%, 10%, 20%, 30%, 50%, 75%, 100%, 150%, or ranges including and/or spanning the aforementioned values. In several embodiments, after formulation (e.g., at a concentration of 20 mg/mL) and storage in simulated intestinal fluid for a period of at least about 1 hour, about 2 hours (e.g., at 37° C. or under the other testing conditions disclosed elsewhere herein), the PDI of nanoparticles comprising the composition changes by less than or equal to about: 0.01, 0.05, 0.1, 0.2, 0.3, 0.4, or ranges including and/or spanning the aforementioned values.

In several embodiments, the composition particle size remains consistent (a size change of less than or equal to about: 0%, 0.5%, 1%, 2%, 3%, 5%, 10%, 20%, 30%, 50%, 75%, 100%, or ranges including and/or spanning the aforementioned values) for a period of at least about 30 days when stored at room temperature, refrigeration, and up to 40° C. In several embodiments, the active agent (e.g., pharmaceuticals, nutraceuticals, cosmetics, pigments, flavorings, and the like) concentration in the composition remains consistent (a loss of less than or equal to about: 0.5%, 1%, 2%, 3%, 5%, 10%, 20%, 30%, 50%, or ranges including and/or spanning the aforementioned values) for a period of at least about 30 days, 60 days, 90 days, or 120 days when stored at room temperature, refrigeration, and up to 40° C. In several embodiments, when stored at room temperature, refrigeration, and up to 40° C., the composition is stable (e.g., the particle size or active agent concentration in the nanoparticles remains consistent and/or has a change of less than or equal to about: 0.5%, 1%, 2%, 5%, 10%, 20%, 30%, 50%, or ranges including and/or spanning the aforementioned values) for a period of at least about: 2 weeks, 30 days, 2 months, 3 months, 6 months, 9 months, 1 year, or ranges including and/or spanning the aforementioned measures of time.

In several embodiments, when exposed to higher temperatures, e.g., above 70° C., the average size of nanoparticles comprising the composition changes less than or equal to about: 1%, 5%, 10%, 20%, or ranges including and/or spanning the aforementioned values. In several embodiments, after exposure to higher temperatures, the concentration of an active ingredient in the nanoparticles changes less than or equal to about: 1%, 5%, 10%, 20%, or ranges including and/or spanning the aforementioned values.

In several embodiments, the method of using the nanoparticle composition and/or of treating a subject with the nanoparticle composition includes administering to a subject in need of treatment (e.g., orally, topically, etc.) an effective amount of the composition. In several embodiments, the composition (e.g., delivery system) improves the stability of the active agent (e.g., pharmaceuticals, nutraceuticals, cosmetics, pigments, flavorings, and the like) after ingestion where the composition is exposed to the stomach and/or intestines in an aqueous environment with harsh pH conditions. In several embodiments, the bioavailability of the active agent (e.g., pharmaceuticals, nutraceuticals, cosmetics, pigments, flavorings, and the like) relative to the initial administered dose is greater than or equal to about: 10%, 20%, 50%, 75%, or ranges including and/or spanning the aforementioned values. In several embodiments, using the disclosed compositions, the oral bioavailability of the active agent (e.g., pharmaceuticals, nutraceuticals, cosmetics, pigments, flavorings, and the like) delivered (as measured using AUC) is higher using an embodiment disclosed herein relative to oral delivery of the active alone. In several embodiments, the oral bioavailability is improved over the active alone by greater than or equal to about: 10%, 50%, 75%, 100%, 200%, or ranges including and/or spanning the aforementioned values.

### III. Manufacturing Compositions

As disclosed elsewhere herein, some embodiments pertain to methods of preparing nanoparticle compositions. In several embodiments, the composition is prepared by adding together one or more of an active agent, a lipid source, a saponin, phospholipid, optionally a preservative, optionally a flavoring agent, or combinations of any of the foregoing, and optionally water. In several embodiments, the composition is prepared using high sheer inline mixing (including for example via Silverson). In several embodiments, the composition is prepared using an overhead mixer (such as, for example, a IKA and/or Silverson). In several embodiments, the composition is prepared using high pressure homogenization. In several embodiments, the composition is prepared using microfluidization. In several embodiments, the composition is prepared using sonication. In several embodiments, the composition is prepared using mechanical stirring. In several embodiments, the composition is prepared using coacervation. In several embodiments, the composition is prepared using solvent precipitation. In several embodiments, the composition is prepared using hot melt extrusion (HME) tablet manufacturing. In several embodiments, the composition is prepared using one or more of the techniques or steps described above or elsewhere herein together. In several embodiments, the composition is prepared with methods excluding any one or more of these steps or techniques.

In several embodiments, the nanoparticle compositions herein are lyophilized (e.g., to provide a powder). In several embodiments, where lyophilization is used to prepare a mixed micelle-based powder, one or more lyoprotectant agents may be added. In several embodiments, an individual lyoprotectant agent may be present at a dry wt. % equal to or less than the dry weight of the lipophilic ingredients. In several embodiments, the lyoprotectant agent(s) (collectively or individually) may be present at a dry wt. % equal to or less than about: 5%, 10%, 15%, 20%, 30%, 40%, 50%, 60%, or ranges including and/or spanning the aforementioned values. In several embodiments, the lyoprotectant agent(s) (collectively or individually) may be present at a wet wt. % of equal to or less than about: 2.5%, 5%, 7.5%, 10%, 12.5%, 15%, 20%, 30%, or ranges including and/or spanning the aforementioned values. In several embodiments, the lyoprotectant is selected from the group consisting of lactose, dextrose, trehalose, arginine, glycine, histidine, and/or combinations thereof. In several embodiments, the nanoparticle compositions herein are spray dried (e.g., to provide a powder). In several embodiments, the nanoparticle compositions are spray dried and not lyophilized. In some embodiments, the nanoparticle composition is spray dried, fluid bed dried, desiccated, and/or lyophilized.

Nanoparticle formulations containing active agents can be formed into spray dried powders. Formulations containing active agents alone with varying amounts of various lipids and other excipients can be prepared using a solvent-free manufacturing process. Water-soluble components can be dissolved in water at 65° C. with magnetic stirring. High shear mixing can be applied at 65° C. and lipids and excipients can be added. High shear mixing can be maintained until a stable suspension is formed. The suspension can then optionally be microfluidized for 5 passes using an MP110 microfluidizer at 30,000 psi. Following microfluidization, formulations can be diluted with an equal volume containing the excipient type such that the excipient is at the stated final concentration. Up to 25% ethanol (e.g., 10%, 15%, 20%, 25%) can be added to the formulation and mixed to a homogenous solution prior to spray drying. Formulations can be spray dried on a Buchi B290 benchtop spray dryer. The inlet temperature can be set at 125° C., the aspirator set to 100%, the pump rate set to 10%, and nitrogen flow set to 60 mmHg. Powder can be collected and measured for residual moisture, and recovered yield can be calculated.

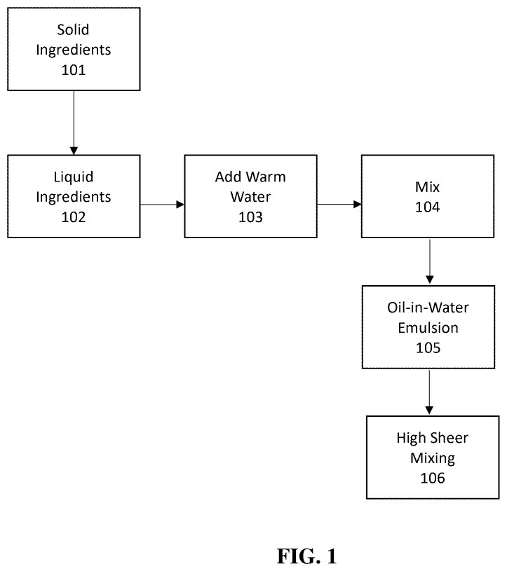

In several embodiments, the composition is prepared by forming a lipid-in-oil emulsion. In several embodiments, an oil-in-water emulsion can be prepared without the use of organic solvents as shown in FIG. 1 (e.g., in an organic solvent-free method). In several embodiments, solid ingredients 101 are added and dissolved into liquid ingredients 102. In several embodiments, the lipid (e.g., phosphatidylcholine, including phosphatidylcholine of any purity disclosed herein) can be added with mixing. In several embodiments, when a well dispersed lipid phase is formed after mixing, the addition of water 103 (e.g., having a temperature of equal to or at least about: 10° C., 20° C., 30° C., 40° C., 50° C., 60° C., 80° C., or ranges including and/or spanning the aforementioned values) and additional mixing 104 achieves an oil-in-water emulsion 105. In several embodiments, the oil-in-water emulsion is then subject to high-shear mixing to form nanoparticle compositions. In several embodiments, high-shear mixing 106 is performed using a high shear dispersion unit or an in-line mixer can be used to prepare the emulsions. In several embodiments, the particles can be made by solvent evaporation and/or solvent precipitation. In several embodiments, high sheer mixing is not required.

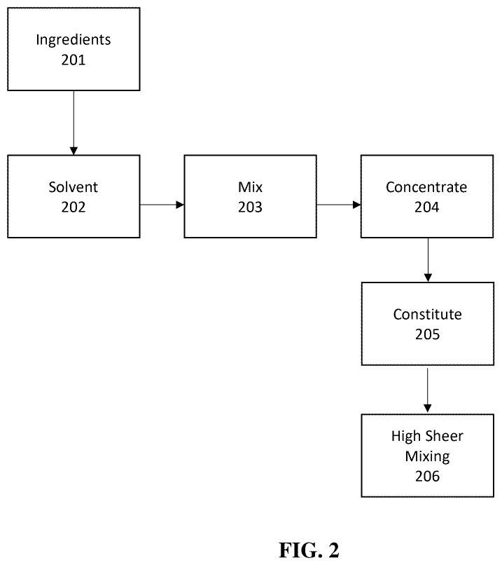

In several embodiments, as shown in FIG. 2, the lipid-in-oil emulsion is formed by dissolving ingredients 201, such as, one or more of a lipid (e.g., phosphatidylcholine, including phosphatidylcholine or any purity disclosed herein), a saponin, one or more active compounds, and/or a phospholipid in a solvent 202. In several embodiments, the solvent can include one or more organic solvents, including but not limited to, ethanol, chloroform, and/or ethyl acetate. In several embodiments, the solvents are class II solvents, class III solvents (e.g., at least class II and/or class III by the ICH Q3C standard), or mixtures thereof. In several embodiments, the solution of ingredients and solvent is dried 203. In several embodiments, after drying, the ingredients are provided as lipids and or liposomes as a thin film. In several embodiments, the solvent is removed from the composition by subjecting the solution to heat under vacuum to promote evaporation. In several embodiments, the film may further be dried under nitrogen gas. In several embodiments, the lipid film is hydrated 205 with warm aqueous solution to form an oil-in-water emulsion. In several embodiments, high-shear mixing is performed 206 using a high shear dispersion unit or an in-line mixer can be used to prepare the emulsions.

In some embodiments, the dried composition, comprising the nanoparticle, is reconstituted. In some embodiments, the nanoparticle composition, such as the percentage and/or concentration of the types of nanoparticles, may change when dried. In some embodiments, the nanoparticle composition, such as the percentage and/or concentration of the types of nanoparticles, may change when reconstituted. In some embodiments, the nanoparticle composition, such as the percentage and/or concentration of the types of nanoparticles, may not change when dried. In some embodiments, the nanoparticle composition, such as the percentage and/or concentration of the types of nanoparticles, may not change when reconstituted.

In several embodiments, as disclosed elsewhere herein, the lipid-in-water emulsion is subject to high pressure homogenization using a microfluidizer and/or a high pressure homogenizer. In several embodiments, high sheer mixing can be used to reduce the particle size. In several embodiments, the oil-in-water emulsion is processed to a nanoparticle (e.g., about 20 to about 500 nm, etc.) using the microfluidizer or other high sheer processes. In several embodiments, the oil-in-water emulsion is processed to a nanoparticle having a size from about 80 nm to 180 nm in diameter or about 100 nm to about 150 nm in diameter. In several embodiments, high sheer mixing is not used.

In several embodiments, the lipid-in-water emulsion is not passed through the microfluidizer and/or a high pressure homogenizer. In several embodiments, the lipid-in-water emulsion is passed through the microfluidizer a plurality of times (e.g., equal to or at least 1 time, 2 times, 3 times, 4 times, 5 times, 10 times, or ranges including and/or spanning the aforementioned values). In several embodiments, the emulsion is passed through the microfluidizer at a pressure of equal to or less than about: 5,000 PSI, 15,000 PSI, 20,000 PSI, 25,000 PSI, 30,000 PSI, or ranges including and/or spanning the aforementioned values. In several embodiments, the emulsion is passed through the microfluidizer at a temperature of equal to or at least about: 30° C., 40° C., 50° C., 65° C., 80° C., or ranges including and/or spanning the aforementioned values. In several embodiments, the emulsion is passed through the microfluidizer at least about room temperature (e.g., about 20° C. or about 25° C.) and/or without any heating and/or temperature control. In several embodiments, the emulsion is passed through the microfluidizer at a temperature of equal to or less than about 80° C. In several embodiments, the microfluidizer includes an interaction chamber consisting of 75 μm to 200 μm pore sizes and the emulsion is passed through this chamber. In several embodiments, the pore size of the microfluidizer are less than or equal to about: 75 μm, 100 μm, 150 μm, 200 μm, 250 μm, 300 μm, or ranges including and/or spanning the aforementioned values. In several embodiments, the nanoparticle composition is prepared by high shear mixing, sonication, or extrusion.

In several embodiments, after preparation, the nanoparticle composition is characterized by an ability to pass through a 0.2 μm filter while preserving the nanoparticle structure (e.g., a change in average nanoparticle size of no greater than 10 nm, 20 nm, or 30 nm). In several embodiments, after passage through a 0.2 μm there is a change in average diameter of the particles of equal to or at less than about: 1%, 5%, 10%, 20%, or ranges including and/or spanning the aforementioned values. In several embodiments, after passage through a 0.2 μm there is a change in PDI of the particles of equal to or at less than about: 1%, 5%, 10%, 20%, or ranges including and/or spanning the aforementioned values.

In several embodiments, as disclosed elsewhere herein, the active nanoparticle composition imparts solubility to hydrophobic active agents in a delivery system that is easily dispersible in aqueous solutions.

Some embodiments, as disclosed elsewhere herein, pertain to a method of manufacturing a lipid-based particle composition. In some embodiments, one or more active compounds is mixed with one or more lipophilic components of the composition to provide a solution. In some embodiments, one or more lipid components (including those that are not phospholipids) are added. In some embodiments, one or more sterols are added. In some embodiments, one or more phospholipids are added. In some embodiments, one or more flavoring and/or preservatives are added. In some embodiments one or more saponin is added. In some embodiments, water is added. In some embodiments, the lipophilic ingredients are combined and the hydrophilic ingredients are combined separately. In some embodiments the lipophilic ingredients are then added to the hydrophilic ingredients. In some embodiments, the solution is passed through a microfluidizer and/or a high sheer homogenizer. In some embodiments, the process affords a particle composition.

In several embodiments, advantageously, the nanoparticle delivery systems disclosed herein are reproducibly manufacturable.

In several embodiments, the active compound may be preprocessed. In several embodiments, pre-processing allows greater encapsulation efficiency and stability by precipitating “other” material away from the active compound. In several embodiments, pre-processing may provide enhanced consumer experience due to less impurity/non-actives in the formulation. In several embodiments, “salt” byproducts are removed directly/indirectly by various extraction techniques. In several embodiments, pre-processing techniques include ethanol and co-solvent precipitation, filtration, activated charcoal soaking+filtration; chromatography, etc. By way of removing solvent such as ethanol, rotary evaporation may be used. Other forms of solvent removal are possible. Other forms of removing active and non-active material are disclosed elsewhere herein (including solvent extraction, acid/base titration, CO₂ extraction (both supercritical and non), cryogenic ethanol extraction, etc. In several embodiments, pre-processing (prior to use in the composition) allows a formulator to use any kind of plant extract or biomass regardless of prior extraction techniques.

Other appropriate methods of salt separation and/or purification during preprocessing may include one or more of size exclusion, ion exchanger in presence of neutral organic compounds may also be a suitability method of purifications, evaporation and distillation membrane extraction, liquid-liquid extraction, solid phase extraction, immobilized liquid extraction, sorptive extraction, charged resin, and/or gel filtration. In several embodiments, pharmaceutical acceptable applications may also be used such as high purity filter media such as diatomite filter aids. Example may include Celpure®, AW Celite®, Harborlite®, etc. In several embodiments, dialysis methods may also be used. In several embodiments, activated charcoal is used to treat a solution comprising the extract. In several embodiments, acidic conditions may be used for preprocessing. In several embodiments, polar, aprotic solvent like DMSO may also be used to solubilize hydrophobic substances like alkaloids during preprocessing. In several embodiments, DMSO and may be acidified or basified to maximize solubility and stability of alkaloids and other compounds.

One or more benefits of the preprocessing step may include better formulations, better encapsulation, purer compositions, industrial isolation for raw material, certified reference material/standards, manufacturer of finished intermediate and/or raw materials, or other benefits.

In several embodiments, the formulation is provided as a suspension type. In several embodiments, formulations containing saponins will provide greater regulatory certainty if active agents are considered controlled substance and/or drugs.

### IV. Administration of Compositions

Certain embodiments of the disclosure relate to compositions and methods of administering the compositions.

The compositions, which may comprise the nanoparticles and active compounds of the disclosure, may be administered via a route of administration. In some embodiments, the composition is administered by more than one route of administration. In some embodiments, the composition is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

In some embodiments, the composition is administered at a dose of between 1 mg/kg and 5000 mg/kg. In some embodiments, the composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

The quantity to be administered, both according to number of treatments and dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the composition is one which can provide a sample level of the active compound at a concentration of about 1 μM to 150 PM. In another embodiment, the effective dose provides a sample level of about 4 μM to 100 PM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following sample level of the agent that results from an active agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. The sample level may be analyzed from any biological sample, such as a blood sample, urine sample, skin sample, saliva sample, or the like. In certain embodiments, the active agent that is administered to a subject is metabolized in the body to a metabolized active agent, in which case the blood levels may refer to the amount of that agent. Alternatively, to the extent the active agent is not metabolized by a subject, the blood levels discussed herein may refer to the unmetabolized active agent.

Precise amounts of the active composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/mL or mM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The compositions can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In certain embodiments, the form must be sterile and must be fluid to the extent that it may be easily injected. It also should be stable under the conditions of manufacture and storage and must be preserved against the contaminating action of microorganisms, such as bacteria and fungi.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

As disclosed elsewhere herein, some embodiments pertain to methods of treating a subject. In several embodiments, the method of treating comprises selecting patient for treatment. In several embodiments, the method of threating comprises administering to the patient an effective amount of a formulation comprising a nanoparticle composition comprising an active agent (e.g., pharmaceuticals, nutraceuticals, cosmetics, pigments, flavorings, and the like).

In several embodiments, compositions as described herein may be used to induce at least one effect, e.g. therapeutic effect, that may be associated with at least one active agent (e.g., pharmaceuticals, nutraceuticals, and the like), which is capable of inducing, enhancing, arresting or diminishing at least one effect, by way of treatment or prevention of unwanted conditions or diseases in a subject. As disclosed elsewhere herein, the at least one active agent may be selected amongst therapeutic agents, such as agents capable of inducing or modulating a therapeutic effect when administered in a therapeutically effective amount.

In several embodiments, the compositions disclosed herein can be used in methods of treatment and can be administered to a subject having a condition to be treated. In several embodiments, the subject is treated by administering an effective amount of a composition as disclosed herein to the subject.

In several embodiments, the disease or condition to be treated via administration of a composition as disclosed herein may include one or more of opioid withdrawal, pain relief, anxiety relief, depression, insomnia, inflammation, fever, fatigue, muscle aches, etc. In several embodiments, the nanoparticle composition (e.g., those including one or more active agents) is provided for use in treating a condition selected from pain associated disorders (as an analgesic), inflammatory disorders and conditions (as anti-inflammatory), appetite suppression or stimulation (as anoretic or stimulant), symptoms of vomiting and nausea (as antiemetic), intestine and bowl disorders, disorders and conditions associated with anxiety (as anxiolytic), disorders and conditions associated with psychosis (as antipsychotic), disorders and conditions associated with seizures and/or convulsions (as antiepileptic or antispasmodic), sleep disorders and conditions (as anti-insomniac), disorders and conditions which require treatment by immunosuppression, disorders and conditions associated with elevated blood glucose levels (as antidiabetic), disorders and conditions associated with nerve system degradation (as neuroprotectant), inflammatory skin disorders and conditions (such as psoriasis), disorders and conditions associated with artery blockage (as anti-ischemic), disorders and conditions associated with bacterial infections, disorders and conditions associated with fungal infections, proliferative disorders and conditions, disorders and conditions associated with inhibited bone growth, post trauma disorders, and others.

The active agent (substance, molecule, element, compound, entity, or a combination thereof) may be selected amongst therapeutic agents, such as agents capable of inducing or modulating a therapeutic effect when administered in a therapeutically effective amount, and non-therapeutic agents, such as agents which by themselves do not induce or modulate a therapeutic effect but which may endow the pharmaceutical composition with a selected desired characteristic.

In several embodiments, a nanoparticle compositions as disclosed herein may be selected to treat, prevent or ameliorate any pathology or condition. In several embodiments, administering of a therapeutic amount of the composition or system described herein, whether in a concentrate form or in a diluted formulation form, is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

In several embodiments, as mentioned elsewhere herein, the compositions disclosed herein, may be provided in a number of different forms for administration and/or ingestion. In several embodiments, including when provided in a ready-to-drink beverage, the compositions are stable during ozonation sterilization, UV sterilization, heat sterilization, filtration sterilization, and/or gamma irradiation during beverage preparation and packaging. In several embodiments, the particle size and/or PDI after sterilization (e.g., exposure to techniques that allow sterilization of the composition) varies by less than or equal to about: 1%, 5%, 10%, 20%, 30%, or ranges including and/or spanning the aforementioned values. In several embodiments, the active agent (e.g., pharmaceuticals, nutraceuticals, cosmetics, pigments, flavorings, and the like) concentration after sterilization (e.g., exposure to techniques that allow sterilization of the composition) drops by less than or equal to about: 1%, 5%, 10%, 15%, or ranges including and/or spanning the aforementioned values. In several embodiments, including after stabilization, the beverages comprising nanoparticle compositions have a shelf life of equal to or greater than 6 months, 12 months, 14 months, 16 months, 18 months, 19 months, 24 months, or ranges including and/or spanning the aforementioned values.

In several embodiments, the compositions are provided in a sterilized beverage. In several embodiments, the sterilized beverage may be a cold beverage (e.g., juices, sports drinks, energy drinks, protein drinks, nutritional drinks, sodas, etc.). In several embodiments, the cold beverage may be a carbonated beverage. In several embodiments, the cold beverage may be an alcoholic beverage. In several embodiments, the compositions may be provided in hot beverages (e.g., coffee, tea, etc.). In several embodiments, after a 30 minute period in a hot beverage, the particle size and/or PDI varies by less than or equal to about: 1%, 5%, 10%, 20%, 30%, or ranges including and/or spanning the aforementioned values. In several embodiments, after a 30 minute period in a hot beverage, the active agent (e.g., pharmaceuticals, nutraceuticals, cosmetics, pigments, flavorings, and the like) concentration drops by less than or equal to about: 1%, 5%, 10%, 15%, or ranges including and/or spanning the aforementioned values.

Several embodiments also encompass methods for administering the disclosed compositions. Multiple techniques of administering the nanoparticle compositions as disclosed herein exist including, but not limited to, oral, sublingual, buccal, rectal, topical, vaginal, aerosol, injection and parenteral delivery, including intramuscular, subcutaneous, intravenous, intramedullary injections, intrathecal, direct intraventricular, intraperitoneal, intranasal, and intraocular injections. In several embodiments, administration is performed through oral pathways, which administration includes administration in an emulsion, capsule, tablet, film, chewing gum, suppository, granule, pellet, spray, syrup, or other such forms. As further examples of such modes of administration and as further disclosure of modes of administration, disclosed herein are various methods for administration of the disclosed compositions including modes of administration through intraocular, intranasal, and intraauricular pathways. In several embodiments, oral formulations may comprise of DMSO and NMP.

In several embodiments, the nanoparticles (or compositions comprising them) may be used to deliver active compounds to a biomass. Thus, the biomass may be fortified with extracts (as disclosed elsewhere herein). In several embodiments, the fortification is accomplished by spraying a liquid solution onto the biomass (or other consumer product). In several embodiments, by drying to completeness, a product that is fortified with an active is provided. In several embodiments, these fortifying therapeutic agents can be used to enhance health benefits of the consumer product (e.g., biomass), to change the flavor profile of the consumer product (e.g., biomass), to change the physiological effects of the consumer product (e.g., biomass), and/or to provide other benefits.

In several embodiments, where a topical is provided, the topical formulation may include SLM2026™ (skin lipid matrix including Aqua (Water), Caprylic/Capric Triglyceride, Hydrogenated Phosphatidylcholine, Pentylene Glycol, Glycerin, Butyrospermum Parkii (Shea) Butter, Squalane, Ceramide NP), SLM2038™ (skin lipid matrix including Aqua (and) Caprylic/Capric Triglyceride (and) Hydrogenated Phosphatidylcholine (and) Pentylene Glycol (and) Glycerin (and) Butyrospermum Parkii Butter (and) Squalane), or other formulated emulsion systems. In several embodiments, where a topical is provided, topical permeation enhancers may be included and may be selected from, but not inclusive of, the following: dimethyl sulfoxide, dimethyl sulfone, ethanol, propylene glycol, dimethyl isosorbide, polyvinyl alcohol, Capryol™ 90™, Labrafil M1944 CS,™ Labrasol™, Labrasol ALF™, LauroglycolT M90™, Transcutol HP™, Capmul S12L™, Campul PG-23 EP/NF™, Campul PG-8 NF™. The topical may include one or more of Lipoid's Skin Lipid Matrix 2026™ technology, lipid/oil based ingredients or oil soluble ingredients, and includes Captex 170 EP™ as a skin permeation enhancer, argan oil, menthol, arnica oil, camphor, grapefruit seed oil, For example, dimethyl sulfoxide, dimethyl isosorbide, topical analgesics such as lidocaine, wintergreen oil, and terpenes such as guaiacol. In several embodiments, any one or more of these ingredients is present in the topical composition at a dry wt. % of equal to or less than about: 5%, 10%, 15%, 20%, 30%, 40%, 50%, 60%, or ranges including and/or spanning the aforementioned values. In several embodiments, any one or more of these ingredients is present in the topical at a wet wt. % of equal to or at least about: 2.5%, 5%, 7.5%, 10%, 12.5%, 15%, 20%, 30%, or ranges including and/or spanning the aforementioned values.

In several embodiments, the nanoparticle compositions disclosed herein can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). In several embodiments, these additional agents are not added. Such preparations can include liposomes, microemulsions, micelles, and/or unilamellar or multilamellar vesicles.

In several embodiments, the nanoparticle composition is configured for oral ingestion. In several embodiments, the nanoparticle formulation is provided as a drinkable solution, such as a beverage, elixir, tonic, or the like. In several embodiments, the nanoparticle formulation is provided as a powder that can be constituted in a liquid (e.g., water, juice, coffee, beer, soda) and ingested orally.

In some embodiments, the composition particle, when added to a second composition (e.g., a beverage), maintains a turbidity (EBC) of less than about 1, 2, 3, 4, or 5 EBC over about 1, 2, 3, 4, 5, 6, 7, 8, 9, 10, or greater than 10 days at about 37° C., 38° C., 39° C., and/or 40° C., with about 50%, 55%, 60%, 65%, 70%, 75%, 80%, 85%, 90%, 95%, or 100% relative humidity. In some embodiments, the composition particle, when added to a second composition (e.g., a beverage), maintains a turbidity (EBC) of less than about 4 EBC over at least 5 days at about 40° C. with about 75%, relative humidity. In some embodiments, the composition particle, when added to a second composition (e.g., a beverage), maintains a turbidity (EBC) of less than about 2.5 EBC over at least 5 days at about 40° C. with about 75%, relative humidity. In some embodiments, the composition particle, when added to a second composition (e.g., a beverage), maintains a turbidity (EBC) of less than about 1 EBC over at least 5 days at about 40° C. with about 75%, relative humidity. In some embodiments, the composition particle, when added to a second composition (e.g., a beverage), maintains a turbidity (EBC) that does not significantly differ over at least 5 days at about 40° C. with about 75%, relative humidity. In some embodiments, the composition particle, when added to a second composition (e.g., a beverage), maintains a turbidity (EBC) that does not significantly differ from a control composition (e.g., a beverage) that does not comprise the aqueous composition over at least 5 days at about 40° C. with about 75%, relative humidity.

For administration (e.g., oral), the nanoparticle compositions can be provided as a tablet, capsule, pressed tablet, aqueous or oil suspension, dispersible powder or granule (as a food additive, drink additive, etc.), emulsion, hard or soft capsule, syrup or elixir. Compositions intended for oral use can include one or more of the following agents: sweeteners, flavoring agents, coloring agents and preservatives. In several embodiments, the compositions are provided in ready-to-drink formulations, such as protein drinks, energy drinks, sodas, juices, coffees, etc.

Formulations for oral use can also be provided as gelatin capsules. In several embodiments, a powder composition as disclosed herein is added to the gelatin capsule. In several embodiments, the active ingredient(s) in the nanoparticle compositions disclosed herein are mixed with an inert solid diluent, such as calcium carbonate, calcium phosphate, or kaolin, or as soft gelatin capsules. In soft capsules, the active compounds can be dissolved or suspended in suitable liquids, such as water. Stabilizers and microspheres formulated for oral administration can also be used. Capsules can include push-fit capsules made of gelatin, as well as soft, sealed capsules made of gelatin and a plasticizer, such as glycerol or sorbitol.

In capsule formulations, trehalose can be added. In several embodiments, trehalose is present in the nanoparticle composition at a dry wt. % of equal to or less than about: 5%, 10%, 15%, 20%, 30%, 40%, 50%, 60%, or ranges including and/or spanning the aforementioned values. In several embodiments, the trehalose is present in the composition at a wet wt. % of equal to or at least about: 2.5%, 5%, 7.5%, 10%, 12.5%, 15%, 20%, 30%, or ranges including and/or spanning the aforementioned values.

### Methods

Dry powder formulations or liquid embodiments may also be used in a variety of consumer products. For example, in some embodiments, dry powders can be added (e.g., scooped, from a packet, squirted from a dispenser, etc.) into any consumer product (e.g., a hot or cold beverage).

In several embodiments, liquid solutions or powdered nanoparticle formulations can be coated onto and/or added into a consumer product (e.g., sprayed and/or squirted from a dispenser, through dipping, soaking, rolling, dusting, etc.). In several embodiments, the consumer product is a food product (e.g., candies, lollipops, edibles, food, ingestible, buccal adhesives, or others). In several embodiments, the consumer product is a biomass. In several embodiments, the nanoparticles (e.g., of the compositions disclosed herein) supplement and/or fortify the consumer product (e.g., biomass) with an active agent from the nanoparticles. In several embodiments, the active agent is delivered to the user in a greater quantity than would be achieved using (e.g., consuming) the biomass alone.

In several embodiments, the nanoparticle compositions may be used to improve a condition. In several embodiments, an improvement in a condition can be a reduction in disease symptoms or manifestations (e.g., opioid withdrawal symptoms, pain, anxiety & stress, mood disorders (e.g., depression), seizures, malaise, inflammation, insomnia, etc.). Actual dosage levels of active ingredients in an active composition of the presently disclosed subject matter can be varied so as to administer an amount of the active compound(s) that is effective to achieve the desired response for a particular subject and/or application. The selected dosage level will depend upon a variety of factors including, but not limited to, the activity of the composition, composition, route of administration, combination with other drugs or treatments, severity of the condition being treated, and the physical condition and prior medical history of the subject being treated. In several embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

In some embodiments, the compositions disclosed herein can be used in methods of treatment and can be administered to a subject having a condition to be treated. In some embodiments, the subject is treated by administering an effective amount of a composition (e.g., lipid-based particle compositions) as disclosed herein to the subject.

In some embodiments, the disease or condition to be treated via administration of a composition as disclosed herein may include one or more of liver protection, hangover prevention, vitamin deficiency, alcohol related heart disease, alcohol related neurological conditions, opioid withdraw, attention deficient disorder (ADHD), pain, anxiety, depression, seizures, malaise, nausea, insomnia, work-sleep shift disorder, sleep disturbances, inflammation, immunity, epilepsy, diabetes, cancer (breast, colon, prostate, glioma, etc.), etc.

In some embodiments, the lipid-based particle composition is provided for use in a method of treating a subject suffering from a condition selected from pain associated disorders, inflammatory disorders and conditions, symptoms of vomiting and nausea, intestine and bowl disorders, disorders and conditions associated with anxiety, disorders and conditions associated with vitamin deficiency, disorders and conditions associated with psychosis, disorders and conditions associated with seizures and/or convulsions, sleep disorders and conditions, disorders and conditions which require treatment by immunosuppression, disorders and conditions associated with elevated blood glucose levels, disorders and conditions associated with nerve system degradation, inflammatory skin disorders and conditions, disorders and conditions associated with artery blockage, disorders and conditions associated with bacterial infections, disorders and conditions associated with fungal infections, proliferative disorders and conditions, and disorders and conditions associated with inhibited bone growth, post trauma disorders and others, a patient in need of appetite suppression or stimulation. In some embodiments, the method comprises administering to the subject an effective amount of a composition of this disclosure.

In several embodiments, surprisingly, an aqueous nanoparticle composition comprising an active agent (e.g., pharmaceuticals, nutraceuticals, cosmetics, pigments, flavorings, and the like) as disclosed herein may be administered using an atomizer. In several embodiments, an atomizer nozzles are used in oral spray, such as the Binaca Spray™. In several embodiments, an atomizer nozzle is used in a nasal spray. This result is surprising, as some extracts disclosed herein would be typically be understood to clog atomizer nozzles.

In several embodiments, as disclosed elsewhere herein, the nanoparticles or compositions may be used as coatings. In several embodiments, coating is performed with an aqueous or solvent solution of the nanoparticles. For example, the solution may be sprayed (e.g., via a spray nozzle, atomizer, etc.) or otherwise coated (e.g., dip-coated, etc.). In several embodiments, pharmaceutical coating equipment (e.g., that used to coat tablets, beads, drug layered/coated films) is used to coat the biomass. In several embodiments, fluid bed technology, film bed technology, dry powder laying technology, and/or combinations thereof are used to coat the biomass. In several embodiments, film coating is used.

In several embodiments, prior to coating with a liquid solution of nanoparticles, the material to be coated is dried completely. Then, after coating, the coated material may be dried. In other implementations, the material to be coated is not dried before being coated. In several embodiments, as disclosed elsewhere herein, a powder can be used to coat the material to be coated. In several embodiments, a powder nanoparticle formulation is dusted or coated onto the material to be coated. Additional drying may be performed to afford a consumable fortified product. In several embodiments, where the material to be coated is dried prior to coating with a powder nanoparticle, an additional drying step may optionally be performed (though it may not be required). In several embodiments, the dried fortified material is suitable for use by a user.

In several embodiments, the fortified material is further processed prior to use (e.g., in dried or undried form). In several embodiments, milling is used to reduce the size of the coated material particles. In several embodiments, the milling is a two stage process with a first course milling and then a fine milling. In some embodiment, after milling (dry or wet) the average particle size of the fortified material is such that greater than 50% pass through screen having a mesh size of less than or equal to 100, 150, 200, or ranges spanning and/or including the aforementioned values. In some embodiment, after milling (dry or wet) the average particle size of the fortified material is less than or equal to about: 1000 μm, 500 μm, 200 μm, or ranges including and/or spanning the aforementioned values. In several embodiments, after drying and/or milling, the fortified material is suitable for delivery to a user.

In several embodiments, the nanoparticle formulation, can be remote loaded with active agents. In several embodiments, a liquid formulation of nanoparticles is adding to an active agent. In several embodiments, the active agent incorporates into the particles by hydrophobic/hydrophilic interactions, electrostatic interactions, etc. In several embodiments, a remote loaded product could be coated onto biomass (as disclosed above), dried, and/or milled to provide a fortified, finished product. In several embodiments, the nanoparticle can be provided with or without an active agent inside prior to remote loading. Then, other active agents can be loaded into that particle through remote loading.

In several embodiments, liquid formulations can be added measured and poured into any consumer product. In several embodiments, the consumer product can include one or more alcoholic beverages, milks (dairy, but also nuts “milks” such as almond juice, etc.), coffee, sodas, tea, fermented beverages, wines, nutritional supplements, smoothies, simple water, sports drinks, sparkling water, or the like. In several embodiments, the consumer product can include one or more eye drops, mouth wash, lotions/creams/serums, lip balms, hair care products, deodorant, nasal solutions, enema solutions, liquid soaps, solid soaps, or the like. In several embodiments, the consumer product can include one or more food products. In several embodiments, the consumer product can include desserts. In several embodiments, the consumer product can include single serving products of multi-serving products (e.g., family size). In several embodiments, the consumer product can include one or more dried products (e.g., flour, coffee creamer, protein shakes, nutritional supplements, etc.). In several embodiments, these dried products can be configured to be reconstituted for use. In several embodiments, the consumer product can include one or more the dried product can be added to other dietary supplements (e.g., multivitamins, gummies, etc.).

The agents in some aspects of the disclosure may be formulated into preparations for local delivery (such as to a specific location of the body, such as a specific tissue or cell type) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain aspects, the pharmaceutical compositions are administered in the form of injectable compositions either as liquid solutions or suspensions; solid forms suitable or solution in, or suspension in, liquid prior to injection may also be prepared. These preparations also may be emulsified. In some embodiments, the composition comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. In further aspects, the pharmaceutical compositions may include classic pharmaceutical preparations.

Administration of compositions according to certain aspects may be via any common route so long as the target tissue is available via that route. This may include oral, nasal, buccal, rectal, vaginal or topical administration. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular active substance. It is contemplated that other agents may be used in combination with certain aspects of the present embodiments to improve the therapeutic efficacy of treatment.

Surprisingly and advantageously, some embodiments disclosed herein do not require one or more ingredients typically used to prepare liposomes and/or nanoparticle formulations. In some embodiments, the lipid-based particle compositions disclosed herein lack, contain less than about 2%, less than about 0.5%, and/or substantially none of one or more of lipids (other than phospholipids), triglycerides, sterols, lecithin surfactants, hyaluronic acid, Alcolec S, Alcolec BS, Alcolec XTRA-A, polysorbates (such as Polysorbate 80 and Polysorbate 20), monoglycerides, diglycerides, glyceryl oleate, poloxamers, terpenes, sodium alginate, polyvinylpyrrolidone, L-alginate, chondroitin, poly gamma glutamic acid, gelatin, chitosan, corn starch, polyoxyl 40-hydroxy castor oil, Tween 20, Span 80, or the salts of any of thereof. In some embodiments, the lipid-based particle compositions disclosed herein lack, contain less than about 2%, less than about 0.5%, and/or substantially none of a surfactant and/or emulsifiers other than a phospholipid. In some embodiments, the lipid-based particle compositions disclosed herein lack an emulsifier at concentrations greater than about 1/10^{th} that of a phospholipid. In some embodiments, the lipid-based particle compositions disclosed herein lack an emulsifier at concentrations greater than about 1/10^{th} that of a phosphatidylcholine. In some embodiments, the lipid-based particle compositions lack unhydrogenated phospholipids. In some embodiments, the lipid-based particle compositions lack hydrogenated phospholipids. In some embodiments, the lipid-based particle compositions comprise one or more unhydrogenated or hydrogenated phospholipids. In some embodiments, the lipid-based particle compositions disclosed herein lack, contain less than about 2%, and/or less than about 0.5% of one or more of a buffering agent, a polymeric stabilizing agent, and/or sodium hydroxide.

In some embodiments, the lipid-based particle compositions disclosed herein lack nonnatural ingredients. In some embodiments, the lipid-based particle compositions disclosed are synthetic and not found in nature.

In some embodiments, the lipid-based particle compositions disclosed herein lack, contain less than about 2%, less than about 0.5%, and/or substantially none of one or more organic bases (which may include, but are not limited to: butyl hydroxyl anisole (BHA), butyl hydroxyl toluene (BHT) and sodium ascorbate). In some embodiments, the lipid-based particle compositions disclosed herein lack, contain less than about 2%, less than about 0.5%, and/or substantially none of whey protein isolate. In some embodiments, the lipid-based particle compositions disclosed herein lack, contain less than about 2%, less than about 0.5%, and/or substantially none of ticamulsion 3020, purity gum, gum Arabic, and/or a modified gum Arabic. In some embodiments, the lipid-based particle compositions disclosed herein lack, contain less than about 2%, less than about 0.5%, and/or substantially none one or more of fatty acids, triglycerides triacylglycerols, acylglycerols, fats, waxes, sphingolipids, glycerides, sterides, cerides, glycolipids, sulfolipids, lipoproteins, chylomicrons and the derivatives of these lipids. In some embodiments, the lipid-based particle compositions disclosed herein lack, contain less than about 2%, less than about 0.5%, and/or substantially none of a surfactant. In some embodiments, the lipid-based particle compositions disclosed herein lack, contain less than about 2%, less than about 0.5%, and/or substantially none of one or more of polyglycolized glycerides and polyoxyethylene glycerides of medium to long chain mono-, di-, and triglycerides, such as: almond oil PEG-6 esters, almond oil PEG-60 esters, apricot kernel oil PEG-6 esters (Labrafil® M1944CS), caprylic/capric triglycerides PEG-4 esters (Labrafac® Hydro WL 1219), caprylic/capric triglycerides PEG-4 complex (Labrafac® Hydrophile), caprylic/capric glycerides PEG-6 esters (Softigen® 767), caprylic/capric glycerides PEG-8 esters (Labrasol®), castor oil PEG-50 esters, hydrogenated castor oil PEG-5 esters, hydrogenated castor oil PEG-7 esters, 9 hydrogenated castor oil PEG-9 esters, corn oil PEG-6 esters (Labrafil® M 2125 CS), corn oil PEG-8 esters (Labrafil® WL 2609 BS), corn glycerides PEG-60 esters, olive oil PEG-6 esters (Labrafil® M1980 CS), hydrogenated palm/palm kernel oil PEG-6 esters (Labrafil® M 2130 BS), hydrogenated palm/palm kernel oil PEG-6 esters with palm kernel oil, PEG-6, palm oil (Labrafil® M 2130 CS), palm kernel oil PEG-40 esters, peanut oil PEG-6 esters (Labrafil® M 1969 CS), glyceryl laurate/PEG-32 laurate (Gelucire® 44/14), glyceryl laurate glyccry I/PEG 20 laurate, glyceryl laurate glyceryl/PEG 32 laurate, glyceryl, laurate glyceryl/PEG 40 laurate, glyceryl oleate/PEG-20 glyceryl, glyceryl oleate/PEG-30 oleate, glyceryl palmitostearate/PEG-32 palmitostearate (Gelucire® 50/13), glyceryl stearate/PEG stearate, glyceryl stearate/PEG-32 stearate (Gelucire® 53/10), saturated polyglycolized glycerides (Gelucire® 37/02 and Gelucire® 50/02), triisostearin PEG-6 esters (i.e. Labrafil® Isostearique), triolein PEG-6 esters, trioleate PEG-25 esters, polyoxyl 35 castor oil (Cremophor® EL or Kolliphor® EL), polyoxyl 40 hydrogenated castor oil (Cremophor® RH 40 or Kolliphor® RH40), polyoxyl 60 hydrogenated castor oil (Cremophor® RH60), polyglycolized derivatives and polyoxyethylene esters or ethers derivatives of medium to long chain fatty acids, propylene glycol esters of medium to long chain fatty acids, which can be used including caprylate/caprate diglycerides, glyceryl monooleate, glyceryl ricinoleate, glyceryl laurate, glyceryl dilaurate, glyceryl dioleate, glyceryl mono/dioleate, polyglyceryl-10 trioleate, poly glyceryl-10 laurate, polyglyceryl-10 oleate, and poly glyceryl-10 mono dioleate, propylene glycol caprylate/caprate (Labrafac® PC), propylene glycol dicaprylate/dicaprate (Miglyol® 840), propylene glycol monolaurate, propylene glycol ricinoleate, propylene glycol monooleate, propylene glycol dicaprylate/dicaprate, propylene glycol dioctanoate, sucrose esters surfactants such as sucrose stearate, sucrose distearate, sucrose palmitate, sucrose oleate, and combinations thereof.

In some embodiments the lipid-based particle suspension solution disclosed herein have improved shelf life as indicated in part by the ability of the lipid-based particle composition to remain suspended in the liquid broth without aggregating or separating/settling from solution. For example, in some embodiments, the lipid-based particle suspension solution disclosed remain solubilized in pure water at room temperature, neutral pH, and atmospheric pressure for a period greater than or equal to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 24 months, or ranges including and/or spanning the aforementioned values. In some embodiments, the shelf life can be determined as the period of time in which there is 95% confidence that at least 50% of the response (therapeutic agent(s) concentration or particle size) is within the specification limit. This refers to a 95% confidence interval and when linear regression predicts that at least 50% of the response is within the set specification limit.

In some embodiments, the shelf life can be determined as a time where the concentration of the active ingredient has changed (e.g., lessened) by less than or equal to 30%, 25%, 20%, 15%, 10%, 5%, 2.5%, or ranges including and or spanning the aforementioned ranges.

In some embodiments, the compositions disclosed herein, when provided in a lipid-based particle suspension solution, have improved thermal stability.

In some embodiments, thermal sterilization includes exposing and/or heating a composition to a temperature of equal to or at least about 40° C., 50° C., 60° C., 70° C., 80° C., 90° C., 100° C., 110° C., 120° C., 130° C., 140° C., 150° C., or 160° C. In some embodiments, the thermal sterilization is performed for a time period of equal to or at least about: 1 second, 2 seconds, 3 seconds, 4 seconds, 5 seconds, 10 seconds, 15 seconds, 20 seconds, 25 seconds, 30 seconds, 45 seconds, 1 minute, 2 minutes, 3 minutes, 5 minutes, 10 minutes, 15 minutes, 30 minutes, 60 minutes, or ranges including and/or spanning the aforementioned values.

In some embodiments, after a 15 or 30 day period, the particle size and/or PDI varies by less than or equal to about: 1%, 5%, 10%, 20%, 30%, 40%, 50%, 75%, 100%, 125%, 150%, 175%, 200%, 250%, 300%, or ranges including and/or spanning the aforementioned values. In some embodiments, using the lipid-based particle compositions disclosed herein, after a storage period of equal to or at least about 15 days, 30 days, 45 days, 60 days, 6 months, 12 months, 18 months, or 24 month periods, the concentration of the therapeutic agent in the aqueous product (e.g., a beverage, carbonated or not, alcoholic or not) drops by less than or equal to about: 0.25%, 0.5%, 0.75%, 1%, 1.5%, 2.0%, 2.5%, 5%, 7.5%, 10%, 15%, 20%, 25%, 30%, or ranges including and/or spanning the aforementioned values. In some embodiments, the storage conditions include temperatures of equal to or at least about: 2° C., 4° C., 6° C., 8° C., 10° C., 20° C., 40° C., 60° C., or ranges including and/or spanning the aforementioned values.

In some embodiments, the compositions disclosed herein, when provided in a lipid-based particle suspension solution, are stable during ozonation sterilization, UV sterilization, heat sterilization (e.g., pasteurization), filtration sterilization, and/or gamma irradiation during beverage preparation and packaging. In some embodiments, the particle size and/or PDI after sterilization (e.g., exposure to techniques that allow sterilization of the composition) varies by less than or equal to about: 1%, 5%, 10%, 20%, 30%, 40%, 50%, 60%, 75%, 100%, 125%, 150%, 175%, 200%, 250%, 300%, or ranges including and/or spanning the aforementioned values. In some embodiments, the therapeutic agent concentration after sterilization (e.g., exposure to techniques that allow sterilization of the composition) drops by less than or equal to about: 0.05%, 0.1%, 0.5%, 1%, 5%, 10%, 15%, 20%, 25%, 30%, or ranges including and/or spanning the aforementioned values. In some embodiments, after sterilization, the lipid-based particle suspension solutions comprising lipid-based particle compositions have a shelf life of equal to or greater than 6 months, 12 months, 14 months, 16 months, 18 months, 19 months, 24 months, or ranges including and/or spanning the aforementioned values (e.g., a standard storage conditions).

Several illustrative embodiments of compositions and methods have been disclosed. Although this disclosure has been described in terms of certain illustrative embodiments and uses, other embodiments and other uses, including embodiments and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various embodiments. All possible combinations and subcombinations of elements and components described herein are intended to be included in this disclosure. No single feature or group of features is necessary or indispensable.

Certain features that are described in this disclosure in the context of separate implementations can also be implemented in combination in a single implementation. Conversely, various features that are described in the context of a single implementation also can be implemented in multiple implementations separately or in any suitable subcombination. Moreover, although features may be described above as acting in certain combinations, one or more features from a claimed combination can in some cases be excised from the combination, and the combination may be claimed as a subcombination or variation of a subcombination.

Any portion of any of the steps, processes, and/or compositions disclosed or illustrated in one embodiment, flowchart, or example in this disclosure can be combined or used with (or instead of) any other portion of any of the steps, processes, and/or compositions disclosed or illustrated in a different embodiment, flowchart, or example. The embodiments and examples described herein are not intended to be discrete and separate from each other. Combinations, variations, and other implementations of the disclosed features are within the scope of this disclosure.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### I. Example 1: Preparation of Embodiments of the Composition

Unless otherwise noted, the phosphatidylcholine was sunflower derived and included over 20% phosphatidylcholine (PS20).

Particle size and zeta potential of liquid was measured on a Malvern ZS90 Zetasizer (Malvern, UK). Products were measured in low-volume, disposable cuvettes and zeta cassettes. Active agent concentrations, related substances and identity (retention time) were measure by high-pressure liquid chromatography (HPLC) in house, by Eurofins, or by Certified Labs (formerly MQL). Residual solvents were measured by gas chromatography (GC), and heavy metals by inductive coupled plasma-optical emission spectrometry (ICP-OES) in house or at 374 Labs (Reno, NV). Rapid preservative effectiveness testing can be determined by a reduction in colony forming units (CFU) of test microorganisms at Microchem Laboratory (Round Rock, Texas). Preservative effectiveness testing can confirmed that the compositions were resistant to bacterial growth (by measuring colony forming units (CFUs) per volume in a given amount of time.

Manufacturing Process: Active agent containing- and non-active agent containing-lipid nanoparticles were prepared using a solvent-free manufacturing process, as shown in Table 1. To prepare the mixed micelle composition, water was heated to 75° C. before adding additional ingredients. The pH was adjusted. The required amounts of saponins were dispersed in the aqueous solution at varying concentrations, as well as other hydrophilic components. Afterwards, the active agent as well as additional lipophilic components were added with high shear mixing to achieve a final active concentration of 10 mg/mL. The liquid was high shear mixed until a transparent solution resulted. After sufficient mixing, it was passed through a 0.45 μm PES filter.

### II. Example 2: Preparation of Nanoparticles and Stability Study

Multiple nanoparticles were prepared using the manufacturing process described in Example 1. 250 gram batches containing the concentrations of components described in Table 4 below were prepared and 30 grams were filled into 30 mL dropper cap bottles for stability testing. Samples were stored at controlled 2-8° C., room temperature (25° C./60% relative humidity), or accelerated conditions (40° C./75% relative humidity) for testing. Shown in Table 2 are changes in concentration of the active agent, changes in Z-average particle size, and changes in Polydispersity Index (PDI) over the period of time tested for each storage condition.

Storage at 40° C./75% is generally understood to increase degradation kinetics by a factor of 4 compared to controlled room temperature based on the Arrhenius equation.

It was found that the higher the concentration of the plant extract the lower the D90 of the formulations with or without microfluidization. Z-avg particle size without microfluidization was also decreased with higher concentrations of the plant extract. It was also found that the lower the concentration of the phosphatidylcholine or plant extract or oil or excipients (e.g., vitamin E) the lower the viscosity of the formulations. Further, it is seen that the formulations with the plant extract showed little to no phase separation under accelerated conditions (e.g., storage at 40° C. and higher).

TABLE 4

| | Percent of ingredients | | | | | | | | |
|---|---|---|---|---|---|---|---|---|---|
| | Saponin source | Sodium | Potassium | Sodium | Vitamin | | | | |
| Formulation | and concentration | Benzoate | Sorbate | Ascorbate | D3 | Vitamin E | Olive Oil | PS20 | Water |
| Formulation 1 - | Camellia sinesis | 0.1 | 0.1 | 0.2 | 0.1 | 0 | 33.6 | 10 | 53.39 |
| Vitamin D3 | extract, 0% | | | | | | | | |
| Formulation 2 - | Camellia sinesis | 0.1 | 0.1 | 0.2 | 0.1 | 1.5 | 33.6 | 9 | 53.39 |
| Vitamin D3 | extract, 0.1% | | | | | | | | |
| Formulation 3 - | Camellia sinesis | 0.1 | 0.1 | 0.2 | 0.1 | 1.5 | 33.6 | 10 | 53.39 |
| Vitamin D3 | extract, 0.05% | | | | | | | | |
| Formulation 4 - | Camellia sinesis | 0.1 | 0.1 | 0.2 | 0.1 | 3 | 33.6 | 9 | 53.39 |
| Vitamin D3 | extract, 0% | | | | | | | | |
| Formulation 5 - | Camellia sinesis | 0.1 | 0.1 | 0.2 | 0.1 | 0 | 33.6 | 11 | 53.39 |
| Vitamin D3 | extract, 0.05% | | | | | | | | |
| Formulation 6 - | Camellia sinesis | 0.1 | 0.1 | 0.2 | 0.1 | 3 | 33.6 | 10 | 53.39 |
| Vitamin D3 | extract, 0.1% | | | | | | | | |
| Formulation 7 - | Camellia sinesis | 0.1 | 0.1 | 0.2 | 0.1 | 0 | 33.6 | 11 | 53.39 |
| Vitamin D3 | extract, 0.1% | | | | | | | | |
| Formulation 8 - | Camellia sinesis | 0.1 | 0.1 | 0.2 | 0.1 | 3 | 33.6 | 11 | 53.39 |
| Vitamin D3 | extract, 0.1% | | | | | | | | |
| Formulation 9 - | Camellia sinesis | 0.1 | 0.1 | 0.2 | 0.1 | 0 | 33.6 | 9 | 53.39 |
| Vitamin D3 | extract, 0% | | | | | | | | |
| Formulation 10 - | Camellia sinesis | 0.1 | 0.1 | 0.2 | 0.1 | 3 | 33.6 | 9 | 53.39 |
| Vitamin D3 | extract, 0.05% | | | | | | | | |
| Formulation 11 - | Camellia sinesis | 0.1 | 0.1 | 0.2 | 0.1 | 0 | 33.6 | 9 | 53.39 |
| Vitamin D3 | extract, 0.1% | | | | | | | | |
| Formulation 12 - | Camellia sinesis | 0.1 | 0.1 | 0.2 | 0.1 | 3 | 33.6 | 11 | 53.39 |
| Vitamin D3 | extract, 0% | | | | | | | | |
| Formulation 13 - | Camellia sinesis | 0.1 | 0.1 | 0.2 | 0.1 | 1.5 | 33.6 | 11 | 53.39 |
| Vitamin D3 | extract, 0% | | | | | | | | |

| | Percent of ingredients | | | | | | | | | |
|---|---|---|---|---|---|---|---|---|---|---|
| | Plant | Fiber | | | | Docosa- | | | | |
| | extract and | from | Ascorbic | Sodium | Squalene | hexaenoic | Vitamin | | | |
| Formulation | concentration | Mushroom | acid | ascorbate | oil | acid (DHA) | E | Olive Oil | PS20 | Water |
| Formulation 14 - | Camellia sinesis | 0.6 | 0.2 | 0.2 | 18.6 | — | 0.1 | 12 | 10 | 57.3 |
| Squalene Oil | extract, 1% | | | | | | | | | |
| Formulation 15 - | Camellia sinesis | 0.6 | 0.2 | 0.2 | — | 1 | 2 | 27 | 10 | 56.24 |
| DHA | extract, 1% | | | | | | | | | |

TABLE 5

| Sample and | | | | | | | | | | | | | | | | |
|---|---|---|---|---|---|---|---|---|---|---|---|---|---|---|---|---|
| Viscosity at | Micro- | | Potency (mg/g) | | | | Particle size (z-avg, nm) | | | | | Particle size (D90, nm) | | | | |
| T0 on Pass | fluidizer | Temp | | 1 | 2 | 3 | | 1 | 2 | 3 | 4 | | 1 | 2 | 3 | 4 |
| 0 (mPa*s) | Passes | (° C.) | Initial | month | mo. | mo. | Initial | month | mo. | mo. | mo. | Initial | month | mo. | mo. | mo. |
| Formulation | 0 | 25 | 0.939 | | | | 529.9 | | | | | 9510 | | | | |
| 1, | | 40 | | | | | | | | | | | | | | |
| 164.375 | | 55 | | 0.8980 | | | | 643.4 | | | | | 9080 | | | |
| | 1 | 25 | 0.872 | | | | 292.1 | | | | | 6320 | | | | |
| | | 40 | | | | | | | | | | | | | | |
| | | 55 | | 0.811 | | | | 321.5 | | | 329.4 | | 1420 | | | 1080 |
| | 2 | 25 | 0.876 | | 0.0000 | 0.0000 | 286.9 | | | | | 5290 | | | | |
| | | 40 | | | | | | | | | | | | | | |
| | | 55 | | 0.817 | | | | 320.1 | | | 335.7 | | 752 | | | 824 |
| Formulation | 0 | 25 | 0.914 | | | | 543.8 | | | | | 9150 | | | | |
| 2, | | 40 | | | | | | | | | | | | | | |
| 56.275 | | 55 | | 0.732 | | | | 499.9 | | | | | 8730 | | | |
| | 1 | 25 | 0.854 | | | | 302 | | | | | 6630 | | | | |
| | | 40 | | | | | | | | | | | | | | |
| | | 55 | | 0.787 | | | | 310.9 | | | 310.7 | | 782 | | | 726 |
| | 2 | 25 | 0.873 | | | | 296.4 | | | | | 806 | | | | |
| | | 40 | | | | | | | | | | | | | | |
| | | 55 | | 0.834 | | | | 309.4 | | | 317.5 | | 589 | | | 817 |
| Formulation | 0 | 25 | 0.96 | | | | 852.2 | | | | | 7100 | | | | |
| 3, | | 40 | | | | | | | | | | | | | | |
| 179.55 | | 55 | | | | | | 652.5 | | | | | 7190 | | | |
| | 1 | 25 | 0.845 | | | | 301.2 | | | | | 690 | | | | |
| | | 40 | | | | | | | | | | | | | | |
| | | 55 | | | | | | 306.7 | | | 323.1 | | 1030 | | | 912 |
| | 2 | 25 | 0.862 | | | | 289.4 | | | | | 673 | | | | |
| | | 40 | | | | | | | | | | | | | | |
| | | 55 | | | | | | 314 | | | 327.3 | | 820 | | | 1030 |
| Formulation | 0 | 25 | 1.021 | | | | 486.3 | | | | | 8540 | | | | |
| 4, | | 40 | | | | | | | | | | | | | | |
| 56.7 | | 55 | | | | | | 347.6 | | | | | 9180 | | | |
| | 1 | 25 | 0.936 | | | | 305.2 | | | | | 801 | | | | |
| | | 40 | | | | | | | | | | | | | | |
| | | 55 | | | | | | 316.3 | | | 319 | | 852 | | | 755 |
| | 2 | 25 | 0.966 | | | | 291 | | | | | 4860 | | | | |
| | | 40 | | | | | | | | | | | | | | |
| | | 55 | | | | | | 294.9 | | | 315.9 | | 812 | | | 658 |
| Formulation | 0 | 25 | 0.964 | | | | 714.8 | | | | | 8610 | | | | |
| 5, | | 40 | | | | | | | | | | | | | | |
| 3731 | | 55 | | 0.9 | | | | 580.9 | | | 593.1 | | 9550 | | | 9600 |
| | 1 | 25 | 0.742 | | | | 251.7 | | | | | 5390 | | | | |
| | | 40 | | | | | | | | | | | | | | |
| | | 55 | | 0.737 | | | | 278 | | | 289.5 | | 897 | | | 724 |
| | 2 | 25 | 0.887 | | | | 249.3 | | | | | 516 | | | | |
| | | 40 | | | | | | | | | | | | | | |
| | | 55 | | 0.826 | | | | 299.7 | | | 310.4 | | 518 | | | 731 |
| Formulation | 0 | 25 | 0.851 | | | | 740.2 | | | | | 9500 | | | | |
| 6, | | 40 | | | | | | | | | | | | | | |
| 237.5333 | | 55 | | | | | | 587.6 | | | 398.5 | | 9550 | | | 9280 |
| | 1 | 25 | 0.684 | | | | 284.4 | | | | | 760 | | | | |
| | | 40 | | | | | | | | | | | | | | |
| | | 55 | | | | | | 296.8 | | | 314.2 | | 817 | | | 554 |
| | 2 | 25 | 0.781 | | | | 268.6 | | | | | 692 | | | | |
| | | 40 | | | | | | | | | | | | | | |
| | | 55 | | | | | | 305.6 | | | 325.8 | | 702 | | | 550 |
| Formulation | 0 | 25 | 1.093 | | | | 383.9 | | | | | 9230 | | | | |
| 7, | | 40 | | | | | | | | | | | | | | |
| 840.7 | | 55 | | | | | | 498.3 | | | 519.2 | | 9130 | | | 9540 |
| | 1 | 25 | 0.999 | | | | 271.4 | | | | | 419 | | | | |
| | | 40 | | | | | | | | | | | | | | |
| | | 55 | | | | | | 322 | | | 353.9 | | 1340 | | | 594 |
| | 2 | 25 | 0.987 | | | | 268.9 | | | | | 799 | | | | |
| | | 40 | | | | | | | | | | | | | | |
| | | 55 | | | | | | 320.9 | | | 344.7 | | 694 | | | 867 |
| Formulation | 0 | 25 | 0.862 | | | | 659.7 | | | | | 9260 | | | | |
| 8, | | 40 | | | | | | | | | | | | | | |
| 17496.67 | | 55 | | 1.03 | | | | 394.1 | | | 553.6 | | 3750 | | | 9470 |
| | 1 | 25 | 0.75 | | | | 275.9 | | | | | 703 | | | | |
| | | 40 | | | | | | | | | | | | | | |
| | | 55 | | 0.869 | | | | 315.2 | | | 332 | | 746 | | | 689 |
| | 2 | 25 | 0.75 | | | | 258.4 | | | | | 826 | | | | |
| | | 40 | | | | | | | | | | | | | | |
| | | 55 | | 0.829 | | | | 322.6 | | | 335.1 | | 700 | | | 811 |
| Formulation | 0 | 25 | 0.711 | | | | 657.9 | | | | | 9070 | | | | |
| 9, | | 40 | | | | | | | | | | | | | | |
| 48.1333 | | 55 | | 0.675 | | | | 434.2 | | | | | 9520 | | | |
| | 1 | 25 | 0.642 | | | | 306.3 | | | | | 7120 | | | | |
| | | 40 | | | | | | | | | | | | | | |
| | | 55 | | 0.715 | | | | 306.6 | | | 318.8 | | 1010 | | | 970 |
| | 2 | 25 | 0.676 | | | | 303.8 | | | | | 821 | | | | |
| | | 40 | | | | | | | | | | | | | | |
| | | 55 | | 0.78 | | | | 313.8 | | | 329.6 | | 902 | | | 696 |
| Formulation | 0 | 25 | 0.781 | | | | 576 | | | | | 8930 | | | | |
| 10, | | 40 | | | | | | | | | | | | | | |
| 46.4667 | | 55 | | 0.894 | | | | 475.4 | | | | | 8910 | | | |
| | 1 | 25 | 0.609 | | | | 314.3 | | | | | 5460 | | | | |
| | | 40 | | | | | | | | | | | | | | |
| | | 55 | | 0.782 | | | | 309.7 | | | 305 | | 758 | | | 606 |
| | 2 | 25 | 0.745 | | | | 298.2 | | | | | 829 | | | | |
| | | 40 | | | | | | | | | | | | | | |
| | | 55 | | 0.844 | | | | 297.1 | | | 310.5 | | 969 | | | 785 |
| Formulation | 0 | 25 | 0.774 | | | | 297.2 | | | | | 974 | | | | |
| 11, | | 40 | | | | | | | | | | | | | | |
| 48.9667 | | 55 | | 0.927 | | | | 461.6 | | | | | 8700 | | | |
| | 1 | 25 | 0.745 | | | | 376.5 | | | | | 457 | | | | |
| | | 40 | | | | | | | | | | | | | | |
| | | 55 | | 0.844 | | | | 310.7 | | | 301.5 | | 1330 | | | 4760 |
| | 2 | 25 | 0.745 | | | | 310.9 | | | | | 706 | | | | |
| | | 40 | | | | | | | | | | | | | | |
| | | 55 | | 0.801 | | | | 324.2 | | | 336.5 | | 962 | | | 803 |
| Formulation | 0 | 25 | 0.777 | | | | 700.2 | | | | | 8680 | | | | |
| 12, | | 40 | | | | | | | | | | | | | | |
| 5096.667 | | 55 | | 1.022 | | | | 718 | | | 453 | | 9070 | | | 9510 |
| | 1 | 25 | 0.714 | | | | 277.6 | | | | | 771 | | | | |
| | | 40 | | | | | | | | | | | | | | |
| | | 55 | | 0.833 | | | | 308.8 | | | 320.9 | | 4680 | | | 1080 |
| | 2 | 25 | 0.769 | | | | 257.1 | | | | | 684 | | | | |
| | | 40 | | | | | | | | | | | | | | |
| | | 55 | | 0.891 | | | | 311.9 | | | 333 | | 804 | | | 660 |
| Formulation | 0 | 25 | 0.731 | | | | 709.4 | | | | | 9160 | | | | |
| 13, | | 40 | | | | | | | | | | | | | | |
| 2057 | | 55 | | 0.43 | | | | 751 | | | 711.8 | | 4380 | | | 9490 |
| | 1 | 25 | 0.605 | | | | 254.5 | | | | | 704 | | | | |
| | | 40 | | | | | | | | | | | | | | |
| | | 55 | | 0.747 | | | | 303.5 | | | 316 | | 770 | | | 627 |
| | 2 | 25 | 0.687 | | | | 246.2 | | | | | 481 | | | | |
| | | 40 | | | | | | | | | | | | | | |
| | | 55 | | 0.791 | | | | 285.3 | | | 316.3 | | 592 | | | 738 |
| Formulation | — | 25 | | | | | 215.4 | | | | | 402 | | | | |
| 14 | | 40 | | | | | | | | | | | | | | |
| | | 55 | | | | | | | | | | | | | | |
| Formulation | — | 25 | | | | | 318.5 | | | | | 1260 | | | | |
| 15 | | 40 | | | | | | | | | | | | | | |
| | | 55 | | | | | | | | | | | | | | |

| | | | PDI | | | | |
|---|---|---|---|---|---|---|---|
| | Microfluidizer | Temp | | 1 | | | |
| Sample | Passes | C. | Initial | month | 2 mo. | 3 mo. | 4 mo. |
| Formulation 1 | 0 | 25 | 0.097 | | | | |
| | | 40 | | | | | |
| | | 55 | | 0.59 | | | |
| | 1 | 25 | 0.24 | | | | |
| | | 40 | | | | | |
| | | 55 | | 0.318 | | | 0.235 |
| | 2 | 25 | 0.215 | | | | |
| | | 40 | | | | | |
| | | 55 | | 0.219 | | | 0.184 |
| Formulation 2 | 0 | 25 | 0.572 | | | | |
| | | 40 | | | | | |
| | | 55 | | 0.528 | | | |
| | 1 | 25 | 0.247 | | | | |
| | | 40 | | | | | |
| | | 55 | | 0.204 | | | 0.185 |
| | 2 | 25 | 0.205 | | | | |
| | | 40 | | | | | |
| | | 55 | | 0.192 | | | 0.207 |
| Formulation 3 | 0 | 25 | 0.055 | | | | |
| | | 40 | | | | | |
| | | 55 | | 0.822 | | | |
| | 1 | 25 | 0.179 | | | | |
| | | 40 | | | | | |
| | | 55 | | 0.247 | | | 0.232 |
| | 2 | 25 | 0.171 | | | | |
| | | 40 | | | | | |
| | | 55 | | 0.222 | | | 0.228 |
| Formulation 4 | 0 | 25 | 0.344 | | | | |
| | | 40 | | | | | |
| | | 55 | | 0.088 | | | |
| | 1 | 25 | 0.192 | | | | |
| | | 40 | | | | | |
| | | 55 | | 0.27 | | | 0.187 |
| | 2 | 25 | 0.228 | | | | |
| | | 40 | | | | | |
| | | 55 | | 0.221 | | | 0.145 |
| Formulation 5 | 0 | 25 | 0.196 | | | | |
| | | 40 | | | | | |
| | | 55 | | 0.53 | | | 0.554 |
| | 1 | 25 | 0.226 | | | | |
| | | 40 | | | | | |
| | | 55 | | 0.219 | | | 0.177 |
| | 2 | 25 | 0.15 | | | | |
| | | 40 | | | | | |
| | | 55 | | 0.63 | | | 0.15 |
| Formulation 6 | 0 | 25 | 0.097 | | | | |
| | | 40 | | | | | |
| | | 55 | | 0.125 | | | 0.488 |
| | 1 | 25 | 0.205 | | | | |
| | | 40 | | | | | |
| | | 55 | | 0.225 | | | 0.138 |
| | 2 | 25 | 0.205 | | | | |
| | | 40 | | | | | |
| | | 55 | | 0.198 | | | 0.069 |
| Formulation 7 | 0 | 25 | 0.421 | | | | |
| | | 40 | | | | | |
| | | 55 | | 0.623 | | | 0.349 |
| | 1 | 25 | 0.157 | | | | |
| | | 40 | | | | | |
| | | 55 | | 0.279 | | | 0.081 |
| | 2 | 25 | 0.226 | | | | |
| | | 40 | | | | | |
| | | 55 | | 0.184 | | | 0.185 |
| Formulation 8 | 0 | 25 | 0.83 | | | | |
| | | 40 | | | | | |
| | | 55 | | 0.757 | | | 0.654 |
| | 1 | 25 | 0.193 | | | | |
| | | 40 | | | | | |
| | | 55 | | 0.174 | | | 0.132 |
| | 2 | 25 | 0.211 | | | | |
| | | 40 | | | | | |
| | | 55 | | 0.161 | | | 0.154 |
| Formulation 9 | 0 | 25 | 0.191 | | | | |
| | | 40 | | | | | |
| | | 55 | | 0.453 | | | |
| | 1 | 25 | 0.249 | | | | |
| | | 40 | | | | | |
| | | 55 | | 0.265 | | | 0.25 |
| | 2 | 25 | 0.226 | | | | |
| | | 40 | | | | | |
| | | 55 | | 0.246 | | | 0.173 |
| Formulation 10 | 0 | 25 | 0.15 | | | | |
| | | 40 | | | | | |
| | | 55 | | 0.299 | | | |
| | 1 | 25 | 0.223 | | | | |
| | | 40 | | | | | |
| | | 55 | | 0.229 | | | 0.129 |
| | 2 | 25 | 0.248 | | | | |
| | | 40 | | | | | |
| | | 55 | | 0.242 | | | 0.174 |
| Formulation 11 | 0 | 25 | 0.28 | | | | |
| | | 40 | | | | | |
| | | 55 | | 0.603 | | | |
| | 1 | 25 | 0.784 | | | | |
| | | 40 | | | | | |
| | | 55 | | 0.399 | | | 0.249 |
| | 2 | 25 | 0.196 | | | | |
| | | 40 | | | | | |
| | | 55 | | 0.285 | | | 0.202 |
| Formulation 12 | 0 | 25 | 0.048 | | | | |
| | | 40 | | | | | |
| | | 55 | | 0.751 | | | 0.43 |
| | 1 | 25 | 0.203 | | | | |
| | | 40 | | | | | |
| | | 55 | | 0.232 | | | 0.175 |
| | 2 | 25 | 0.193 | | | | |
| | | 40 | | | | | |
| | | 55 | | 0.216 | | | 0.111 |
| Formulation 13 | 0 | 25 | 0.236 | | | | |
| | | 40 | | | | | |
| | | 55 | | 0.94 | | | 0.603 |
| | 1 | 25 | 0.234 | | | | |
| | | 40 | | | | | |
| | | 55 | | 0.207 | | | 0.165 |
| | 2 | 25 | 0.13 | | | | |
| | | 40 | | | | | |
| | | 55 | | 0.121 | | | 0.178 |
| Formulation 14 | — | 25 | 0.136 | | | | |
| | | 40 | | | | | |
| | | 55 | | | | | |
| Formulation 15 | — | 25 | 0.226 | | | | |
| | | 40 | | | | | |
| | | 55 | | | | | |

### III. Example 3: Preparation of Embodiments of the Composition

Unless otherwise noted, the phosphatidylcholine composition over 20% phosphatidylcholine (PC20) or over 90% phosphatidylcholine (PC90).

Particle size and zeta potential of liquid was measured on a Malvern ZS90 Zetasizer (Malvern, UK). Products were measured in low-volume, disposable cuvettes and zeta cassettes. Active agent concentrations, related substances and identity (retention time) were measure by high-pressure liquid chromatography (HPLC) at 374 Labs (Reno, NV). Residual solvents were measured by gas chromatography (GC), and heavy metals by inductive coupled plasma-optical emission spectrometry (ICP-OES) at 374 Labs. Rapid preservative effectiveness testing can be determined by a reduction in colony forming units (CFU) of test microorganisms at Microchem Laboratory (Round Rock, Texas). Preservative effectiveness testing can confirmed that the compositions were resistant to bacterial growth (by measuring colony forming units (CFUs) per volume in a given amount of time.

Manufacturing Process: Formulations containing active agents and varying amounts of plant extract were prepared using a solvent-free manufacturing process, as shown in Table 1. To prepare the composition, water was heated to 75° C. before adding additional ingredients. Sodium bicarbonate was added to adjust the pH. The required amounts of plant extract were dispersed in the aqueous solution at varying concentrations as well as other hydrophilic components. Afterwards, the lipophilic components were added with high shear mixing. The active ingredient was then added to achieve a final concentration of 15 mg/mL. Once high shear mixing was completed, the mixture was high pressure homogenized until a homogeneous mixture was produced.

All water-soluble formulation ingredients were dissolved into deionized water at the specified concentrations. Aqueous solutions were heated and filtered prior to further use. An appropriate amount of aqueous solution was transferred to the glass vessel containing the dried lipid ingredients. The glass vessel was transferred to a heating mantel and warmed with constant stirring from an overhead mixer. Mixing was continued until a homogenous slurry of lipids in water was formed. The full volume of lipid slurry was processed through a microfluidizer (Microfluidics Corporation) 0 to 10 times at a processing pressure of 10,000-30,000 PSI. Alternatively, the volume of lipid slurry can be processed at a pressure of 10,000-30,000 PSI such that the material is recirculated back into the unprocessed volume for a period of time until the desired particle size characteristics are achieved. The resulting lipid nanoparticle solution was cooled with continuous stirring for 12-24 hours before characterizing and fill-finish.

### Iv. Example 4: Preparation of Oil-Free Nanoparticles and Stability Study

Multiple nanoparticles were prepared using the manufacturing process described in Example 3. 250 gram batches containing the concentrations of components described in Table 6 below were prepared and 30 grams were filled into 30 mL dropper cap bottles for stability testing. Samples were stored at controlled 2-8° C., room temperature (25° C./60% relative humidity), or accelerated conditions (40° C./75% relative humidity or 55° C.) for testing. Shown in Table 7 are changes in concentration of the active agent, changes in Z-average particle size, changes in D90 particle size, changes in Polydispersity Index (PDI), and changes in turbidity in beer over the period of time tested for each storage condition. Forced hazing tests of the formulation were conducted by adding a standardized amount of encapsulated active ingredient to a beaker. To the beaker, a standardized amount of room temperature liquid (e.g, beer) was added to the beaker. Stirring by magnetic stir bar was initiated at 500 RPM and maintained for 2 minutes. Afterwards, material was transferred to a turbidity sample cell. The turbidity cell was inserted into the Hach turbidimeter and run for turbidity. The machine was run according to the standard operating procedure. Results are reported in NTU. After a TO turbidity measurement was taken, the sample was stored in the turbidity sample cell at 40° C./75% RH for 5 days. Turbidity readings were taken daily (not including weekends or holidays) during this time. Studies were performed such that neither the first nor fifth day of the study fell on a weekend.

Storage at 40° C./75% is generally understood to increase degradation kinetics by a factor of 4 compared to controlled room temperature based on the Arrhenius equation.

It was found that increases in the concentration of the phosphatidylcholine or plant extract lowers the viscosity of the formulations. In addition, it was found that the presence of the plant extract has the effect of reducing the turbidity of the beer containing the formulation. Also, formulations that are higher in phosphatidylcholine (more PC90 than PC20) or had less total lipid also showed lower turbidity in beer.

TABLE 6

| | Percent of ingredients | | | | | | | | | |
|---|---|---|---|---|---|---|---|---|---|---|
| | Saponin source | | | | 5% | | | | | |
| | and | Potassium | Sodium | Xantho- | Sodium | Malto- | | | | |
| Formulation | concentration | Sorbate | Benzoate | humol | Bicarbonate | dextrin | Ethanol | PC20 | PC90 | Water |
| Formulation 1 - | Camellia sinesis | 0.1 | 0.1 | 1.6 | 0.3 | 6 | 0.4 | 6.6 | 15.4 | 59.296 |
| Xanthohumol | extract, 10% | | | | | | | | | |
| Formulation 2 - | Camellia sinesis | 0.1 | 0.1 | 1.6 | 0.3 | 6 | 0.4 | 2 | 18 | 59.296 |
| Xanthohumol | extract, 10% | | | | | | | | | |
| Formulation 3 - | Camellia sinesis | 0.1 | 0.1 | 1.6 | 0.3 | 6 | 0.4 | 4 | 16 | 59.296 |
| Xanthohumol | extract, 5% | | | | | | | | | |
| Formulation 4 - | Camellia sinesis | 0.1 | 0.1 | 1.6 | 0.3 | 6 | 0.4 | 7.2 | 16.8 | 59.296 |
| Xanthohumol | extract, 7.5% | | | | | | | | | |
| Formulation 5 - | Camellia sinesis | 0.1 | 0.1 | 1.6 | 0.3 | 6 | 0.4 | 4.4 | 17.6 | 59.296 |
| Xanthohumol | extract, 7.5% | | | | | | | | | |
| Formulation 6 - | Camellia sinesis | 0.1 | 0.1 | 1.6 | 0.3 | 6 | 0.4 | 4.8 | 19.2 | 59.296 |
| Xanthohumol | extract, 10% | | | | | | | | | |
| Formulation 7 - | Camellia sinesis | 0.1 | 0.1 | 1.6 | 0.3 | 6 | 0.4 | 2.2 | 19.8 | 59.296 |
| Xanthohumol | extract, 5% | | | | | | | | | |
| Formulation 8 - | Camellia sinesis | 0.1 | 0.1 | 1.6 | 0.3 | 6 | 0.4 | 7.2 | 16.8 | 59.296 |
| Xanthohumol | extract, 5% | | | | | | | | | |
| Formulation 9 - | Camellia sinesis | 0.1 | 0.1 | 1.6 | 0.3 | 6 | 0.4 | 2.4 | 21.6 | 59.296 |
| Xanthohumol | extract, 10% | | | | | | | | | |
| Formulation 10 - | Camellia sinesis | 0.1 | 0.1 | 1.6 | 0.3 | 6 | 0.4 | 6 | 14 | 59.296 |
| Xanthohumol | extract, 5% | | | | | | | | | |
| Formulation 11 - | Camellia sinesis | 0.1 | 0.1 | 1.6 | 0.3 | 6 | 0.4 | 2.4 | 21.6 | 59.296 |
| Xanthohumol | extract, 5% | | | | | | | | | |
| Formulation 12 - | Camellia sinesis | 0.1 | 0.1 | 1.6 | 0.3 | 6 | 0.4 | 6 | 14 | 59.296 |
| Xanthohumol | extract, 10% | | | | | | | | | |
| Formulation 13 - | Camellia sinesis | 0.1 | 0.1 | 1.6 | 0.3 | 6 | 0.4 | 2 | 18 | 59.296 |
| Xanthohumol | extract, 7.5% | | | | | | | | | |

| | Percent of ingredients | | | | | | | | | |
|---|---|---|---|---|---|---|---|---|---|---|
| | Saponin source | | | | 5% Sodium | | | | | |
| | and | Potassium | Sodium | | Bicarbonate | Malto- | | | | |
| Formulation | concentration | Sorbate | Benzoate | Resveratrol | Solution | dextrin | Ethanol | PC20 | PC90 | Water |
| Formulation 17 - | Camellia sinesis | 0.1 | 0.1 | 1.5306 | 0.3 | 6 | 0.4 | 6 | 14 | 66.1979 |
| Resveratrol | extract, 1% | | | | | | | | | |

TABLE 7

| Sample and | | | | | | | | | | |
|---|---|---|---|---|---|---|---|---|---|---|
| Viscosity at | Micro- | | Potency (mg/g) | | | | | Particle size (z-avg, nm) | | |
| T0 on Pass | fluidizer | Temp | | 1 | 2 | 3 | 4 | | 1 | 2 |
| 0 (mPa*s) | Passes | (° C.) | Initial | month | mo. | mo. | mo. | Initial | month | mo. |
| Formulation 1 | 0 | 25 | 14.9513 | 15.5328 | | | | 158.133 | 150.4 | |
| | | 40 | | 14.1881 | | | | | 149.4 | |
| | | 55 | | 14.0706 | 12.0518 | 9.9611 | 10.2604 | | 152.5 | 153.1 |
| | 1 | 25 | 9.8699 | 8.0762 | | | | 124.067 | 114 | |
| | | 40 | | 8.173 | | | | | 114.7 | |
| | | 55 | | 7.7707 | 6.8607 | 5.5511 | 5.5314 | | 111.1 | 109.2 |
| | 2 | 25 | 13.2978 | 13.1539 | | | | 135.7 | 115.9 | |
| | | 40 | | 12.5907 | | | | | 112 | |
| | | 55 | | 11.5092 | 11.3072 | 9.1809 | 10.7569 | | 115.1 | 120.5 |
| Formulation 2, | 0 | 25 | 16.1751 | | | | | 190.133 | | |
| 1125.8 | | 40 | | | | | | | | |
| | | 55 | | 13.5048 | 11.2027 | 12.0667 | | | 162.9 | 165.1 |
| | 1 | 25 | 8.9497 | | | | | 117.333 | | |
| | | 40 | | | | | | | | |
| | | 55 | | 11.8279 | 8.3548 | 7.6272 | 7.0015 | | 112.7 | 113.6 |
| | 2 | 25 | 12.6253 | | | | | 112.9 | | |
| | | 40 | | | | | | | | |
| | | 55 | | 8.7232 | 10.2842 | 10.2577 | 8.9606 | | 112.7 | 118.6 |
| Formulation 3, | 0 | 25 | 14.3621 | | | | | 203.5 | | |
| 394.58 | | 40 | | | | | | | | |
| | | 55 | | 8.3321 | 11.1122 | 10.7643 | 9.7774 | | 180.7 | 187.6 |
| | 1 | 25 | 10.2324 | | | | | 118.2 | | |
| | | 40 | | | | | | | | |
| | | 55 | | 12.0579 | 8.5818 | 8.0674 | 7.2077 | | 117 | 112 |
| | 2 | 25 | 12.3641 | | | | | 115.033 | | |
| | | 40 | | | | | | | | |
| | | 55 | | 10.2199 | 10.8789 | 9.9227 | 8.7577 | | 112.7 | 113.7 |
| Formulation 4, | 0 | 25 | 15.9299 | | | | | 256.133 | | |
| 773.48 | | 40 | | | | | | | | |
| | | 55 | | 15.2474 | 13.5531 | 12.5223 | | | 210.9 | 209.5 |
| | 1 | 25 | 10.7601 | | | | | 115 | | |
| | | 40 | | | | | | | | |
| | | 55 | | 9.7496 | 9.0719 | 8.4529 | 7.4696 | | 123.8 | 118.9 |
| | 2 | 25 | 11.4726 | | | | | 103.8 | | |
| | | 40 | | | | | | | | |
| | | 55 | | 10.6847 | 9.8484 | 9.0266 | 8.4636 | | 109.5 | 119.6 |
| Formulation 5, | 0 | 25 | 13.9311 | | | | | 193.733 | | |
| 5029.3 | | 40 | | | | | | | | |
| | | 55 | | 14.6916 | 13.2262 | 12.3752 | | | 171.3 | 173 |
| | 1 | 25 | 10.4552 | | | | | 107.967 | | |
| | | 40 | | | | | | | | |
| | | 55 | | 9.4005 | 8.2858 | 7.7135 | 7.4604 | | 115.8 | 118.9 |
| | 2 | 25 | 14.0347 | | | | | 103.033 | | |
| | | 40 | | | | | | | | |
| | | 55 | | 12.1165 | 11.9013 | 11.0706 | 9.4602 | | 109.5 | 110.9 |
| Formulation 6, | 0 | 25 | 14.2143 | | | | | 196.367 | | |
| 2249.8 | | 40 | | | | | | | | |
| | | 55 | | 13.4091 | 11.7148 | | | | 179.2 | 178 |
| | 1 | 25 | 9.0091 | | | | | 115.1 | | |
| | | 40 | | | | | | | | |
| | | 55 | | 7.8868 | 6.6246 | | 6.7019 | | 119.4 | 111.9 |
| | 2 | 25 | 14.6115 | | | | | 112.9 | | |
| | | 40 | | | | | | | | |
| | | 55 | | 12.9542 | 11.5199 | | 10.4986 | | 105.1 | 116 |
| Formulation 7, | 0 | 25 | 11.6115 | | | | | 209.633 | | |
| 1615.3 | | 40 | | | | | | | | |
| | | 55 | | 13.8058 | 11.0298 | | 10.4001 | | 194.1 | 193.6 |
| | 1 | 25 | 11.7264 | | | | | 117.967 | | |
| | | 40 | | | | | | | | |
| | | 55 | | 9.0657 | 8.3139 | | 7.9545 | | 115.6 | 111.9 |
| | 2 | 25 | 13.7089 | | | | | 104.7 | | |
| | | 40 | | | | | | | | |
| | | 55 | | 12.0819 | 10.4086 | | 9.6196 | | 105.1 | 107.2 |
| Formulation 8, | 0 | 25 | 17.0468 | | | | | 246.7 | | |
| 2838.8 | | 40 | | | | | | | | |
| | | 55 | | 15.1395 | 12.6902 | | | | 212.1 | 119.1 |
| | 1 | 25 | 10.8573 | | | | | 125.3 | | |
| | | 40 | | | | | | | | |
| | | 55 | | 9.3005 | 7.9223 | | 7.7261 | | 128.7 | 129.2 |
| | 2 | 25 | 12.8712 | | | | | 132.267 | | |
| | | 40 | | | | | | | | |
| | | 55 | | 11.332 | 9.4756 | | 9.5777 | | 135 | 134.2 |
| Formulation 9, | 0 | 25 | 12.6155 | | | | | 198.4 | | |
| 23555 | | 40 | | | | | | | | |
| | | 55 | | 13.5818 | 13.1427 | | | | 185.7 | 190.1 |
| | 1 | 25 | 14.7598 | | | | | 113.1 | | |
| | | 40 | | | | | | | | |
| | | 55 | | 12.1346 | 11.3718 | | 9.3543 | | 127.3 | 125.8 |
| | 2 | 25 | 13.5394 | | | | | 114.9 | | |
| | | 40 | | | | | | | | |
| | | 55 | | 12.957 | 12.2283 | | 10.4518 | | 118.6 | 124.2 |
| Formulation | 0 | 25 | 14.6224 | | | | | 224.5 | | |
| 10, 12080 | | 40 | | | | | | | | |
| | | 55 | | 14.3891 | 13.6398 | | | | 211.4 | 206.6 |
| | 1 | 25 | 10.6838 | | | | | 123.567 | | |
| | | 40 | | | | | | | | |
| | | 55 | | 10.7725 | 10.2847 | | 8.8976 | | 117.3 | 116 |
| | 2 | 25 | 13.5986 | | | | | 120.467 | | |
| | | 40 | | | | | | | | |
| | | 55 | | 12.3076 | 11.4808 | | 9.9201 | | 122.5 | 132.8 |
| Formulation | 0 | 25 | 15.1238 | | | | | 201.6 | | |
| 11, 761.78 | | 40 | | | | | | | | |
| | | 55 | | 14.2411 | 12.8797 | | | | 185.6 | 190.3 |
| | 1 | 25 | 12.3965 | | | | | 116.367 | | |
| | | 40 | | | | | | | | |
| | | 55 | | 11.7657 | 10.3413 | | 8.9108 | | 202.2 | 135.6 |
| | 2 | 25 | 13.5104 | | | | | 105.733 | | |
| | | 40 | | | | | | | | |
| | | 55 | | 11.4105 | 11.1848 | | 9.3993 | | 123.3 | 125.8 |
| Formulation | 0 | 25 | 14.4789 | | | | | 281.067 | | |
| 12, 2642 | | 40 | | | | | | | | |
| | | 55 | | 13.5076 | 14.1542 | | | | 251 | 227 |
| | 1 | 25 | 14.5777 | | | | | 203.533 | | |
| | | 40 | | | | | | | | |
| | | 55 | | 12.5766 | 13.4615 | | | | 203.3 | 205.9 |
| | 2 | 25 | 14.0939 | | | | | 137.9 | | |
| | | 40 | | | | | | | | |
| | | 55 | | 13.2873 | 12.8423 | | | | 161.2 | 169 |
| Formulation | 0 | 25 | 14.6595 | | | | | 175.833 | | |
| 13, 2206.5 | | 40 | | | | | | | | |
| | | 55 | | 11.9255 | 12.5822 | | 10.0414 | | 212.6 | 210.7 |
| | 1 | 25 | 13.3871 | | | | | 134.067 | | |
| | | 40 | | | | | | | | |
| | | 55 | | 8.647 | 8.6124 | | 7.2388 | | 121.7 | 122.6 |
| | 2 | 25 | 13.5766 | | | | | 136.567 | | |
| | | 40 | | | | | | | | |
| | | 55 | | 10.9484 | 11.0178 | | 8.8897 | | 138.6 | 127.8 |
| Formulation | 0 | 25 | 14.6595 | | | | | 201.967 | | |
| 14, 52400 | | 40 | | | | | | | | |
| | | 55 | | 12.0142 | 12.2128 | | | | 185.9 | |
| | 1 | 25 | 13.3871 | | | | | 111.2 | | |
| | | 40 | | | | | | | | |
| | | 55 | | 9.7465 | 9.8743 | | 8.5759 | | 116.1 | 114.4 |
| | 2 | 25 | 13.5766 | | | | | 111.2 | | |
| | | 40 | | | | | | | | |
| | | 55 | | 11.1539 | 11.1526 | | 9.4489 | | 112.7 | 111.7 |
| Formulation | 0 | 25 | 15.0426 | | | | | 319.8 | | |
| 15, 1688.75 | | 40 | | | | | | | | |
| | | 55 | | | | | | | | |
| | 1 | 25 | 13.6908 | | | | | 148.7 | | |
| | | 40 | | | | | | | | |
| | | 55 | | | | | | | | |
| | 2 | 25 | 14.4166 | | | | | 140.3 | | |
| | | 40 | | | | | | | | |
| | | 55 | | | | | | | | |
| Formulation | 0 | 25 | 15.7756 | | | | | 295.2 | | |
| 16 | | 40 | | | | | | | | |
| | | 55 | | | | | | | | |
| | 1 | 25 | 13.4726 | | | | | 134.2 | | |
| | | 40 | | | | | | | | |
| | | 55 | | | | | | | | |
| | 2 | 25 | 10.1282 | | | | | 133.7 | | |
| | | 40 | | | | | | | | |
| | | 55 | | | | | | | | |
| Formulation | 0 | 25 | | | | | | 151.3 | | |
| 17 | | 40 | | | | | | | | |
| | | 55 | | | | | | | | |

| Sample and | | | | | | | | | |
|---|---|---|---|---|---|---|---|---|---|
| Viscosity at | Micro- | | Particle size (z-avg, nm) | | Particle size (D90, nm) | | | | |
| T0 on Pass | fluidizer | Temp | 3 | 4 | | 1 | 2 | 3 | 4 |
| 0 (mPa*s) | Passes | (° C.) | mo. | mo. | Initial | month | mo. | mo. | mo. |
| Formulation 1 | 0 | 25 | | | 362.667 | 311 | | | |
| | | 40 | | | | 286 | | | |
| | | 55 | 148.2 | 151 | | 3000 | 312 | 4000 | 411 |
| | 1 | 25 | | | 220.667 | 153 | | | |
| | | 40 | | | | 194 | | | |
| | | 55 | 108.1 | 112.6 | | 175 | 164 | 166 | 176 |
| | 2 | 25 | | | 235.667 | 291 | | | |
| | | 40 | | | | 191 | | | |
| | | 55 | 114.2 | 115.3 | | 192 | 205 | 217 | 189 |
| Formulation 2, | 0 | 25 | | | 7253.33 | | | | |
| 1125.8 | | 40 | | | | | | | |
| | | 55 | 184.2 | | | 359 | 358 | 4280 | |
| | 1 | 25 | | | 133.333 | | | | |
| | | 40 | | | | | | | |
| | | 55 | 111.1 | 113 | | 187 | 187 | 181 | 188 |
| | 2 | 25 | | | 261 | | | | |
| | | 40 | | | | | | | |
| | | 55 | 112.7 | 119.1 | | 197 | 199 | 206 | 231 |
| Formulation 3, | 0 | 25 | | | 7966.67 | | | | |
| 394.58 | | 40 | | | | | | | |
| | | 55 | 175.2 | 173.2 | | 1200 | 680 | 832 | 583 |
| | 1 | 25 | | | 164 | | | | |
| | | 40 | | | | | | | |
| | | 55 | 109.4 | 112.1 | | 191 | 180 | 189 | 189 |
| | 2 | 25 | | | 121.1 | | | | |
| | | 40 | | | | | | | |
| | | 55 | 112.6 | 115.6 | | 270 | 260 | 235 | 211 |
| Formulation 4, | 0 | 25 | | | 9013.33 | | | | |
| 773.48 | | 40 | | | | | | | |
| | | 55 | 206.9 | | | 535 | 427 | 556 | |
| | 1 | 25 | | | 127.333 | | | | |
| | | 40 | | | | | | | |
| | | 55 | 125.3 | 126.4 | | 212 | 222 | 216 | 221 |
| | 2 | 25 | | | 118.267 | | | | |
| | | 40 | | | | | | | |
| | | 55 | 122.5 | 112.1 | | 203 | 207 | 207 | 203 |
| Formulation 5, | 0 | 25 | | | 7610 | | | | |
| 5029.3 | | 40 | | | | | | | |
| | | 55 | 173 | | | 502 | 4530 | 381 | |
| | 1 | 25 | | | 104.9 | | | | |
| | | 40 | | | | | | | |
| | | 55 | 118.7 | 119.1 | | 196 | 197 | 189 | 211 |
| | 2 | 25 | | | 98.067 | | | | |
| | | 40 | | | | | | | |
| | | 55 | 114.6 | 112.3 | | 182 | 195 | 183 | 185 |
| Formulation 6, | 0 | 25 | | | 7523.33 | | | | |
| 2249.8 | | 40 | | | | | | | |
| | | 55 | | | | 378 | 397 | | |
| | 1 | 25 | | | 147.667 | | | | |
| | | 40 | | | | | | | |
| | | 55 | | 119.3 | | 200 | 199 | | 212 |
| | 2 | 25 | | | 116.8 | | | | |
| | | 40 | | | | | | | |
| | | 55 | | 114.7 | | 185 | 200 | | 338 |
| Formulation 7, | 0 | 25 | | | 4073.67 | | | | |
| 1615.3 | | 40 | | | | | | | |
| | | 55 | | 186.9 | | 5270 | 732 | | 840 |
| | 1 | 25 | | | 149.667 | | | | |
| | | 40 | | | | | | | |
| | | 55 | | 112.3 | | 191 | 177 | | 200 |
| | 2 | 25 | | | 103.6 | | | | |
| | | 40 | | | | | | | |
| | | 55 | | 114.5 | | 183 | 180 | | 339 |
| Formulation 8, | 0 | 25 | | | 8320 | | | | |
| 2838.8 | | 40 | | | | | | | |
| | | 55 | | | | 6920 | 809 | | |
| | 1 | 25 | | | 198.333 | | | | |
| | | 40 | | | | | | | |
| | | 55 | | 129.8 | | 217 | 315 | | 359 |
| | 2 | 25 | | | 259.667 | | | | |
| | | 40 | | | | | | | |
| | | 55 | | 138 | | 260 | 281 | | 316 |
| Formulation 9, | 0 | 25 | | | 463.667 | | | | |
| 23555 | | 40 | | | | | | | |
| | | 55 | | | | 341 | 402 | | |
| | 1 | 25 | | | 143.667 | | | | |
| | | 40 | | | | | | | |
| | | 55 | | 125.6 | | 215 | 244 | | 240 |
| | 2 | 25 | | | 902.667 | | | | |
| | | 40 | | | | | | | |
| | | 55 | | 123.7 | | 184 | 247 | | 230 |
| Formulation | 0 | 25 | | | 8576.67 | | | | |
| 10, 12080 | | 40 | | | | | | | |
| | | 55 | | | | 7010 | 606 | | |
| | 1 | 25 | | | 151 | | | | |
| | | 40 | | | | | | | |
| | | 55 | | 115.4 | | 261 | 269 | | 252 |
| | 2 | 25 | | | 122 | | | | |
| | | 40 | | | | | | | |
| | | 55 | | 140.5 | | 386 | 365 | | 513 |
| Formulation | 0 | 25 | | | 8036.67 | | | | |
| 11, 761.78 | | 40 | | | | | | | |
| | | 55 | | | | 388 | 455 | | |
| | 1 | 25 | | | 144 | | | | |
| | | 40 | | | | | | | |
| | | 55 | | 126.8 | | 9190 | 219 | | 213 |
| | 2 | 25 | | | 134 | | | | |
| | | 40 | | | | | | | |
| | | 55 | | 125.4 | | 214 | 218 | | 230 |
| Formulation | 0 | 25 | | | 9130 | | | | |
| 12, 2642 | | 40 | | | | | | | |
| | | 55 | | | | 8490 | 450 | | |
| | 1 | 25 | | | 566.333 | | | | |
| | | 40 | | | | | | | |
| | | 55 | | | | 555 | 6410 | | |
| | 2 | 25 | | | 211.667 | | | | |
| | | 40 | | | | | | | |
| | | 55 | | | | 506 | 6640 | | |
| Formulation | 0 | 25 | | | 5463.33 | | | | |
| 13, 2206.5 | | 40 | | | | | | | |
| | | 55 | | | | 6010 | 678 | | |
| | 1 | 25 | | | 2141 | | | | |
| | | 40 | | | | | | | |
| | | 55 | | | | 214 | 252 | | |
| | 2 | 25 | | | 278 | | | | |
| | | 40 | | | | | | | |
| | | 55 | | | | 359 | 431 | | |
| Formulation | 0 | 25 | | | 8430 | | | | |
| 14, 52400 | | 40 | | | | | | | |
| | | 55 | | | | 378 | | | |
| | 1 | 25 | | | 107.567 | | | | |
| | | 40 | | | | | | | |
| | | 55 | | | | 190 | 192 | | |
| | 2 | 25 | | | 95.3 | | | | |
| | | 40 | | | | | | | |
| | | 55 | | | | 161 | 180 | | |
| Formulation | 0 | 25 | | | 8900 | | | | |
| 15, 1688.75 | | 40 | | | | | | | |
| | | 55 | | | | | | | |
| | 1 | 25 | | | 312 | | | | |
| | | 40 | | | | | | | |
| | | 55 | | | | | | | |
| | 2 | 25 | | | 244 | | | | |
| | | 40 | | | | | | | |
| | | 55 | | | | | | | |
| Formulation | 0 | 25 | | | 8790 | | | | |
| 16 | | 40 | | | | | | | |
| | | 55 | | | | | | | |
| | 1 | 25 | | | 241 | | | | |
| | | 40 | | | | | | | |
| | | 55 | | | | | | | |
| | 2 | 25 | | | 223 | | | | |
| | | 40 | | | | | | | |
| | | 55 | | | | | | | |
| Formulation | 0 | 25 | | | 333 | | | | |
| 17 | | 40 | | | | | | | |
| | | 55 | | | | | | | |

| | Micro- | | | | | | | | |
|---|---|---|---|---|---|---|---|---|---|
| | fluidizer | | PDI | | | | | Turbidity in Beer | |
| Sample | Passes | Temp (° C.) | Initial | 1 month | 2 mo. | 3 mo. | 4 mo. | Initial | Day 5 |
| Formulation 1 | 0 | 25 | 0.196 | 0.165 | | | | | |
| | | 40 | | 0.161 | | | | | |
| | | 55 | | 0.18 | 0.162 | 0.213 | 0.2 | | |
| | 1 | 25 | 0.27 | 0.207 | | | | | |
| | | 40 | | 0.171 | | | | | |
| | | 55 | | 0.149 | 0.147 | 0.152 | 0.137 | | |
| | 2 | 25 | 0.288 | 0.252 | | | | | |
| | | 40 | | 0.202 | | | | | |
| | | 55 | | 0.149 | 0.153 | 0.178 | 0.158 | 50.8 | 37.7 |
| Formulation 2 | 0 | 25 | 0.249 | | | | | | |
| | | 40 | | | | | | | |
| | | 55 | | 0.184 | 0.181 | 0.223 | | | |
| | 1 | 25 | 0.352 | | | | | | |
| | | 40 | | | | | | | |
| | | 55 | | 0.18 | 0.163 | 0.178 | 0.18 | | |
| | 2 | 25 | 0.307 | | | | | | |
| | | 40 | | | | | | | |
| | | 55 | | 0.195 | 0.181 | 0.236 | 0.188 | 35.2 | 53.6 |
| Formulation 3 | 0 | 25 | 0.2117 | | | | | | |
| | | 40 | | | | | | | |
| | | 55 | | 0.288 | 0.262 | 0.256 | 0.26 | | |
| | 1 | 25 | 0.2857 | | | | | | |
| | | 40 | | | | | | | |
| | | 55 | | 0.148 | 0.167 | 0.193 | 0.177 | | |
| | 2 | 25 | 0.3203 | | | | | | |
| | | 40 | | | | | | | |
| | | 55 | | 0.221 | 0.216 | 0.236 | 0.226 | 20.1 | 33.9 |
| Formulation 4 | 0 | 25 | 0.279 | | | | | | |
| | | 40 | | | | | | | |
| | | 55 | | 0.235 | 0.172 | 0.227 | | | |
| | 1 | 25 | 0.2963 | | | | | | |
| | | 40 | | | | | | | |
| | | 55 | | 0.143 | 0.162 | 0.141 | 0.151 | | |
| | 2 | 25 | 0.329 | | | | | | |
| | | 40 | | | | | | | |
| | | 55 | | 0.157 | 0.153 | 0.143 | 0.157 | 25.5 | 44.1 |
| Formulation 5 | 0 | 25 | 0.1893 | | | | | | |
| | | 40 | | | | | | | |
| | | 55 | | 0.208 | 0.209 | 0.167 | | | |
| | 1 | 25 | 0.2837 | | | | | | |
| | | 40 | | | | | | | |
| | | 55 | | 0.172 | 0.143 | 0.14 | 0.154 | | |
| | 2 | 25 | 0.2913 | | | | | | |
| | | 40 | | | | | | | |
| | | 55 | | 0.168 | 0.185 | 0.158 | 0.181 | 47.7 | 76.5 |
| Formulation 6 | 0 | 25 | 0.2543 | | | | | | |
| | | 40 | | | | | | | |
| | | 55 | | 0.176 | 0.19 | | | | |
| | 1 | 25 | 0.3007 | | | | | | |
| | | 40 | | | | | | | |
| | | 55 | | 0.151 | 0.146 | | 0.155 | | |
| | 2 | 25 | 0.307 | | | | | | |
| | | 40 | | | | | | | |
| | | 55 | | 0.185 | 0.194 | | 0.211 | 43.9 | 58.5 |
| Formulation 7 | 0 | 25 | 0.305 | | | | | | |
| | | 40 | | | | | | | |
| | | 55 | | 0.266 | 0.241 | | 0.267 | | |
| | 1 | 25 | 0.33 | | | | | | |
| | | 40 | | | | | | | |
| | | 55 | | 0.164 | 0.153 | | 0.181 | | |
| | 2 | 25 | 0.374 | | | | | | |
| | | 40 | | | | | | | |
| | | 55 | | 0.194 | 0.191 | | 0.235 | 25.4 | 49.4 |
| Formulation 8 | 0 | 25 | 0.2227 | | | | | | |
| | | 40 | | | | | | | |
| | | 55 | | 0.275 | | | | | |
| | 1 | 25 | 0.411 | | | | | | |
| | | 40 | | | | | | | |
| | | 55 | | 0.189 | | | 0.196 | | |
| | 2 | 25 | 0.3277 | | | | | | |
| | | 40 | | | | | | | |
| | | 55 | | 0.196 | | | 0.203 | 41.3 | 41.1 |
| Formulation 9 | 0 | 25 | 0.1423 | | | | | | |
| | | 40 | | | | | | | |
| | | 55 | | 0.104 | | | | | |
| | 1 | 25 | 0.2497 | | | | | | |
| | | 40 | | | | | | | |
| | | 55 | | 0.142 | | | 0.164 | | |
| | 2 | 25 | 0.2783 | | | | | | |
| | | 40 | | | | | | | |
| | | 55 | | 0.139 | | | 0.163 | 52.5 | 66.9 |
| Formulation | 0 | 25 | 0.2857 | | | | | | |
| 10 | | 40 | | | | | | | |
| | | 55 | | 0.219 | | | | | |
| | 1 | 25 | 0.3293 | | | | | | |
| | | 40 | | | | | | | |
| | | 55 | | 0.253 | | | 0.262 | | |
| | 2 | 25 | 0.3617 | | | | | | |
| | | 40 | | | | | | | |
| | | 55 | | 0.278 | | | 0.3 | 25.7 | 30.6 |
| Formulation | 0 | 25 | 0.202 | | | | | | |
| 11 | | 40 | | | | | | | |
| | | 55 | | 0.17 | | | | | |
| | 1 | 25 | 0.276 | | | | | | |
| | | 40 | | | | | | | |
| | | 55 | | 0.29 | | | 0.15 | | |
| | 2 | 25 | 0.287 | | | | | | |
| | | 40 | | | | | | | |
| | | 55 | | 0.146 | | | 0.166 | 34 | 54.9 |
| Formulation | 0 | 25 | 0.21 | | | | | | |
| 12 | | 40 | | | | | | | |
| | | 55 | | 0.112 | 0.205 | | | | |
| | 1 | 25 | 0.2327 | | | | | | |
| | | 40 | | | | | | | |
| | | 55 | | 0.3 | 0.269 | | | | |
| | 2 | 25 | 0.3023 | | | | | | |
| | | 40 | | | | | | | |
| | | 55 | | 0.268 | 0.239 | | | 56.7 | 55.1 |
| Formulation | 0 | 25 | 0.359 | | | | | | |
| 13 | | 40 | | | | | | | |
| | | 55 | | 0.21 | 0.25 | | | | |
| | 1 | 25 | 0.3387 | | | | | | |
| | | 40 | | | | | | | |
| | | 55 | | 0.167 | 0.194 | | | | |
| | 2 | 25 | 0.3177 | | | | | | |
| | | 40 | | | | | | | |
| | | 55 | | 0.255 | 0.26 | | | 74.4 | 73.8 |
| Formulation | 0 | 25 | 0.2683 | | | | | | |
| 14 | | 40 | | | | | | | |
| | | 55 | | 0.153 | | | | | |
| | 1 | 25 | 0.2963 | | | | | | |
| | | 40 | | | | | | | |
| | | 55 | | 0.157 | 0.158 | | | | |
| | 2 | 25 | 0.3053 | | | | | | |
| | | 40 | | | | | | | |
| | | 55 | | 0.166 | 0.181 | | | 19.6 | 29.3 |
| Formulation | 0 | 25 | 0.179 | | | | | | |
| 15 | | 40 | | | | | | | |
| | | 55 | | | | | | | |
| | 1 | 25 | 0.185 | | | | | | |
| | | 40 | | | | | | | |
| | | 55 | | | | | | | |
| | 2 | 25 | 0.141 | | | | | | |
| | | 40 | | | | | | | |
| | | 55 | | | | | | 50.5 | 49.2 |
| Formulation | 0 | 25 | 0.155 | | | | | | |
| 16 | | 40 | | | | | | | |
| | | 55 | | | | | | | |
| | 1 | 25 | 0.176 | | | | | | |
| | | 40 | | | | | | | |
| | | 55 | | | | | | | |
| | 2 | 25 | 0.119 | | | | | | |
| | | 40 | | | | | | | |
| | | 55 | | | | | | 65.5 | 67.9 |
| Formulation | 0 | 25 | 0.375 | | | | | | |
| 17 | | 40 | | | | | | | |
| | | 55 | | | | | | | |

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A nanoparticle composition comprising water at about 40 wt. % to about 93.998 wt. % and at least one nanoparticle, the nanoparticle comprising:
at least one active agent at 0.001 wt. % to 20 wt. %;
at least one phospholipid at 1 wt. % to 25 wt. %;
at least one oil other than the at least one phospholipid at 3 wt. % to 50 wt. %; and
at least one saponin at 0.001 wt. % to 5 wt. %,
wherein the weight percentages are based on the weight of the composition.

2. The nanoparticle composition of claim 1, wherein the active agent comprises one or more pharmaceutical, nutraceutical, cosmetic, pigment, or flavoring.

3. The nanoparticle composition of any one of claims 1 to 2, wherein the active agent comprises a hydrophobic active ingredient.

4. The nanoparticle composition of any one of claims 1 to 3, wherein the active agent comprises coenzyme Q10, a vitamin E, a vitamin A, a beta carotene, squalene, a vitamin K, a docosahexaenoic acid, a curcuminoid, a phytoceramide, vitamin D2, vitamin D3, and/or an ashwagandha extract.

5. The nanoparticle composition of claim 4, wherein the vitamin K comprises a powdered vitamin K or a vitamin K oil.

6. The nanoparticle composition of any one of claims 1 to 5, wherein the active agent comprises a small molecule and/or a biologic.

7. The nanoparticle composition of any one of claims 1 to 6, wherein the nanoparticle composition does not comprise a starch, a self-emulsifying oil, a co-solvent, a polyethylene glycol, and/or a synthetic surfactant.

8. The nanoparticle composition of any one of claims 1 to 7, wherein the phospholipid comprises a lysophospholipid, a phosphatidylcholine, a phosphatidylinositol, a phosphatidylethanolamine, and/or a phosphatidylserine.

9. The nanoparticle composition of any one of claims 1 to 8, wherein a source of the phospholipid is a lecithin.

10. The nanoparticle composition of any one of claims 1 to 9, wherein the phospholipid comprises hydrogenated and/or non-hydrogenated soybean phosphatidylcholine and/or sunflower phosphatidylcholine.

11. The nanoparticle composition of claim 10, wherein less than 10% of the phosphatidylcholine is hydrogenated.

12. The nanoparticle of any one of claims 1 to 11, wherein the oil other than the at least one phospholipid comprises a plant and/or vegetable oil.

13. The nanoparticle of claim 12, wherein the plant and/or vegetable oil comprises olive oil, sunflower oil, avocado oil, and/or Vitamin E.

14. The nanoparticle composition of any one of claims 1 to 13, wherein the at least one saponin is comprised in a plant extract that is derived from Tribulus terrestris, Yucca schidigera, Quillaja saponaria, Camillia sinesis, and/or Glycyrrhiza glabra.

15. The nanoparticle composition of any one of claims 1 to 14, wherein the at least one saponin is comprised in a plant extract that is a de-oiled plant extract.

16. The nanoparticle composition of any one of claims 1 to 15, wherein the at least one nanoparticle does not comprise a synthetic surfactant.

17. The nanoparticle composition of any one of claims 1 to 16, wherein the composition comprises a mixture of nanoparticles selected from at least two of a multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, micelles, and/or combinations thereof.

18. The nanoparticle composition of any one of claims 1 to 17, comprised in a liquid formulation.

19. The nanoparticle composition of any one of claims 1 to 18, further comprising at least one co-emulsifier and/or at least one preservative.

20. The nanoparticle composition of claim 19, wherein the co-emulsifier is a gum.

21. The nanoparticle composition of claim 20, wherein the gum is a xanthan gum and/or a biosaccharide gum.

22. The nanoparticle composition of any one of claims 20 to 21, wherein the composition comprises 0.01 wt. % to 5 wt. % of the gum, wherein the weight percentages are based on the weight of the composition.

23. The nanoparticle composition of any one of claims 1 to 22, wherein the at least one nanoparticle comprises:
sodium benzoate;
potassium sorbate;
sodium ascorbate;
Vitamin D3;
Vitamin E;
olive oil;
an extract of Camellia sinesis and/or Tribulus terrestris comprising a saponin;
phosphatidylcholine; and
water.

24. A nanoparticle composition comprising water at about 40 wt. % to about 93.998 wt. % and at least one nanoparticle, the nanoparticle comprising:
at least one phospholipid at 1 wt. % to 25 wt. %;
at least one oil other than the at least one phospholipid at 3 wt. % to 45 wt. %; and
at least one saponin at 0.001 wt. % to 5 wt. %,
wherein the weight percentages are based on the weight of the composition.

25. The nanoparticle composition of claim 24, wherein the phospholipid comprises a lysophospholipid, a phosphatidylcholine, a phosphatidylinositol, a phosphatidylethanolamine, and/or a phosphatidylserine.

26. The nanoparticle composition of any one of claims 24 to 25, wherein a source of the phospholipid is lecithin.

27. The nanoparticle composition of any one of claims 24 to 26, wherein the phospholipid comprises hydrogenated and/or non-hydrogenated soybean phosphatidylcholine and/or sunflower phosphatidylcholine.

28. The nanoparticle composition of claim 27, wherein less than 10% of the phosphatidylcholine is hydrogenated.

29. The nanoparticle of any one of claims 24 to 28, wherein the oil other than the at least one phospholipid comprises a plant and/or vegetable oil.

30. The nanoparticle of claim 29, wherein the plant and/or vegetable oil comprises olive oil sunflower oil, avocado oil, and/or Vitamin E.

31. The nanoparticle composition of any one of claims 24 to 30, wherein the at least one saponin is comprised in a plant extract that is derived from Tribulus terrestris, Yucca schidigera, Quillaja saponaria, Camillia sinesis, and/or Glycyrrhiza glabra.

32. The nanoparticle composition of any one of claims 24 to 31, wherein the at least one saponin is comprised in a plant extract that is a de-oiled plant extract.

33. The nanoparticle composition of any one of claims 24 to 32, wherein the at least one nanoparticle does not comprise a synthetic surfactant.

34. The nanoparticle composition of any one of claims 24 to 33, wherein the composition comprises a mixture of nanoparticles selected from at least two of a multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, micelles, and/or combinations thereof.

35. The nanoparticle composition of any one of claims 24 to 34, comprised in a liquid formulation.

36. The nanoparticle composition of any one of claims 24 to 35, further comprising at least one co-emulsifier and/or at least one preservative.

37. The nanoparticle composition of claim 36, wherein the co-emulsifier is a gum.

38. The nanoparticle composition of any one of claims 24 to 37, wherein the nanoparticle composition does not comprise a starch, a self-emulsifying oil, a co-solvent, a polyethylene glycol, and/or a synthetic surfactant.

39. The nanoparticle composition of any one of claims 24 to 38, wherein the at least one nanoparticle comprises:
sodium benzoate;
potassium sorbate;
sodium ascorbate;
Vitamin D3;
Vitamin E;
olive oil;
an extract of Camellia sinesis and/or Tribulus terrestris comprising a saponin;
phosphatidylcholine; and
water.

40. A composition comprising a plurality of lipid nanoparticles comprising:
5 to 60 wt. % of at least one phosphatidylcholine;
0 to 40 wt. % of a bulking agent;
0.01 to 20 wt. % of an active agent; and
0.1 to 20 wt. % of at least one saponin.

41. The composition of claim 40, comprising:
5 to 30 wt. % of a phosphatidylcholine;
0 to 20 wt. % of a bulking agent;
0.1 to 10 wt. % of an active agent;
0.5 to 10 wt. % of at least one saponin; and
25.0 to 93.9 wt. % of water.

42. The composition of claim 40 or 41, wherein the composition does not include a bulking agent.

43. The composition of claim 40 or 41, wherein the composition comprises a bulking agent.

44. The composition of claim 43, wherein the composition comprises about 3.0 wt. % to about 9.0 wt. % of the bulking agent.

45. The composition of any one of claims 43 or 44, wherein the bulking agent comprises a carbohydrate and/or polymer.

46. The composition of claim 45, wherein the bulking agent comprises maltodextrin and/or mannitol.

47. The composition of any one of claims 40 to 46, wherein the at least one saponin is comprised in a plant extract derived from Tribulus terrestris, Yucca schidigera, Quillaja saponaria, Camellia sinesis, and/or Glycyrrhiza glabra.

48. The composition of any one of claims 40 to 47, wherein the at least one saponin is comprised in a plant extract that is a de-oiled plant extract.

49. The composition of any one of claims 40 to 48, further comprising 1-Lysophosphatidylcholine (1-LPC), 2-Lysophosphatidylcholine (2-LPC), Phosphatidylethanolamine (PE), N-acylphosphatidylethanolamine (APE), Phosphatidylinositol (PI), and/or Phosphatidic acid (PA).

50. The composition of claim 49, wherein the concentration of PI and/or PE is significantly greater than the concentration of 1-LPC, 2-LPC, APE, and/or PA.

51. The composition of claim 49, wherein the concentration of 1-LPC, 2-LPC, PE, APE, PI, and/or PA are at or below 1/10^{th} the concentration of phosphatidylcholine.

52. The composition of any one of claims 40 to 51, comprising: about 0 wt. % to about 5.0 wt. % of an antioxidant.

53. The composition of any one of claims 40 to 52, comprising: about 0 wt. % to about 0.5 wt. % of an antioxidant.

54. The composition of any one of claims 40 to 53, wherein greater than about 80% of the active agent is comprised in a lipid nanoparticle.

55. The composition of any one of claims 40 to 54, wherein the composition further comprises at least one or more buffers, one or more solvents, and/or one or more preservatives.

56. The composition of claim 55, wherein the one or more buffers comprises sodium bicarbonate and/or sodium carbonate, the one or more solvents comprises ethanol, and the one or more preservatives comprises citric acid monohydrate, potassium sorbate, sodium benzoate, and/or a natural preservative.

57. The composition of claim 56, comprising:
sodium bicarbonate at about 0.0015 wt % to about 0.06 wt. % and/or sodium carbonate at about 0.0015 wt % to about 0.06 wt. %; and
citric acid monohydrate at about 0.003 wt. % to about 0.4 wt. %, potassium sorbate at about 0.003 wt. % to about 0.4 wt. %, sodium benzoate at about 0.003 wt. to about 0.4 wt %, and/or natural preservative at about 0.003 wt. % to about 2.0 wt. %.

58. The composition of any one of claims 56 to 57, wherein the natural preservative comprises a mushroom extract.

59. The composition of claim 58, wherein the mushroom extract is from a stem of a white button mushroom.

60. The composition of any one of claims 40 to 59, wherein the active agent comprises a hydrophilic active agent.

61. The composition of any one of claims 40 to 59, wherein the active agent comprises a hydrophobic active agent.

62. The composition of any one of claims 40 to 61, wherein the active agent has a greater wt. % solubility in either water or ethanol than in medium chain triglycerides.

63. The composition of any one of claims 40 to 62, wherein the active agent comprises a polyphenol.

64. The composition of any one of claims 40 to 63, wherein the active agent comprises a flavonoid.

65. The composition of claim 64, wherein the flavonoid is a prenylated flavonoid.

66. The composition of claim 65, wherein the prenylated flavonoid is xanthohumol.

67. The composition of any one of claims 40 to 66, wherein the active agent comprises xanthohumol and additional hop prenylated flavonoids.

68. The composition of any one of claims 66 to 67, wherein at least about 90% of the xanthohumol does not isomerize to isoxanthohumol when stored at 25° C. with a relative humidity of about 60%, for at least 1 month.

69. The composition of claim 68, wherein at least about 90% of the xanthohumol does not isomerize to isoxanthohumol when stored at 25° C. with a relative humidity of 60%, for at least 4 months.

70. The composition of claim 68, wherein at least about 90% of the xanthohumol does not isomerize to isoxanthohumol when stored at 25° C. with a relative humidity of 60%, for at least 8 months.

71. The composition of any one of claims 40 to 70, wherein the weight ratio of the phosphatidylcholine to the active agent is about 12:1 to about 3:2.

72. The composition of claim 71, wherein the weight ratio of the phosphatidylcholine to the active agent is about 11:1 to about 5:1.

73. The composition of any one of claims 40 to 72, wherein the phosphatidylcholine is from a sunflower.

74. The composition of claim 73, wherein the phosphatidylcholine comprises a compound with a structure of:

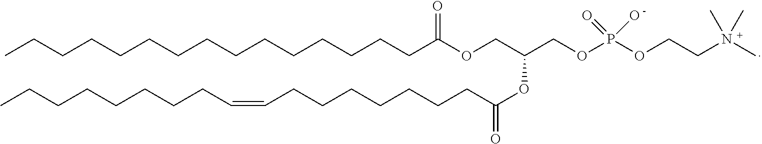

75. The composition of any one of claims 40 to 74, comprising:
5 to 25 wt. % of the phosphatidylcholine;
0.1 to 10 wt. % of the active agent;
0 to 7.5 wt. % of the bulking agent;
2.5 to wt. 10% of at least one saponin; and
25 to 89.9 wt. % water.

76. The composition of any one of claims 40 to 75, comprising:
10 to 25 wt. % the phosphatidylcholine;
1 to about 2.5 wt. % of the active agent;
0 to 5 wt. % the bulking agent;
5 to wt. 10% of at least one saponin; and
50 to about 70 wt. % water.

77. The composition of any one of claims 40 to 76, consisting essentially of the phosphatidylcholine, the active agent, the bulking agent, the at least one saponin, and optionally the water.

78. The composition of any one of claims 75 or 76, consisting of the phosphatidylcholine, the active agent, the bulking agent, the at least one saponin, and the water.

79. The composition of any one of claims 40 to 78, further comprising: about 0 wt. % to about 0.5 wt. % of an antioxidant.

80. The composition of any one of claims 40 to 79, wherein the composition comprises 0.01 to 5.0 wt. % of ethanol.

81. The composition of any one of claims 40 to 80, wherein the composition comprises 0.1 to 1.0 wt. % of ethanol.

82. The composition of any one of claims 40 to 81, wherein the bulking agent comprises a maltodextrin based bulking agent.

83. The composition of any one of claims 40 to 82, wherein the bulking agent comprises a mannitol based bulking agent.

84. The composition of any one of claims 40 to 83, wherein the plurality of lipid nanoparticles has an average density of about 0.993 g/cm³ to about 1.02 g/cm³.

85. The composition of any one of claims 40 to 84, wherein the lipid nanoparticles comprise liposomes and/or emulsion particles.

86. The composition of claim 85, wherein at least about 90% of the plurality of lipid nanoparticles are liposomes.

87. The composition of any one of claims 40 to 86, wherein the plurality of lipid nanoparticles has an average size ranging from about 30 nanometers (nm) to about 200 nm.

88. The composition of any one of claims 40 to 87, wherein the plurality of lipid nanoparticles has an average size ranging from about 50 nm to about 150 nm.

89. The composition of any one of claims 40 to 88, wherein the plurality of lipid nanoparticles has an average size ranging from about 100 nm to about 150 nm.

90. The composition of any one of claims 40 to 89, wherein 25% to 100% of the lipid nanoparticles are liposomes.

91. The composition of any one of claims 40 to 90, wherein 50% to 100% of the lipid nanoparticles are liposomes.

92. The composition of any one of claims 40 to 91, wherein 75% to 100% of the lipid nanoparticles are liposomes.

93. The composition of any one of claims 40 to 92, wherein 95% to 100% of the lipid nanoparticles are liposomes.

94. The composition of any one of claims 40 to 93, wherein the active agent is comprised within either an inner surface of the nanoparticle, within an outer surface of the nanoparticle, and/or within a lipid bilayer of the nanoparticle.

95. The composition of claim 94, wherein the active agent is comprised within the lipid bilayer of the nanoparticle.

96. The composition of any one of claims 40 to 95, wherein the majority of nanoparticles are unilamellar.

97. The composition of any one of claims 40 to 96, comprising multilamellar nanoparticles.

98. The composition of any one of claims 40 to 97, wherein less than about 20% of the lipid nanoparticles are emulsion particles.

99. The composition of any one of claims 40 to 98, wherein less than about 10% of the lipid nanoparticles are emulsion particles.

100. The composition of any one of claims 40 to 99, having a turbidity of about 0 to about 5000 Nephelometric Turbidity Units (NTU) at a temperature of about 4° C. and/or a turbidity of about 0 to about 5000 NTU at a temperature of about 40° C. at a relative humidity of about 75%.

101. The composition of any one of claims 40 to 100, having a turbidity of about 0 to about 2000 NTU at a temperature of about 4° C. and/or a turbidity of about 0 to about 2000 NTU at a temperature of about 40° C. at a relative humidity of about 75%.

102. The composition of any one of claims 40 to 101, having a turbidity of about 100 to about 1000 NTU at a temperature of about 4° C. and/or a turbidity of about 100 to about 1000 NTU at a temperature of about 40° C. at a relative humidity of about 75%.

103. The composition of any one of claims 40 to 102, having a turbidity of about 100 to about 500 NTU at a temperature of about 4° C. and/or a turbidity of about 100 to about 800 NTU at a temperature of about 40° C. at a relative humidity of about 75%.

104. The composition of any one of claims 40 to 103, having a turbidity of about 100 to about 500 NTU at a temperature of about 4° C. and/or a turbidity of about 100 to about 800 NTU at a temperature of about 40° C., when stored for about 1 month at a relative humidity of about 75%.

105. The composition of any one of claims 40 to 104, wherein the composition does not include a sterol and/or a triglyceride.

106. The composition of any one of claims 40 to 105, wherein the composition is free of synthetic surfactants and is free of synthetic emulsifiers at concentrations greater than about 1/10^{th} that of phosphatidylcholine.

107. The composition of claim 106, wherein the composition comprises less than about 2.5 wt. % of emulsifiers other than phosphatidylcholine.

108. The composition of any one of claims 40 to 107, wherein the composition is configured such that, upon storage for a period of one month at room temperature, the average size of the nanoparticles changes by less than about 20%.

109. The composition of any one of claims 40 to 108, wherein polydispersity index (PDI) of the plurality of lipid nanoparticles in the composition is about 0.01 to about 0.8.

110. The composition of any one of claims 40 to 109, wherein PDI of the plurality of lipid nanoparticles in the composition is about 0.05 to about 0.5.

111. The composition of any one of claims 79 to 109, wherein PDI of the plurality of lipid nanoparticles in the composition is about 0.1 to about 0.5.

112. A method of manufacturing a nanoparticle composition of any one of claims 1 to 39, the method comprising the steps of:
(a) adding together one or more phospholipid, one or more oil other than the at least one phospholipid, one or more saponin, optionally one or more active agents, and optionally water;
(b) mixing the ingredients of step (a) creating a mixture;
(c) homogenizing the mixture creating a homogenized mixture;
(d) optionally performing microfluidization on the homogenized mixture creating a microfluid;
(e) optionally sonicating the microfluid creating a sonicated microfluid;
(f) optionally stirring the sonicated microfluid creating a stirred microfluid;
(g) optionally creating a coacervation from the stirred microfluid; and
(h) optionally precipitating the coacervation.

113. The method of claim 112, wherein the mixing of step (b) comprises high sheer mixing.

114. The method of any one of claims 112 to 113, wherein the homogenizing of step (c) comprises high pressure homogenization.

115. The method of any one of claims 112 to 114, wherein the stirring of step (f) comprises mechanical stirring.

116. The method of any one of claims 112 to 115, wherein the precipitating of step (h) comprises solvent precipitation.

117. The method of any one of claims 112 to 116, further comprising drying the nanoparticle composition.

118. The method of claim 117, wherein the drying comprises lyophilizing, spray drying, fluid bed drying, and/or desiccating the nanoparticle composition.

119. A nanoparticle composition comprising water at about 0 wt. % to about wt. 10% and at least one nanoparticle, the nanoparticle comprising:
optionally at least one active agent at 0.0015 wt. % to 40 wt. %;
at least one phospholipid at 1.5 wt. % to 30 wt. %;
at least one oil other than the at least one phospholipid at 7.5 wt. % to 80 wt. %; and
at least one saponin at 0.0015 wt. % to 10 wt. %.

120. The nanoparticle composition of claim 119, wherein the active agent comprises one or more pharmaceutical, nutraceutical, cosmetic, pigment, or flavoring.

121. The nanoparticle composition of any one of claims 119 to 120, wherein the composition comprises a mixture of nanoparticles selected from at least two of a liposome, a micelle, a nanoemulsion, a multi-lamellar particle, a double liposome particle, and a solid lipid particle.

122. The nanoparticle composition of any one of claims 119 to 121, wherein the at least one nanoparticle comprises:
sodium benzoate;
potassium sorbate;
sodium ascorbate;
Vitamin D3;
Vitamin E;
olive oil;
an extract of Camellia sinesis and/or Tribulus terrestris comprising a saponin; and
phosphatidylcholine.

123. The nanoparticle composition of any one of claims 119 to 122, wherein the nanoparticle composition does not comprise a starch, a self-emulsifying oil, a co-solvent, a polyethylene glycol, and/or a synthetic surfactant.

124. An aqueous composition comprising the composition of any one of claims 40 to 162.

125. The aqueous composition of claim 124, wherein the composition is comprised in a second composition, and does not settle or separate from the second composition when stored for at least one month at a temperature of about 4° C. to about 20° C.

126. The aqueous composition of any one of claims 124 to 125, comprising 20 wt. % to about 99.0 wt. % of the lipid nanoparticles.

127. The aqueous composition of any one of claims 124 to 126, comprising 50 wt. % to about 95.0 wt. % of the lipid nanoparticles.

128. The aqueous composition of any one of claims 124 to 127, comprising 60.0 wt. % to about 80.0 wt. % of the lipid nanoparticles.

129. The aqueous composition of any one of claims 124 to 125, comprising 8.0 wt. % to about 30.0 wt. % of the lipid nanoparticles.

130. The aqueous composition of claim 129, comprising 17.0 wt. % to about 21.0 wt. % of the lipid nanoparticles.

131. The aqueous composition of any one of claims 124 to 130, having a turbidity of about 0 to about 5000 NTU at a temperature of about 4° C. and/or a turbidity of about 0 to about 5000 NTU at a temperature of about 40° C. at a relative humidity of about 75%.

132. The aqueous composition of any one of claims 124 to 131, having a turbidity of about 0 to about 2000 NTU at a temperature of about 4° C. and/or a turbidity of about 0 to about 2000 NTU at a temperature of about 40° C. at a relative humidity of about 75%.

133. The aqueous composition of any one of claims 124 to 132, having a turbidity of about 100 to about 1000 NTU at a temperature of about 4° C. and/or a turbidity of about 100 to about 1000 NTU at a temperature of about 40° C. at a relative humidity of about 75%.

134. The aqueous composition of any one of claims 124 to 133, wherein the aqueous composition has a turbidity of about 100 to about 500 NTU at a temperature of about 4° C. and/or a turbidity of about 100 to about 800 NTU at a temperature of about 40° C. at a relative humidity of about 75%.

135. The aqueous composition of any one of claims 124 to 134, wherein the aqueous composition has a turbidity of about 100 to about 500 NTU at a temperature of about 4° C. and/or a turbidity of about 100 to about 800 NTU at a temperature of about 40° C., when stored for 1 month at a relative humidity of about 75%.

136. The aqueous composition of any one of claims 124 to 135, wherein the aqueous composition does not settle or separate when stored for at least one month at a temperature of about 20° C.

137. The aqueous composition of any one of claims 124 to 136, wherein the aqueous composition is configured such that, upon storage for a period of one month at room temperature, the average size of the plurality of lipid nanoparticles changes by less than about 20%.

138. The aqueous composition of any one of claims 124 to 137, wherein polydispersity index (PDI) of the plurality of lipid nanoparticles in the aqueous composition is about 0.01 to about 0.8.

139. The aqueous composition of any one of claims 124 to 138, wherein PDI of the plurality of lipid nanoparticles in the aqueous composition is about 0.05 to about 0.5.

140. The aqueous composition of any one of claims 124 to 139, wherein PDI of the plurality of lipid nanoparticles in the aqueous composition is about 0.10 to about 0.5.

141. The aqueous composition of any one of claims 124 to 140, wherein the aqueous composition maintains a turbidity (EBC) of less than about 20 EBC more than a control second composition over 5 days at about 40° C. with about 75% relative humidity, where the control second composition is the same as the aqueous composition except that the control second composition does not comprise the plurality of lipid nanoparticles.

142. The aqueous composition of any one of claims 124 to 141, wherein the aqueous composition maintains a turbidity (EBC) of less than about 15 EBC more than a control second composition over 5 days at about 40° C. with about 75% relative humidity, where the control second composition is the same as the aqueous composition except that the control second composition does not comprise the plurality of lipid nanoparticles.

143. The aqueous composition of any one of claims 124 to 142, wherein the aqueous composition maintains a turbidity (EBC) of less than about 10 EBC more than a control second composition over 5 days at about 40° C. with about 75% relative humidity, where the control second composition is the same as the aqueous composition except that the control second composition does not comprise the plurality of lipid nanoparticles.

144. The aqueous composition of any one of claims 124 to 143, wherein the aqueous composition maintains a turbidity (EBC) of less than about 7 EBC more than a control second composition over 5 days at about 40° C. with about 75% relative humidity, where the control second composition is the same as the aqueous composition except that the control second composition does not comprise the plurality of lipid nanoparticles.

145. The aqueous composition of any one of claims 124 to 144, wherein the aqueous composition maintains a turbidity (EBC) of less than about 4 EBC more than a control second composition over 5 days at about 40° C. with about 75% relative humidity, where the control second composition is the same as the aqueous composition except that the control second composition does not comprise the plurality of lipid nanoparticles.

146. The aqueous composition of any one of claims 124 to 145, wherein the aqueous composition maintains a turbidity of less than about 2.5 EBC more than a control second composition over 5 days at about 40° C. with about 75% relative humidity, where the control second composition is the same as the aqueous composition except that the control second composition does not comprise the plurality of lipid nanoparticles.

147. The aqueous composition of any one of claims 124 to 146, wherein the aqueous composition maintains a turbidity of less than about 1 EBC more than a control second composition over 5 days at about 40° C. with about 75% relative humidity, where the control second composition is the same as the aqueous composition except that the control second composition does not comprise the plurality of lipid nanoparticles.

148. The aqueous composition of any one of claims 124 to 147, wherein the aqueous composition maintains a turbidity (EBC) of less than about 300% that of a control second composition over 5 days at about 40° C. with about 75% relative humidity, where the control second composition is the same as the aqueous composition except that the control second composition does not comprise the plurality of lipid nanoparticles.

149. The aqueous composition of any one of claims 124 to 148, wherein the aqueous composition maintains a turbidity (EBC) of less than about 250% that a control second composition over 5 days at about 40° C. with about 75% relative humidity, where the control second composition is the same as the aqueous composition except that the control second composition does not comprise the plurality of lipid nanoparticles.

150. The aqueous composition of any one of claims 124 to 149, wherein the aqueous composition maintains a turbidity (EBC) of less than about 200% that of a control second composition over 5 days at about 40° C. with about 75% relative humidity, where the control second composition is the same as the aqueous composition except that the control second composition does not comprise the plurality of lipid nanoparticles.

151. The aqueous composition of any one of claims 124 to 150, wherein the aqueous composition maintains a turbidity (EBC) of less than about 150% that of a control second composition over 5 days at about 40° C. with about 75% relative humidity, where the control second composition is the same as the aqueous composition except that the control second composition does not comprise the plurality of lipid nanoparticles.

152. The aqueous composition of any one of claims 124 to 151, wherein the aqueous composition maintains a turbidity (EBC) of less than about 125% that of a control second composition over 5 days at about 40° C. with about 75% relative humidity, where the control second composition is the same as the aqueous composition except that the control second composition does not comprise the plurality of lipid nanoparticles.

153. The aqueous composition of any one of claims 124 to 152, wherein the aqueous composition maintains a turbidity of less than about 115% that of a control second composition over 5 days at about 40° C. with about 75% relative humidity, where the control second composition is the same as the aqueous composition except that the control second composition does not comprise the plurality of lipid nanoparticles.

154. The aqueous composition of any one of claims 124 to 153, wherein the aqueous composition maintains a turbidity of less than about 110% that of a control second composition over 5 days at about 40° C. with about 75% relative humidity, where the control second composition is the same as the aqueous composition except that the control second composition does not comprise the plurality of lipid nanoparticles.

155. The aqueous composition of any one of claims 124 to 154, wherein the aqueous composition maintains a turbidity (EBC) that does not significantly differ from the turbidity (EBC) of a control second composition over 5 days at about 40° C. with about 75% relative humidity, wherein the control second composition is the same as the aqueous composition except that the control second composition does not comprise the plurality of lipid nanoparticles.

156. An ingestible composition comprising the composition of any one of claims 40 to 111.

157. The ingestible composition of claim 156, wherein the ingestible composition is a beverage.

158. The ingestible composition of any one of claims 156 to 157, comprising at least about 50.0 wt. % water.

159. The ingestible composition of any one of claims 156 to 158, wherein the ingestible composition is a fermented beverage.

160. The ingestible composition of any one of claims 156 to 159, wherein the ingestible composition is a carbonated beverage.

161. The ingestible composition of any one of claims 156 to 160, wherein ingestible composition is a beer.

162. The ingestible composition of claim 161, wherein the composition or aqueous composition reduces the formation of acetyl aldehyde or beta-damascenone in the beer.

163. The ingestible composition of any one of claims 156 to 162, wherein the composition does not include alcohol.

164. The ingestible composition of any one of claims 156 to 162, wherein the composition includes alcohol.

165. The ingestible composition of claim 164, wherein the composition comprises about 0.01 wt. % to about 10.0 wt % alcohol.

166. The ingestible composition of claim 165, wherein the composition comprises about 2.0 wt % to about 9.0 wt. % alcohol.

167. The ingestible composition of claim 166, wherein the composition comprises about 3.0 wt. % to about 8.0 wt. % alcohol.

168. The ingestible composition of any one of claims 156 to 167, wherein the composition comprises an aqueous formulation and the plurality of lipid nanoparticles.

169. The ingestible composition of any one of claims 156 to 168, wherein the ingestible composition comprises about 10 to about 20 milligrams of the plurality of lipid nanoparticles per about 1 gram of the ingestible composition.

170. The ingestible composition of any one of claims 156 to 169, wherein the turbidity of the composition is not perceivable to the unaided eye.

171. The ingestible composition of any one of claims 156 to 170, wherein the pH of the composition is about 2.5 to 9.5.

172. The ingestible composition of any one of claims 156 to 171, wherein the pH of the composition is about 5.5 to about 7.5.

173. The ingestible composition of any one of claims 156 to 172, wherein the pH of the composition is about 6.0 to about 7.0.

174. The ingestible composition of any one of claims 156 to 173, wherein the pH of the composition is about 6.3 to about 6.7.

175. A method of making the ingestible composition of any one of claims 156 to 174, the method comprising combining the composition of any one of claims 40 to 111 with an ingestible item.

176. The method of claim 175, wherein the ingestible item is a beverage.

177. The method of claim 176, wherein the beverage is a beer.

178. The method of any one of claims 175 to 177, wherein the ingestible composition comprises about 10 to about 20 milligrams of the plurality of lipid nanoparticles per about 1 gram of the ingestible item.

179. A method of ingesting the ingestible composition of any one of claims 156 to 174 by a subject, the method comprising ingesting the ingestible composition.

180. The method of claim 179, wherein ingesting the ingestible composition prevents or treats cancer in the subject.

181. The method of any one of claims 179 to 180, wherein ingesting the ingestible composition prevents or treats inflammation in the subject.

182. The method of any one of claims 179 to 181, wherein ingesting the ingestible composition reduces low-density lipoprotein levels and/or increases high-density lipoprotein levels in the subject.

183. The method of any one of claims 179 to 182, wherein ingesting the ingestible composition aids in recovery from a SARS-CoV-2 infection in the subject.

184. A method of treating a disease, a disorder, and/or a symptom in an individual, the method comprising administering to the individual a therapeutically effective amount of the composition of any one of claims 1 to 111 or 119 to 174.

185. The method of claim 184, wherein the disease is an autoimmune disease, a cancer, a degenerative disease, a blood disease, an infection, and/or a deficiency disease.

186. The method of any one of claims 184 to 185, wherein the symptom comprises opioid withdrawal, pain, anxiety, depression, insomnia, inflammation, fever, fatigue, muscle aches, or a combination thereof.

187. The method of any one of claims 184 to 186, wherein the administering step comprises local administration.

188. The method of any one of claims 184 to 187, wherein the administering step comprises systemic administration.

189. The method of any one of claims 184 to 188, wherein the administering step comprises oral administration.

190. The method of any one of claims 184 to 189, wherein the therapeutically effective amount of the composition comprises 10 mg/kg of the individual to 200 mg/kg of the individual.

191. A method of distributing an active agent in a solution, the method comprising contacting the solution with the composition of any one of claims 1 to 111 or 119 to 174.

192. The method of claim 191, wherein the nanoparticle composition comprises a mixture of nanoparticles selected from at least two of a liposome, a micelle, a nano emulsion, a multi-lamellar particle, a double liposome particle, and a solid lipid particle.

193. A method of adjusting a density of a nanoparticle composition, the method comprising adjusting the density of the nanoparticle composition of any one of claims 1 to 111 or 119 to 174 by adjusting a ratio of at least two of a liposome, a micelle, a nanoemulsion, a multi-lamellar particle, a double liposome particle, and a solid lipid particle.
